(19) United States
(12) Patent Application Publication
(10) Pub. No.: US 2025/0213575 A1
(43) Pub. Date: Jul. 3, 2025

(54) 8-HETEROCYCLIC-3-(PHENYLSULFONYL)-[1,2,3]TRIAZOLO[1,5A]QUINAZOLIN-5(4H)-ONE DERIVATIVES AS ANTI-BACTERIALS

(71) Applicant: BIOVERSYS AG, Basel (CH)

(72) Inventors: Marilyne BOUROTTE, Perenchies (FR); Glenn E. DALE, Basel (CH); Olivier DEFERT, Lille (FR); Marc GITZINGER, Basel (CH); Sergio LOCIURO, Basel (CH); Alessia MICHELOTTI, La Madeleine (FR); Andrej TRAUNER, Basel (CH); Vincent TREBOSC, Kembs (FR)

(21) Appl. No.: 18/875,383

(22) PCT Filed: Jun. 26, 2023
(86) PCT No.: PCT/EP2023/067241

§ 371 Date: Dec. 16, 2024

(30) Foreign Application Priority Data
Jun. 27, 2022 (EP) 22181344.7

(51) Int. Cl. A61K 31/519; A61K 31/5383; A61K 31/5386; A61K 31/551; C07D 487/04; C07D 519/00

(52) U.S. Cl. CPC A61K 31/519; A61K 31/5383; A61K 31/5386; A61K 31/551; C07D 487/04; C07D 519/00

(57) ABSTRACT

The present invention relates to 8-heterocyclic-3-(phenylsulfonyl)-[1,2,3]triazolo[1,5a]quinazolin-5(4H)-one derivatives and pharmaceutical compositions thereof as well as to their uses in methods of reducing the virulence of bacteria (preferably Staphylococcus aureus) that express accessory gene regulator A (AgrA) or an ortholog of AgrA; in methods of inhibition of the quorum sensing in bacteria, preferably in S. aureus; and in methods for preventing or treating diseases caused or exacerbated by bacteria, preferably by S. aureus, such as skin or lung infections, atopic dermatitis, Netherton syndrome or psoriasis in a subject. The compounds exhibited superior ability to inhibit the transcription of genes under the control of the P3 promoter in S. aureus even in the presence of human serum or human serum albumin.

The present invention relates to 8-heterocyclic-3-(phenylsulfonyl)-[1,2,3]triazolo[1,5a]quinazolin-5(4H)-one derivatives and pharmaceutical compositions thereof as well as to their uses in methods of reducing the virulence of bacteria (preferably Staphylococcus aureus) that express accessory gene regulator A (AgrA) or an ortholog of AgrA; in methods of inhibition of the quorum sensing in bacteria, preferably in S. aureus; and in methods for preventing or treating diseases caused or exacerbated by bacteria, preferably by S. aureus, such as skin or lung infections, atopic dermatitis, Netherton syndrome, or psoriasis in a subject. Thus, the present invention relates to anti-virulence compositions and methods for treatment, amelioration and/or prevention of diseases caused or exacerbated by bacteria, preferably by S. aureus, and more particularly to compositions and methods for reducing the virulence of bacteria that express AgrA or an ortholog of AgrA, preferably AgrA.

### RELATED ART

Staphylococcus aureus is both a human commensal and a notorious opportunistic pathogen causing serious community-acquired and hospital-acquired infections. S. aureus is capable of causing a vast array of infections ranging from mild superficial skin infections to severe systemic life-threatening conditions such as endocarditis, pneumonia or sepsis (Lee A S, et al. (2018) Nat. Rev. Dis. Primers, Vol 4, Article 18033 pp. 1-23. Moreover, S. aureus has also been implicated in contributing to allergic skin conditions such as atopic dermatitis (Geoghegan J A, et al. (2018) Trends Microbiol. 26(6):484-497) or Netherton syndrome (Williams M R, et al. (2020) Cell reports 30 (9): 2923-2933). The success of S. aureus to cause such a variety of diseases is a consequence of the extensive arsenal of virulence factors produced combined with β-lactam resistance and, for most clones, resistance to other antibiotic classes. Clinically relevant antibiotic resistance has evolved against virtually every antibiotic deployed, while the discovery and development of novel antibiotic classes is lagging behind, provoking the antibiotic resistance crisis we are facing today. Consequently, alternative strategies to treat or prevent S. aureus-mediated bacterial infections that are also efficacious against multidrug-resistant strains, such as the methicillin-resistant S. aureus (MRSA), are needed (Dickey S W, et al. (2017) Nat. Rev. Drug Discov. 16(7):457-471).

One of these strategies is the anti-virulence approach by which only virulence-associated, but not survival/fitness-relevant traits are targeted. In contrast to common antibiotic therapies, anti-virulence drugs are not per se bacteriostatic (inhibiting bacterial growth) or bactericidal (killing bacteria). This approach focuses on disarming the pathogenic bacteria by blocking the expression or neutralizing their virulence factors, ultimately interfering with bacterial pathogenicity mechanisms and thereby promoting pathogen clearance by the host immune system. Because anti-virulence drugs do not interfere with essential mechanisms of bacterial growth and survival, they are supposed to alleviate the pressure on the pathogen to develop resistance. A further advantage is that specific anti-virulence drugs preserve the healthy host microbiota, and eventually even help to counteract microbial dysbiosis by tuning down the aggressiveness of pathogens such as S. aureus. Importantly, anti-virulence approaches offer an increased repository of pharmacological targets, and thus the possibility of generating alternative antimicrobials with novel mode of action (Muhlen, S. & Dersch, P. (2016) Curr. Top. Microbiol. Immunol. 398:147-183).

How the virulence factors are regulated in S. aureus: The agr operon is a bacterial quorum-sensing system that controls cell-density dependent virulence factor expression in S. aureus. It consists of two divergent promoters, P₂ and P₃, where P₂ is responsible for producing the components of the quorum-sensing system (AgrB, AgrD, AgrC, and AgrA; see FIG. 1). The precursor peptide AgrD is processed by AgrB to form the mature auto-inducing peptide (AIP) that is secreted across the bacterial membrane. AIP binds to the histidine-kinase AgrC and activates the transcriptional regulator AgrA by means of phosphorylation, driving the expression from P₂ and P₃. There are four allelic variants (types I-IV) of agr, each encoding a distinct AIP, which functions as specific ligand for the AgrC of its own cell, but as an inhibitor of other AgrC variants. P₃ produces the agr effector molecule RNAIII that together with AgrA is responsible for transcriptional control of approximately 200 genes including multiple virulence factors and metabolic pathways involved in stationary phase growth (Khan, B A, et al. (2015) Expert Opin. Investig. Drugs 24(5):689-704). Examples for AgrA-regulated virulence factors are cell-surface associated proteins, such as protein A (SpA) and fibronectin-binding proteins, secreted toxins such as α-hemolysin/α-toxin (Hla), δ-hemolysin (Hld), phenol-soluble-modulins (PSMs), Panton-Valentine leukocidin (PVL), leukotoxin E and D (LukED), leukotoxin G and H (LukGH), or secreted proteases such as SspA or aureolysin. Taken together, the numerous and multi-functional virulence determinants make S. aureus pathogenesis particularly complex and provide the pathogen with an arsenal of mechanisms to damage the host or circumvent and evade the host immune defenses. Important to note, most AgrA-regulated virulence factors are causative for the S. aureus pathogenicity in skin and soft tissue infections (SSTIs), lung infections, and were also shown to contribute to chronic inflammatory skin diseases, such as atopic dermatitis (Oliveira D. et al. (2018) Toxins 10: 252; Geoghegan J A, et al. (2018) Trends Microbiol. 26(6):484-497) and Netherton syndrome (Williams M R, et al. (2020) Cell reports 30 (9): 2923-2933).

How to prevent virulence factor expression: Inhibition of expression of the central regulatory RNAIII combined with inhibition of PSMa production is believed to lead to a potent global reduction in levels of virulence factors (see FIG. 1). Current strategies to suppress RNAIII expression can be grouped into different categories: (1) competitive inhibitors of histidine kinase AgrC; (2) RNAIII transcription inhibitors (precise mechanisms undetermined); and (3) inhibition of AgrA-P₂/P₃ interactions. Targeting AgrA has the advantage of blocking AgrA-dependent virulence factor expression on all four agr groups (Gordon C P, et al. (2013) J. Med. Chem. 56(4):1389-404).

An AgrA inhibitor termed Savirin was found by Sully and colleagues in a screen of 24′087 compounds selected for inhibition of cyclic thiolactone peptide pheromone (AIP)-induced agr in the context of studies related to acute bacterial skin and soft tissue infections caused by S. aureus (Sully E K, et al. (2014) PLoS Pathog. 10(6): e1004174). Savirin was shown to be a potent modulator of AgrA-regulated toxin gene transcription such as hla, psma and pvl across all four agr groups without affecting viability of S. aureus. Savirin inhibited exotoxin-induced red blood cell (RBC) lysis. No resistance was developed after multiple passages with Savirin. The molecule was shown to interfere with the AgrA-DNA interaction, preventing virulence gene upregulation. A significant reduction in abscess size and dermonecrosis was observed in mice infected with a MRSA USA300 type strain when Savirin was applied multiple times.

Recently, WO 2020/109350 described 3-(phenylsulfonyl)-[1,2,3]triazolo[1,5a]quina-zolin-5(4h)-one derivatives that are capable of reducing the virulence of bacteria such as S. aureus by interacting with AgrA and inhibiting the expression of AgrA-regulated virulence factors, and its uses for preventing or treating bacterial infections and/or diseases caused or exacerbated by bacteria, preferably by S. aureus, such as skin or lung infections, atopic dermatitis or psoriasis.

### SUMMARY OF THE INVENTION

The inventors have surprisingly found that the ability to inhibit AgrA-related virulence factors by compounds of WO 2020/109350 could be affected and reduced when tested in the presence of human serum (HS) or human serum albumin (HSA; the major component of human serum). Without wishing to be bound, the reduced ability to inhibit expression of AgrA-related virulence factors is thought to be due to nonspecific binding of the compounds to serum proteins (e.g., HSA). Such reduced efficacy in the presence of HS or HSA can limit the ability to inhibit AgrA-related virulence factors when administered systemically (e.g., in an animal, preferably a mammal, more preferably a human). The present invention now provides a new class of compounds overcoming said drawbacks, and thus provides compounds that can reduce the virulence of bacteria, preferably that express AgrA and/or inhibit quorum sensing in bacteria, preferably in S. aureus, in biologically relevant systems that include human serum or human serum albumin.

Thus, the inventors have surprisingly identified a new class of compounds able to interact with AgrA and inhibit the expression of AgrA-regulated virulence factors even in the presence of human serum (HS) or human serum albumin (HSA). The inventors transfected S. aureus with a plasmid expressing the reporter gene lacZ under the control of the P₃ promoter, which is regulated by AgrA. In the presence of the inventive compounds, the inventors found that production of the gene product of lacZ (i.e., β-galactosidase) was inhibited. Surprisingly, the inventive compounds retained AgrA inhibition activity even when the assay was performed in the presence of HS or HSA.

The inventive compounds can thus inhibit the transcription of genes (preferably virulence factors) under the control of the P₃ promoter (e.g., in bacteria, preferably in S. aureus) in the presence of HS or HSA. This activity strongly suggests that the inventive compounds can be administered systemically (e.g., orally or parenterally) in biologically relevant systems (e.g., in animals, preferably mammals, more preferably humans) while still maintaining efficacy. Additional features and advantages of the present technology will be understood by those of skill in the art upon reading the Detailed Description of the Invention, below.

In a first aspect, the present disclosure provides a compound of Formula (I), or a pharmaceutically acceptable salt, solvate, hydrate, or tautomer thereof:

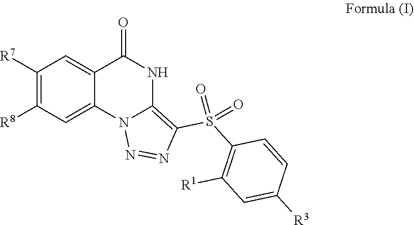

wherein:
R¹ is independently selected from —H, halogen, —C₁-C₆ alkyl, and —C₁-C₆ alkoxy, wherein said alkyl and alkoxy is each optionally substituted with one or more R¹¹;
R³ is independently selected from —H, —C₁-C₆ alkyl, and —C₁-C₆ alkoxy, wherein said alkyl and alkoxy is each optionally substituted by one or more R¹¹;
R⁷ is independently selected from —H and —OH;
R⁸ is a group of the Formula (A):

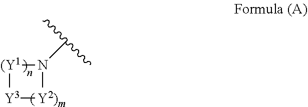

wherein n is independently an integer from 0 to 3;
m is independently an integer from 0 to 3;
Y¹ is independently, at each occurrence, selected from CR⁸¹R⁸², C(O), and O;
Y² is independently, at each occurrence, selected from CR^{81A}R^{82A}, and O;
Y³ is independently selected from CR⁸⁴R⁸⁵; CHR⁸⁶; NR⁸⁷, and O;
wherein the sum of m and n is at least 2 when Y³ is CR⁸⁴R⁸⁵ and wherein the sum of m and n is at least 3 when Y³ is CHR⁸⁶, NR⁸⁷, or O;
R⁸¹, R⁸², R^{81A} and R^{82A} are independently, at each occurrence, selected from —H and —C₁-C₆ alkyl, or one of R⁸¹ and R^{81A}, together with the carbon atoms to which they are attached, can combine to form a bridging ethylene;
R⁸⁴ and R⁸⁵, together with the carbon atom to which they are attached, combine to form a —C₄-C₇ cycloalkyl or a 4- to 7-membered heterocycle, wherein said cycloalkyl and heterocycle is each optionally substituted with one or more halogen, —C₁-C₆ alkyl, —C₁-C₆ haloalkyl, —OH, or —COOH;
R⁸⁶ is independently selected from —H, —COOH, and —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH;
or R⁸⁷, together with the nitrogen atom to which it is attached, can combine with one of R⁸¹, R^{81A}, R⁸² or R^{82A} and the carbon atoms to which they are attached to form a 5- to 6-membered heterocycle or 5- to 6-membered heteroaryl, wherein said heterocycle or heteroaryl is each optionally substituted with one or more halogen, —C₁-C₆ alkyl, —C₁-C₆ haloalkyl, or —OH; and
R¹¹ is independently, at each occurrence, halogen or —OH.

In one aspect, the present disclosure provides a compound of Formula (I), or a pharmaceutically acceptable salt, solvate, hydrate, or tautomer thereof:

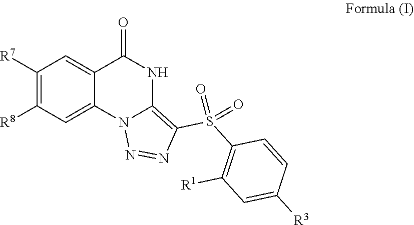

wherein:
R¹ is independently selected from —H, halogen, —C₁-C₆ alkyl, and —C₁-C₆ alkoxy, wherein said alkyl and alkoxy is each optionally substituted with one or more R¹¹;
R³ is independently selected from —H, —C₁-C₆ alkyl, and —C₁-C₆ alkoxy, wherein said alkyl and alkoxy is each optionally substituted by one or more R¹¹;
R⁷ is independently selected from —H and —OH;
R⁸ is a group of the Formula (A):

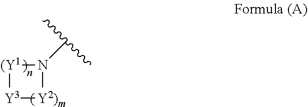

wherein n is independently an integer from 0 to 3;
m is independently an integer from 0 to 3;
Y¹ is independently, at each occurrence, selected from CR⁸¹R⁸², C(O), and O;
Y² is independently, at each occurrence, selected from CR^{81A}R^{82A}, and O;
Y³ is independently selected from CR⁸⁴R⁸⁵; CHR⁸⁶; NR⁸⁷, and O;
wherein the sum of m and n is at least 2 when Y³ is CR⁸⁴R⁸⁵ and wherein the sum of m and n is at least 3 when Y³ is CHR⁸⁶, NR⁸⁷, or O;
R⁸¹, R⁸², R^{81A} and R^{82A} are independently, at each occurrence, selected from —H and —C₁-C₆ alkyl, or one of R⁸¹ and R^{81A}, together with the carbon atoms to which they are attached, can combine to form a bridging ethylene;
R⁸⁴ and R⁸⁵, together with the carbon atom to which they are attached, combine to form a —C₄-C₇ cycloalkyl or a 4- to 7-membered heterocycle, wherein said cycloalkyl and heterocycle is each optionally substituted with one or more halogen, —C₁-C₆ alkyl, —C₁-C₆ haloalkyl, or —OH;
R⁸⁶ is independently selected from —H, and —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH;
or R⁸⁷, together with the nitrogen atom to which it is attached, can combine with one of R⁸¹, R^{81A}, R⁸² or R^{82A} and the carbon atoms to which they are attached to form a 5- to 6-membered heterocycle or 5- to 6-membered heteroaryl, wherein said heterocycle or heteroaryl is each optionally substituted with one or more halogen, —C₁-C₆ alkyl, —C₁-C₆ haloalkyl, or —OH; and
R¹¹ is independently, at each occurrence, halogen or —OH.

In one aspect, the invention provides an inventive compound as described herein or a pharmaceutical composition comprising an inventive compound as described herein for use in a method of reducing the virulence of a bacteria, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In one aspect, the invention provides an inventive compound as described herein or a pharmaceutical composition comprising an inventive compound as described herein for use in a method of inhibiting the quorum sensing in bacteria, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In one aspect, the invention provides an inventive compound as described herein or a pharmaceutical composition comprising an inventive compound as described herein for use in a method of preventing or treating diseases caused or exacerbated by bacteria, preferably a bacteria of a genus selected from Staphylococcus, Streptococcus or Clostridium, more preferably of Staphylococcus, and again further preferably is Staphylococcus aureus, preferably wherein the disease is a skin disease or a lung disease, more preferably wherein the skin disease is atopic dermatitis, Netherton syndrome, or psoriasis.

In one aspect, the present disclosure provides a pharmaceutical composition comprising at least one compound according to Formula (I), or a pharmaceutically acceptable salt, tautomer, solvate or hydrate thereof, and a pharmaceutically acceptable excipient.

In one aspect, the present disclosure provides a compound according to Formula (I) or a pharmaceutically acceptable salt, tautomer, solvate or hydrate thereof, or a pharmaceutical composition comprising a compound according to Formula (I), for use as a medicament.

### DESCRIPTION OF THE FIGURES

FIG. 1 shows quorum sensing signaling in staphylococci to control virulence factor production. The precursor peptide AgrD is processed by AgrB and the mature auto-inducing peptide (AIP) is secreted across the bacterial membrane. AIP binds to the histidine-kinase AgrC of its own cell or on other bacterial cells. AgrC then activates response regulator AgrA by means of phosphorylation. Phospho-AgrA binds to and activates transcription from the agr P₂ and P₃ promoters, as well as the promoters of the psm operons (RNAIII-independent AgrA regulation). The classic targets of AgrA are under RNAIII-dependent control, comprising several toxins and proteases that are up-regulated and several surface-binding proteins such as protein A, that are down-regulated. Small molecule inhibitors that bind to the DNA-binding domain of AgrA prevent the binding to P₂ and P₃ promoter, thus blocking continuous production of AIP (P₂ driven) and virulence factors (P₃ driven) but also block expression of RNAIII-independent AgrA regulation such as production of PSMs.

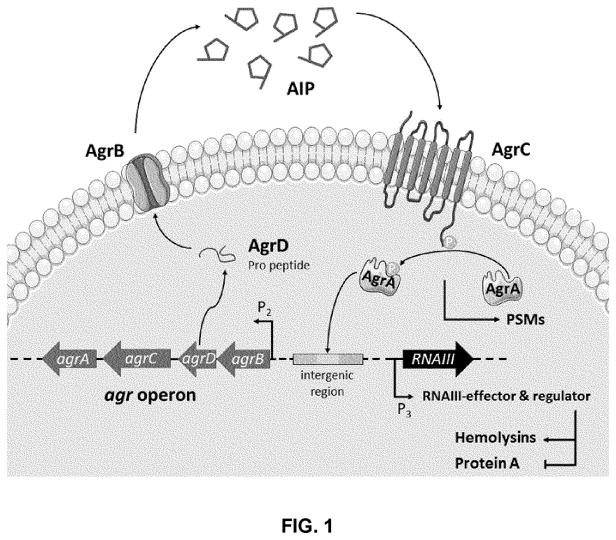

### DETAILED DESCRIPTION OF THE INVENTION

The present invention relates to compounds and pharmaceutical compositions comprising the same that can inhibit the transcription of genes under the control of the P₃ promoter (e.g., in bacteria, preferably in S. aureus) even in the presence of HS or HSA.

### Definitions

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to or its repetition is avoided for the sake of conciseness.

The articles “a” and “an” are used in this disclosure to refer to one or more than one (i.e., to at least one) of the grammatical object of the article unless the context clearly dictates otherwise. By way of example, “an element” means one element or more than one element.

The term “and/or” is used in this disclosure to mean either “and” or “or” unless indicated otherwise.

The term “optionally substituted” is understood to mean that a given chemical moiety (e.g. an alkyl group) can (but is not required to) be bonded other substituents (e.g. heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups.

The term “alkyl” refers to a straight or branched chain saturated hydrocarbon. C₁-C₆ alkyl groups contain 1 to 6 carbon atoms. Examples of a —C₁-C₆ alkyl group include, but are not limited to, methyl, ethyl, propyl, butyl, pentyl, isopropyl, isobutyl, sec-butyl and tert-butyl, isopentyl and neopentyl.

The term “cycloalkyl” means monocyclic saturated carbon ring containing 3-7 carbon atoms. Examples of cycloalkyl groups include, without limitations, cyclopropyl, cyclobutyl, cyclopentyl, cyclohexyl, and cycloheptyl.

Unless otherwise specifically defined, “heteroaryl” means a monovalent monocyclic aromatic radical of 5 to 6 ring atoms containing one or more ring heteroatoms selected from N, S, P, and O, the remaining ring atoms being C. Preferably the heteroatom is selected from N, S, and O, more preferably N and O. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, and pyrazinyl. The heteroaryl group can be fused to a partially saturated heterocycle, e.g., as in Compound 54. Additional exemplary ring systems of such heterocyle-aryl or heterocyle-heteroaryl groups include indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, 2,3,4,5-tetrahydro-1H-pyrido[4,3-b]indole, 5,6,7,8-tetrahydro-imidazo[1,2-a]pyrazine, and dihydrobenzoxanyl.

The terms “heterocyclyl” or “heterocycloalkyl” or “heterocycle” refer to monocyclic or bicyclic saturated or partially saturated 4 to 11-membered rings containing carbon and heteroatoms taken from O, N, and S (preferably O and N) and wherein the ring or rings do not comprise delocalized π electrons (aromaticity) shared among the ring carbons or heteroatoms. Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, piperidinyl, morpholinyl, thiomorpholinyl, piperazinyl, [1,4]diazepane, and [1,2]diazepane. A heterocyclyl or heterocycloalkyl ring can be bridged, e.g., as shown in Compound 50.

As shown in Compounds 3-21, when Y³ is CR⁸⁴R⁸⁵, R⁸ forms a spirocyclic heterocycle with the ring of Formula (A). As used herein a spirocyclic heterocycle or spiroheterocycle is understood to mean a bicyclic ring system in which both rings are connected through a single atom, and wherein at least one of the rings is a heterocycle (e.g., at least one of the rings is azetidine, pyrrolidine, morpholine, or piperidine).

As used herein, the term “halo” or “halogen” means fluoro (F), chloro (Cl), bromo (Br), or iodo (I).

The term “oxo” refers to a carbonyl functional group composing a carbon atom double-bonded to an oxygen atom. It can be abbreviated herein as “oxo”, as C(O), or as C═O.

The invention also includes pharmaceutical compositions comprising an effective amount of a disclosed compound and a pharmaceutically acceptable carrier. The terms “pharmaceutically acceptable” or “therapeutically acceptable” refers to a substance which does not interfere with the effectiveness or the biological activity of the active ingredients and which is not toxic to the host. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, hydroiodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “tautomers” refers to a set of compounds that have the same number and type of atoms, but differ in bond connectivity and are in equilibrium with one another. A “tautomer” is a single member of this set of compounds. Typically, a single tautomer is drawn but it is understood that this single structure is meant to represent all possible tautomers that might exist. Examples include enol-ketone tautomerism. When a ketone is drawn it is understood that both the enol and ketone forms are part of the present disclosure.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as “hydrates.” Hydrates include compositions containing stoichiometric amounts of water, as well as compositions containing variable amounts of water.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “ortholog”, as used herein, denotes the well-known meaning of this term. In this art, orthologs are genes in different species which evolved from a common ancestral gene. Due to their separation following a speciation event, orthologs may diverge, but usually have similarity at the sequence and structure levels; furthermore, orthologs usually have identical functions. Orthology is a type of homology. In this application, the term ortholog is used to include the ortholog gene (DNA or RNA) or the peptide/protein product of the ortholog. Sometimes the peptide/protein product of the ortholog is referred to as “ortholog product’ or simply “ortholog”. The meaning is evident from the context (e.g., an anti-virulence compositions of the present invention may include an anti-virulence agent capable of reducing the virulence of bacterium that expresses peptides or proteins that may be referred to as orthologs of AgrA—that is, products of an ortholog gene of Staphylococcus aureus AgrA from another bacterium, such as Streptococcus pyogenes). In certain aspects, an ortholog of AgrA produces proteins/peptides that share greater than about 70%, about 80%, or about 90% identity with the amino acid sequence of the gene product of AgrA.

The terms “reducing” and “inhibiting” have their commonly understood meaning of lessening or decreasing.

The expression “reducing the virulence of bacteria that express AgrA”, as used herein, typically and preferably, refers to inhibiting the synthesis of one or more virulence factors by said bacteria by the inventive compounds of formula (I) or the inventive compositions, preferably pharmaceutical compositions, comprising an inventive compound of formula (I). Examples for AgrA-regulated virulence factors are cell-surface associated proteins, such as protein A (SpA) and fibronectin-binding proteins, secreted toxins such as α-hemolysin/α-toxin (Hla), δ-hemolysin (Hld), phenol-soluble-modulins (PSMs), Panton-Valentine leukocidin (PVL), leukotoxin E and D (LukED), leukotoxin G and H (LukGH) or secreted proteases such as SspA or aureolysin. In a preferred example and embodiment of the present invention, said reducing the virulence of bacteria that express AgrA is inhibiting the synthesis of one or more virulence factor selected from PSMa, RNAIII and its downstream targets. In a preferred example and embodiment of the present invention, said reducing the virulence of bacteria that express AgrA is inhibiting the synthesis of PSMa. In a preferred example and embodiment of the present invention, said reducing the virulence of bacteria that express AgrA is inhibiting the synthesis of RNAIII and/or its downstream targets, preferably of RNAIII.

The term “inhibiting the synthesis of one or more virulence factors” as used herein shall refer to a complete or partial inhibition (preferably more than 20%, further preferably more than 30%, further preferably more than 50%, further preferably more than 90%, still more preferably more than 95% or even more than 99%) of the synthesis of one or more virulence factors, as compared to the synthesis of one or more virulence factors by said bacteria in the absence of the inventive compounds of formula (I) or the inventive compositions, preferably pharmaceutical compositions, comprising an inventive compound of formula (I) or as compared to an inventive method where no such inventive compounds of formula (I) or the inventive compositions, preferably pharmaceutical compositions, comprising an inventive compound of formula (I) are applied or used.

Virulence factors as contemplated herein include any molecules expressed and secreted by bacteria to promote colonization and/or adhesion in a host subject, promote inflammation in the host tissue, promote evasion of the host's immune response and obtain nutrition from the host subject. Virulence factors can also include both exotoxins and endotoxins. Non-limiting examples of virulence factors inhibited by an inventive compound of formula (I) or an inventive composition, preferably an inventive pharmaceutical composition, comprising an inventive compound of formula (I), as described herein, include one or more of toxins (e.g., α, β, γ, γ-variant, and δ-hemolysins, PSMs (e.g., PSMa), Panton-Valentine leukocidin (PVL), leukotoxin E and D (LukED), leukotoxin G and H (LukGH), enterotoxins (e.g., enterotoxin B), exfoliative toxin), proteases (e.g., serine proteases, metalloproteases and cysteine proteases), nuclease, lipase, coagulase, hyaluronidase, fibronectin-binding protein, clumping factor, pyrogenic toxin superantigen (e.g., TSST-1). In a preferred embodiment, the virulence factor inhibited is RNAIII and/or its downstream targets or PSMa.

Depending on the severity of the infection or if used in a preventive manner, the anti-virulence drugs can be administered alone or in combination with therapeutic agents such as antibiotics typically and preferably used for prevention and treatment of infections caused by bacteria such as Staphylococcus, primarily by Staphylococcus aureus (e.g., pneumonia, blood stream infections, acute skin and skin structure infections).

The anti-virulence drugs can be administered alone or in combination with therapeutic agents typically and preferably used for prevention and treatment of chronic inflammatory skin disease (e.g., atopic dermatitis, Netherton syndrome, psoriasis) which are exacerbated by bacteria such as Staphylococcus, primarily by Staphylococcus aureus.

The term “antibiotic”, as used herein, refers to an antimicrobial agent or anti-infective that kills bacteria (bactericidal antibiotics) or inhibits growth and/or metabolism of bacteria (bacteriostatic antibiotics). Antibiotics are well-known to the skilled person in the art, and specific and preferred examples thereof include penicillins, cephalosporins, polymyxins, rifamycins, lipiarmycins, quinolones, sulfonamides, macrolides, oxazolidinones, lincosamides and tetracyclines.

The term “treating”, “treatment” or “therapy” as used herein refers to means of obtaining a desired physiological effect. The effect may be therapeutic in terms of partially or completely curing a disease or a condition and/or symptoms attributed to the disease or the condition. The term refers to inhibiting the disease or condition, i.e. arresting its development; or ameliorating the disease or condition, i.e. causing regression of the disease or condition.

The term “prevention” as used herein refers to means of preventing or delaying the onset of disease or condition and/or symptoms attributed to the disease or condition.

As used herein, the terms “subject” or “animal” or “patient” or “mammal,” refers to any subject, particularly a mammalian subject, for whom diagnosis, prognosis, prophylaxis or therapy is desired, for example, a human or a domesticated mammal such as a dog, cat or horse or a food animal such as a cow or sheep or pig, preferably to a human. Thus, in a preferred embodiment of the present invention, said subject is a human.

As used herein, the term “for use” as used in “composition for use in treatment or prevention of a disease” shall disclose also the corresponding method of treatment or prevention and the corresponding use of a preparation for the manufacture of a medicament for the treatment or prevention of a disease”.

A “therapeutically effective amount” or “effective amount” is the amount of a compound or pharmaceutical composition in accordance with the present invention that will elicit the biological or medical response of a subject, preferably a human subject that is being sought by the researcher, veterinarian, medical doctor or other clinician. The term “therapeutic administration”, as used herein, should refer to the administration of therapeutically effective amount. In particular, the terms “effective”, “effective amount”, and “therapeutically effective amount”, as used herein, typically and preferably, refer to that amount of an inventive compounds of formula (I) or an inventive composition, preferably an inventive pharmaceutical composition, comprising an inventive compound of formula (I) that reduces the virulence of a bacterium or that results in amelioration of symptoms or a prolongation of survival in a subject with a bacteria related disease or disorder. The term “effective amount” is used generically herein to refer to the amount of a given compound or in case of a mixture the combined amount of mixture components that provides a measurable effect for a listed function. It will be understood by one of ordinary skill in the art, that for a given application, the effective amount can be determined by application of routine experimentation and without undue experimentation by methods that are described herein or that are known in the art. The term “therapeutically effective amount” is used generically herein to refer to the amount of a given compound or in case of a mixture the combined amount of mixture components when administered to the individual including a human or non-human animal, that provides a measurable therapeutic effect for a listed disease, disorder or condition to at least partially ameliorate a symptom of such disease, disorder or condition. The result of treatment can be partially or completely alleviating, inhibiting, preventing, ameliorating and/or relieving the disorder, condition or one or more symptoms thereof.

### Compounds of the Invention

In a first aspect, the present disclosure provides a compound of Formula (I), or a pharmaceutically acceptable salt, solvate, hydrate, or tautomer thereof:

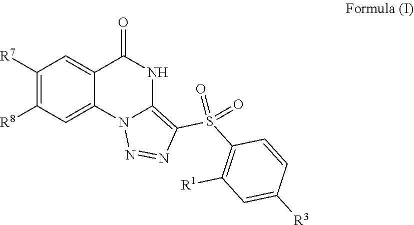

wherein:
R¹ is independently selected from —H, halogen, —C₁-C₆ alkyl, and —C₁-C₆ alkoxy, wherein said alkyl and alkoxy is each optionally substituted with one or more R¹¹;
R³ is independently selected from —H, —C₁-C₆ alkyl, and —C₁-C₆ alkoxy, wherein said alkyl and alkoxy is each optionally substituted by one or more R¹¹;
R⁷ is independently selected from —H and —OH;
R⁸ is a group of the Formula (A):

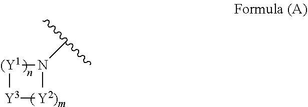

wherein n is independently an integer from 0 to 3;
m is independently an integer from 0 to 3;
Y¹ is independently, at each occurrence, selected from CR⁸¹R⁸², C(O), and O;
Y² is independently, at each occurrence, selected from CR^{81A}R^{82A}, and O;
Y³ is independently selected from CR⁸⁴R⁸⁵; CHR⁸⁶; NR⁸⁷, and O;
wherein the sum of m and n is at least 2 when Y³ is CR⁸⁴R⁸⁵ and wherein the sum of m and n is at least 3 when Y³ is CHR⁸⁶, NR⁸⁷, or O;
R⁸¹, R⁸², R^{81A} and R^{82A} are independently, at each occurrence, selected from —H and —C₁-C₆ alkyl, or one of R⁸¹ and R^{81A}, together with the carbon atoms to which they are attached, can combine to form a bridging ethylene;
R⁸⁴ and R⁸⁵, together with the carbon atom to which they are attached, combine to form a —C₄-C₇ cycloalkyl or a 4- to 7-membered heterocycle, wherein said cycloalkyl and heterocycle is each optionally substituted with one or more halogen, —C₁-C₆ alkyl, —C₁-C₆ haloalkyl, —OH, or —COOH;
R⁸⁶ is independently selected from —H, —COOH, and —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH;
or R⁸⁷, together with the nitrogen atom to which it is attached, can combine with one of R⁸¹, R^{81A}, R⁸² or R^{82A} and the carbon atoms to which they are attached to form a 5- to 6-membered heterocycle or 5- to 6-membered heteroaryl, wherein said heterocycle or heteroaryl is each optionally substituted with one or more halogen, —C₁-C₆ alkyl, —C₁-C₆ haloalkyl, or —OH; and
R¹¹ is independently, at each occurrence, halogen or —OH.

In one aspect, the present disclosure provides a compound of Formula (I), or a pharmaceutically acceptable salt, solvate, hydrate, or tautomer thereof:

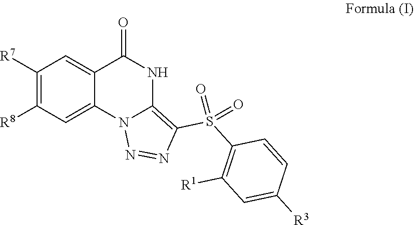

wherein:
R¹ is independently selected from —H, halogen, —C₁-C₆ alkyl, and —C₁-C₆ alkoxy, wherein said alkyl and alkoxy is each optionally substituted with one or more R¹¹;
R³ is independently selected from —H, —C₁-C₆ alkyl, and —C₁-C₆ alkoxy, wherein said alkyl and alkoxy is each optionally substituted by one or more R¹¹;
R⁷ is independently selected from —H and —OH;
R⁸ is a group of the Formula (A):

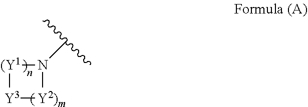

wherein n is independently an integer from 0 to 3;
m is independently an integer from 0 to 3;
Y¹ is independently, at each occurrence, selected from CR⁸¹R⁸², C(O), and O;
Y² is independently, at each occurrence, selected from CR^{81A}R^{82A}, and O;
Y³ is independently selected from CR⁸⁴R⁸⁵; CHR⁸⁶; NR⁸⁷, and O;
wherein the sum of m and n is at least 2 when Y³ is CR⁸⁴R⁸⁵ and wherein the sum of m and n is at least 3 when Y³ is CHR⁸⁶, NR⁸⁷, or O;
provided that the sum of m and n is 4, 5, or 6 when Y¹ is CR⁸¹R⁸², Y² is CR^{81A}R^{82A}. Y³ is CHR⁸⁶ and R⁸¹, R⁸², R^{81A}, R^{82A} and R⁸⁶ are each —H;
R⁸¹, R⁸², R^{81A} and R^{82A} are independently, at each occurrence, selected from —H and —C₁-C₆ alkyl, or one of R⁸¹ and R^{81A}, together with the carbon atoms to which they are attached, can combine to form a bridging ethylene;
R⁸⁴ and R⁸⁵, together with the carbon atom to which they are attached, combine to form a —C₄-C₇ cycloalkyl or a 4- to 7-membered heterocycle, wherein said cycloalkyl and heterocycle is each optionally substituted with one or more halogen, —C₁-C₆ alkyl, —C₁-C₆ haloalkyl, —OH, or —COOH;
R⁸⁶ is independently selected from —H, —COOH, and —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH;
or R⁸⁷, together with the nitrogen atom to which it is attached, can combine with one of R⁸¹, R^{81A}, R⁸² or R^{82A} and the carbon atoms to which they are attached to form a 5- to 6-membered heterocycle or 5- to 6-membered heteroaryl, wherein said heterocycle or heteroaryl is each optionally substituted with one or more halogen, —C₁-C₆ alkyl, —C₁-C₆ haloalkyl, or —OH; and R¹¹ is independently, at each occurrence, halogen or —OH.

In one aspect, the present disclosure provides a compound of Formula (I), or a pharmaceutically acceptable salt, solvate, hydrate, or tautomer thereof:

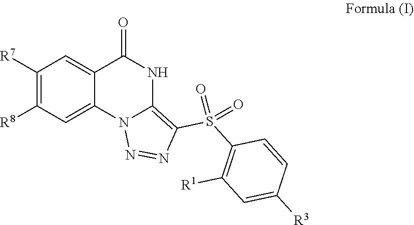

wherein:
R¹ is independently selected from —H, halogen, —C₁-C₆ alkyl, and —C₁-C₆ alkoxy, wherein said alkyl and alkoxy is each optionally substituted with one or more R¹¹;
R³ is independently selected from —H, —C₁-C₆ alkyl, and —C₁-C₆ alkoxy, wherein said alkyl and alkoxy is each optionally substituted by one or more R¹¹;
R⁷ is independently selected from —H and —OH;
R⁸ is a group of the Formula (A):

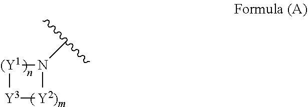

wherein n is independently an integer from 0 to 3;
m is independently an integer from 0 to 3;
Y¹ is independently, at each occurrence, selected from CR⁸¹R⁸², C(O), and O;
Y² is independently, at each occurrence, selected from CR^{81A}R^{82A}, and O;
Y³ is independently selected from CR⁸⁴R⁸⁵; CHR⁸⁶; NR⁸⁷, and O;
wherein the sum of m and n is at least 2 when Y³ is CR⁸⁴R⁸⁵ and wherein the sum of m and n is at least 3 when Y³ is CHR⁸⁶, NR⁸⁷, or O;
R⁸¹, R⁸², R^{81A} and R^{82A} are independently, at each occurrence, selected from —H and —C₁-C₆ alkyl, or one of R⁸¹ and R^{81A}, together with the carbon atoms to which they are attached, can combine to form a bridging ethylene;
R⁸⁴ and R⁸⁵, together with the carbon atom to which they are attached, combine to form a —C₄-C₇ cycloalkyl or a 4- to 7-membered heterocycle, wherein said cycloalkyl and heterocycle is each optionally substituted with one or more halogen, —C₁-C₆ alkyl, —C₁-C₆ haloalkyl, —OH, or —COOH;
R⁸⁶ is —COOH or —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH;
or R⁸⁷ together with the nitrogen atom to which it is attached, can combine with one of R⁸¹, R^{81A}, R⁸² or R^{82A} and the carbon atoms to which they are attached to form a 5- to 6-membered heterocycle or 5- to 6-membered heteroaryl, wherein said heterocycle or heteroaryl is each optionally substituted with one or more halogen, —C₁-C₆ alkyl, —C₁-C₆ haloalkyl, or —OH; and
R¹¹ is independently, at each occurrence, halogen or —OH.

In one aspect, the present disclosure provides a compound of Formula (I), or a pharmaceutically acceptable salt, solvate, hydrate, or tautomer thereof:

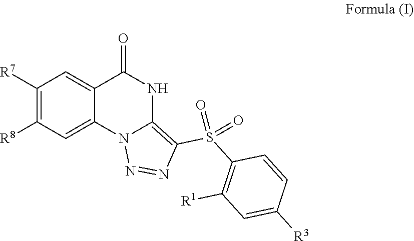

wherein:
R¹ is independently selected from —H, halogen, —C₁-C₆ alkyl, and —C₁-C₆ alkoxy, wherein said alkyl and alkoxy is each optionally substituted with one or more R¹¹;
R³ is independently selected from —H, —C₁-C₆ alkyl, and —C₁-C₆ alkoxy, wherein said alkyl and alkoxy is each optionally substituted by one or more R¹¹;
R⁷ is independently selected from —H and —OH;
R⁸ is a group of the Formula (A):

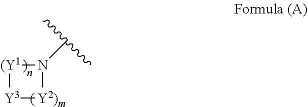

wherein n is independently an integer from 0 to 3;
m is independently an integer from 0 to 3;
Y¹ is independently, at each occurrence, selected from CR⁸¹R⁸², C(O), and O;
Y² is independently, at each occurrence, selected from CR^{81A}R^{82A}, and O;
Y³ is independently selected from CR⁸⁴R⁸⁵; CHR⁸⁶; NR⁸⁷, and O;
wherein the sum of m and n is at least 2 when Y³ is CR⁸⁴R⁸⁵ and wherein the sum of m and n is at least 3 when Y³ is CHR⁸⁶, NR⁸⁷, or O;
R⁸¹, R⁸², R^{81A} and R^{82A} are independently, at each occurrence, selected from —H and —C₁—C₆ alkyl, or one of R⁸¹ and R^{81A}, together with the carbon atoms to which they are attached, can combine to form a bridging ethylene;
R⁸⁴ and R⁸⁵, together with the carbon atom to which they are attached, combine to form a —C₄-C₇ cycloalkyl or a 4- to 7-membered heterocycle, wherein said cycloalkyl and heterocycle is each optionally substituted with one or more halogen, —C₁-C₆ alkyl, —C₁-C₆ haloalkyl, or —OH;
R⁸⁶ is independently selected from —H, and —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH;
or R⁸⁷, together with the nitrogen atom to which it is attached, can combine with one of R⁸¹, R^{81A}, R⁸² or R^{82A} and the carbon atoms to which they are attached to form a 5- to 6-membered heterocycle or 5- to 6-membered heteroaryl, wherein said heterocycle or heteroaryl is each optionally substituted with one or more halogen, —C₁-C₆ alkyl, —C₁-C₆ haloalkyl, or —OH; and
R¹¹ is independently, at each occurrence, halogen or —OH.

In some embodiments, R¹ is independently selected from —H, halogen, —C₁-C₄ alkyl, and —C₁-C₄ alkoxy, wherein said alkyl and alkoxy is each optionally substituted with one or more R¹¹.

In some embodiments, R¹ is independently selected from —H, —F, —Cl, —C₁-C₄ alkyl, and —C₁-C₄ alkoxy, wherein said alkyl and alkoxy is each optionally substituted with one or more F, Cl, and —OH.

In some embodiments, R¹ is independently selected from —H, —F, —C₁-C₂ alkyl, and —C₁—C₂ alkoxy, wherein said alkyl and alkoxy is each optionally substituted with one or more —F and —OH.

In some embodiments, R¹ is independently selected from —H, —F, and —C₁-C₂ alkyl, wherein said alkyl is optionally substituted with one or more —F and —OH.

In some embodiments, R¹ is independently selected from —H, —F, and —C₁-C₂ alkyl.

In some embodiments, R¹ is independently selected from —H and —C₁-C₂ alkyl.

In some embodiments, R¹ is —CH₃.

In some embodiments, R³ is independently selected from —H, —C₁-C₄ alkyl and —C₁-C₄ alkoxy, wherein said alkyl and alkoxy is each optionally substituted by one or more R¹¹.

In some embodiments, R³ is independently selected from —H, —C₁-C₄ alkyl, and —C₁-C₄ alkoxy, wherein said alkyl and alkoxy is each optionally substituted by one or more —F, —Cl, or —OH.

In some embodiments, R³ is independently selected from —H and —C₁-C₄ alkyl, wherein said alkyl is optionally substituted by one or more —F, —Cl, or —OH.

In some embodiments, R³ is independently selected from —H and —C₁-C₄ alkyl.

In some embodiments, R³ is independently selected from —H and —C₁-C₂ alkyl.

In some embodiments, R³ is independently —C₁-C₂ alkyl.

In some embodiments, R³ is —CH₃. In some embodiments, R¹ and R³ are —CH₃.

In some embodiments, R⁷ is —H. In some embodiments, R⁷ is —OH.

In some embodiments, Y³ is independently selected from CR⁸⁴R⁸⁵; CHR⁸⁶; NR⁸⁷, and 0;
wherein the sum of m and n is at least 2 when Y³ is CR⁸⁴R⁸⁵ and wherein the sum of m and n is at least 3 when Y³ is CHR⁸⁶, NR⁸⁷, or O;
provided that the sum of m and n is 4, 5, or 6 when Y¹ is CR⁸¹R⁸², Y² is CR^{81A}R^{82A}, Y³ is CHR⁸⁶ and R⁸¹, R⁸², R^{81A}, R^{82A} and R⁸⁶ are each —H.

In some embodiments, Y¹ is independently, at each occurrence, selected from CR⁸¹R⁸²; Y² is independently, at each occurrence, selected from CR^{81A}R^{82A}; and Y³ is independently selected from CR⁸⁴R⁸⁵; CHR⁸⁶; and NR⁸⁷.

In some embodiments, Y³ is CR⁸⁴R⁸⁵.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₆ alkyl, —C₁-C₆ haloalkyl, —OH or —COOH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₆ alkyl, —C₁-C₆ haloalkyl, or —OH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₃ alkyl, —C₁-C₃ haloalkyl, —OH or —COOH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₃ alkyl, —C₁-C₃ haloalkyl, or —OH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more —C₁-C₃ alkyl, —OH or —COOH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more —C₁-C₃ alkyl or —OH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or more heteroatoms selected from O and N; and wherein said cycloalkyl or heterocycle is each optionally substituted with one or more —C₁-C₃ alkyl, —OH or —COOH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or more heteroatoms selected from O and N; and wherein said cycloalkyl or heterocycle is each optionally substituted with one or more —C₁-C₃ alkyl or —OH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₃ alkyl, —OH or —COOH.

In some embodiments, Y³ is CR⁸⁴R⁸⁵; R¹⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₃ alkyl or —OH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₂ alkyl, —OH or —COOH, preferably wherein said cycloalkyl or heterocycle is each optionally substituted with one or two —CH₃ or —OH.

In some embodiments, Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₂ alkyl or —OH, preferably wherein said cycloalkyl or heterocycle is each optionally substituted with one or two —CH₃ or —OH.

In some embodiments, Y³ is CR⁸⁴R⁸⁵; R⁸⁴ and R⁸⁵, together with the carbon atom to which they are attached, combine to form a —C₄ cycloalkyl, a —C₄ heterocycle, or a —C₆ heterocycle, wherein said C₄ and C₆ heterocycle each comprises one or two heteroatoms selected from O and N; and wherein said cycloalkyl, C₄ heterocycle and C₆ heterocycle is each optionally substituted with one or more —C₁-C₂ alkyl, —OH or —COOH, preferably wherein each cycloalkyl, C₄ heterocycle and C₆ heterocycle is each optionally substituted with one or two —CH₃ or —OH.

In some embodiments, Y³ is CR⁸⁴R⁸⁵; R⁸⁴ and R⁸⁵, together with the carbon atom to which they are attached, combine to form a —C₄ cycloalkyl, a —C₄ heterocycle, or a —C₆ heterocycle, wherein said C₄ and C₆ heterocycle each comprises one or two heteroatoms selected from O and N; and wherein said cycloalkyl, C₄ heterocycle and C₆ heterocycle is each optionally substituted with one or more —C₁-C₂ alkyl or —OH, preferably wherein each cycloalkyl, C₄ heterocycle and C₆ heterocycle is each optionally substituted with one or two —CH₃ or —OH.

In some embodiments, Y³ is CR⁸⁴R⁸⁵; R⁸⁴ and R⁸⁵, together with the carbon atom to which they are attached, combine to form a cyclobutane, azetidine, oxetane, piperidine, or morpholine; and wherein said cyclobutane, azetidine, oxetane, piperidine, or morpholine is each optionally substituted with one or more —C₁-C₂ alkyl, —OH or —COOH, preferably wherein said cyclobutane, azetidine, oxetane, piperidine, or morpholine is each optionally substituted with one or two —CH₃ or —OH.

In some embodiments, Y³ is CR⁸⁴R⁸⁵; R⁸⁴ and R⁸⁵, together with the carbon atom to which they are attached, combine to form a cyclobutane, azetidine, oxetane, piperidine, or morpholine; and wherein said cyclobutane, azetidine, oxetane, piperidine, or morpholine is each optionally substituted with one or more —C₁-C₂ alkyl or —OH, preferably wherein said cyclobutane, azetidine, oxetane, piperidine, or morpholine is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₆ alkyl, —C₁-C₆ haloalkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₆ alkyl, —C₁-C₆ haloalkyl, or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₃ alkyl, —C₁-C₃ haloalkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₃ alkyl, —C₁-C₃ haloalkyl, or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more —C₁-C₃ alkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more —C₁-C₃ alkyl or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises heteroatoms selected from O and N; and wherein said cycloalkyl or heterocycle is each optionally substituted with one or more —C₁-C₃ alkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises heteroatoms selected from O and N; and wherein said cycloalkyl or heterocycle is each optionally substituted with one or more —C₁-C₃ alkyl or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₃ alkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₃ alkyl or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₂ alkyl, —OH or —COOH, preferably wherein said cycloalkyl or heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₂ alkyl or —OH, preferably wherein said cycloalkyl or heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄ cycloalkyl, a —C₄ heterocycle, or a —C₆ heterocycle, wherein said C₄ heterocycle and C₆ heterocycle each comprises one or two heteroatoms selected from O and N; and wherein said cycloalkyl, C₄ heterocycle and C₆ heterocycle is each optionally substituted with one or more —C₁-C₂ alkyl, —OH or —COOH, preferably wherein said cycloalkyl, C₄ heterocycle and C₆ heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a —C₄ cycloalkyl, a —C₄ heterocycle, or a —C₆ heterocycle, wherein said C₄ heterocycle and C₆ heterocycle each comprises one or two heteroatoms selected from O and N; and wherein said cycloalkyl, C₄ heterocycle and C₆ heterocycle is each optionally substituted with one or more —C₁-C₂ alkyl or —OH, preferably wherein said cycloalkyl, C₄ heterocycle and C₆ heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a cyclobutane, azetidine, oxetane, piperidine, or morpholine; and wherein said cyclobutane, azetidine, oxetane, piperidine, or morpholine is each optionally substituted with one or more —C₁-C₂ alkyl, —OH or —COOH, preferably wherein said cyclobutane, azetidine, oxetane, piperidine, or morpholine is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; Y³ is CR⁸⁴R⁸⁵; R⁸⁴ and R⁸⁵, together with the carbon atom to which they are attached, combine to form a cyclobutane, azetidine, oxetane, piperidine, or morpholine; and wherein said cyclobutane, azetidine, oxetane, piperidine, or morpholine is each optionally substituted with one or more —C₁-C₂ alkyl or —OH, preferably wherein said cyclobutane, azetidine, oxetane, piperidine, or morpholine is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₆ alkyl, —C₁-C₆ haloalkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₆ alkyl, —C₁-C₆ haloalkyl, or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₃ alkyl, —C₁-C₃ haloalkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more halogen, —C₁-C₃ alkyl, —C₁-C₃ haloalkyl, or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more —C₁-C₃ alkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said cycloalkyl or heterocycle is each optionally substituted with one or more —C₁-C₃ alkyl or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises heteroatoms selected from O and N; and wherein said cycloalkyl or heterocycle is each optionally substituted with one or more —C₁-C₃ alkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises heteroatoms selected from O and N; and wherein said cycloalkyl or heterocycle is each optionally substituted with one or more —C₁-C₃ alkyl or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₃ alkyl, —OH or —COOH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₃ alkyl or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₂ alkyl, —OH or —COOH, preferably wherein said cycloalkyl or heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄-C₆ cycloalkyl or 4- to 6-membered heterocycle, wherein said heterocycle comprises one or two heteroatoms selected from O and N; and wherein said cycloalkyl or heterocycle is each optionally substituted with one or more —C₁-C₂ alkyl or —OH, preferably wherein said cycloalkyl or heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄ cycloalkyl, a —C₄ heterocycle, or a —C₆ heterocycle, wherein said C₄ heterocycle and C₆ heterocycle each comprises one or two heteroatoms selected from O and N; and wherein said cycloalkyl, C₄ heterocycle, and C₆ heterocycle is each optionally substituted with one or more —C₁-C₂ alkyl, —OH or —COOH, preferably wherein said cycloalkyl, C₄ heterocycle, and C₆ heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a —C₄ cycloalkyl, a —C₄ heterocycle, or a —C₆ heterocycle, wherein said C₄ heterocycle and C₆ heterocycle each comprises one or two heteroatoms selected from O and N; and wherein said cycloalkyl, C₄ heterocycle, and C₆ heterocycle is each optionally substituted with one or more —C₁-C₂ alkyl or —OH, preferably wherein said cycloalkyl, C₄ heterocycle, and C₆ heterocycle is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a cyclobutane, azetidine, oxetane, piperidine, or morpholine; and wherein said cyclobutane, azetidine, oxetane, piperidine, or morpholine is each optionally substituted with one or more —C₁-C₂ alkyl, —OH or —COOH, preferably wherein said cyclobutane, azetidine, oxetane, piperidine, or morpholine is each optionally substituted with one or two —CH₃ or —OH.

In some embodiments, Y¹ is independently, at each occurrence, CR⁸¹R⁸² or O, preferably wherein Y¹ is independently at each occurrence CR⁸¹R⁸²; Y² is independently, at each occurrence, CR^{81A}R^{82A} or O, preferably wherein Y² is independently at each occurrence CR^{81A}R^{82A}; R⁸¹ and R^{81A} are independently, at each occurrence, H; R⁸² and R^{82A} are each independently, at each occurrence, H or —C₁-C₃ alkyl, preferably wherein R⁸² and R^{82A} are independently at each occurrence H; m and n are each independently 0, 1, 2 or 3, wherein the sum of m and n is 2, 3 or 4; Y³ is CR⁸⁴R⁸⁵; R⁸⁴ and R⁸⁵, together with the carbon atom to which they are attached, combine to form a cyclobutane, azetidine, oxetane, piperidine, or morpholine; and wherein said cyclobutane, azetidine, oxetane, piperidine, or morpholine is each optionally substituted with one or more —C₁-C₂ alkyl or —OH, preferably wherein said cyclobutane, azetidine, oxetane, piperidine, or morpholine is each optionally substituted with one or two —CH₃ or —OH.

In some embodiments, Y³ is CHR⁸⁶.

In some embodiments, Y³ is CHR⁸⁶; R⁸⁶ is independently selected from —H, —COOH, and —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
provided that the sum of m and n is 4, 5, or 6 when Y¹ is CR⁸¹R⁸², Y² is CR^{81A}R^{82A}, Y³ is CHR⁸⁶ and R⁸¹, R⁸², R^{81A}, R^{82A} and R⁸⁶ are each —H.

In some embodiments, Y³ is CHR⁸⁶ and R⁸⁶ is —H or —COOH.

In some embodiments, Y³ is CHR⁸⁶ and R⁸⁶ is H.

In some embodiments, Y³ is CHR⁸⁶ and R⁸⁶ is —COOH.

In some embodiments, Y³ is CHR⁸⁶ and R⁸⁶ is H, provided that the sum of m and n is 4, 5 or 6 when Y¹ is CR⁸¹R⁸², Y² is CR^{81A}R^{82A}, and R⁸¹, R⁸², R^{81A}, R^{82A} are each —H.

In some embodiments, Y³ is CHR⁸⁶; R⁸⁶ is H; and the sum of m and n is 3 or 4.

In some embodiments, Y³ is CHR⁸⁶; R⁸⁶ is H; and the sum of m and n is 4, 5 or 6.

In some embodiments, Y³ is CHR⁸⁶; R⁸⁶ is H; and the sum of m and n is 4.

In some embodiments, Y³ is CHR⁸⁶; R⁸⁶ is H; and the sum of m and n is 5.

In some embodiments, Y³ is CHR⁸⁶; R⁸⁶ is H; and the sum of m and n is 6.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; and the sum of m and n is 3 or 4.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; and the sum of m and n is 4, 5, or 6.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; and the sum of m and n is 4.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; and the sum of m and n is 5.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; and the sum of m and n is 6.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; the sum of m and n is 3 or 4; and R⁸¹, R⁸², R^{81A} and R^{82A} are independently, at each occurrence, selected from —H and —C₁-C₂ alkyl, preferably wherein R⁸¹, R⁸², R^{81A} and R^{82A} are independently, at each occurrence, H.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; the sum of m and n is 4, 5, or 6; and R⁸¹, R⁸². R^{81A} and R^{82A} are independently, at each occurrence, selected from —H and —C₁-C₂ alkyl, preferably wherein R⁸¹, R⁸², R^{81A} and R^{82A} are independently, at each occurrence, H.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; the sum of m and n is 4; and R⁸¹, R⁸², R^{81A} and R^{82A} are independently, at each occurrence, selected from —H and —C₁-C₂ alkyl, preferably wherein R⁸¹, R⁸², R^{81A} and R^{82A} are independently, at each occurrence, H.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; the sum of m and n is 5; and R⁸¹, R⁸², R^{81A} and R^{82A} are independently, at each occurrence, selected from —H and —C₁-C₂ alkyl, preferably wherein R⁸¹, R⁸², R^{81A} and R^{82A} are independently, at each occurrence, H.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is H; the sum of m and n is 6; and R⁸¹, R⁸², R^{81A} and R^{82A} are independently, at each occurrence, selected from —H and —C₁-C₂ alkyl, preferably wherein R⁸¹, R⁸², R^{81A} and R^{82A} are independently, at each occurrence, H.

In some embodiments, R⁸⁶ is —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle.

In some embodiments, R⁸⁶ is —COOH or —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle.

In some embodiments, Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH.

In some embodiments, Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH.

In some embodiments, Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more —C₁-C₆ alkyl or —OH, preferably —C₁-C₂ alkyl or —OH.

In some embodiments, Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more —C₁-C₆ alkyl or —OH, preferably —C₁-C₂ alkyl or —OH.

In some embodiments, Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle.

In some embodiments, Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle.

In some embodiments, Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or more heteroatoms selected from O and N.

In some embodiments, Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or more heteroatoms selected from O and N.

In some embodiments, Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or two heteroatoms selected from O and N.

In some embodiments, Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or two heteroatoms selected from O and N.

In some embodiments, Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a pyrrolidine.

In some embodiments, Y³ is CHR⁸⁶ and R⁸⁶, together with the carbon atom to which it is attached, combines with one of R⁸¹, R^{81A}, R⁸² or R^{82A} and the carbon atoms to which they are attached to form a pyrrolidine.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, or —OH.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more —C₁-C₆ alkyl or —OH, preferably —C₁-C₂ alkyl or —OH.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is optionally substituted with one or more —C₁-C₆ alkyl or —OH, preferably —C₁-C₂ alkyl or —OH.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or more heteroatoms selected from O and N.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or more heteroatoms selected from O and N.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or two heteroatoms selected from O and N.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle comprises one or two heteroatoms selected from O and N.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶, together with the carbon atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶ is —COOH or R⁸⁶, together with the carbon atom to which it is attached, combines with one of R⁸¹, R^{81A}, R⁸² or R^{82A} and the carbon atoms to which they are attached to form a pyrrolidine.

In some embodiments, Y¹ is independently, at each occurrence, CR⁸¹R⁸²; Y² is independently, at each occurrence, CR^{81A}R^{82A}; the sum of m and n is 2, 3 or 4, preferably wherein the sum of m and n is 3; Y³ is CHR⁸⁶ and R⁸⁶, together with the carbon atom to which it is attached, combines with one of R⁸¹, R^{81A}, R⁸² or R^{82A} and the carbon atoms to which they are attached to form a pyrrolidine.

In some embodiments, Y³ is CHR⁸⁶ and R⁸⁶ is —COOH or NR⁸⁸R⁸⁹.

In some embodiments, Y³ is CHR⁸⁶ and R⁸⁶ is NR⁸⁸R⁸⁹.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a pyrrolidine.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a pyrrolidine.

In some embodiments, Y³ is CHR⁸⁶, R⁸⁶ is —COOH or NR⁸⁸R⁸⁹, and m and n are each independently 1 or 2, preferably wherein m is 2 and n is 1 or 2.

In some embodiments, Y³ is CHR⁸⁶, R⁸⁶ is NR⁸⁸R⁸⁹, and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶, R⁸⁶ is —COOH or NR⁸⁸R⁸⁹, and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶, R⁸⁶ is NR⁸⁸R⁸⁹, and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; and R⁸⁸ and R⁸⁹ are independently, at each occurrence, selected from —H and —C₁-C₃ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; and R⁸⁸ and R⁸⁹ are independently, at each occurrence, selected from —H and —C₁-C₂ alkyl; or one of R⁸⁸ and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₃ alkyl.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —CH₃.

In some embodiments, Y³ is CHR⁸⁶; R⁸⁶ is NR⁸⁸R⁸⁹; and R⁸⁸ and R⁸⁹ are independently, at each occurrence, selected from —H and —CH₃.

In some embodiments, Y³ is CHR⁸⁶; R⁸⁶ is-COOH or NR⁸⁸R⁸⁹; and R⁸⁸ and R⁸⁹ are independently, at each occurrence, selected from —H and —C₁-C₃ alkyl; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₃ alkyl; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —C₁-C₂ alkyl; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; and R⁸⁸ and R⁸⁹ are independently, at each occurrence, selected from —H and —CH₃; and m and n are each independently 1 or 2, preferably wherein m is 2 and n is 1 or 2.

In some embodiments, Y³ is CHR⁸⁶; R⁸⁶ is NR⁸⁸R⁸⁹; and R⁸⁸ and R⁸⁹ are independently, at each occurrence, selected from —H and —CH₃; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; R⁸¹, R^{81A}, R⁸² and R⁸² are each independently, at each occurrence, selected from —C₁-C₂ alkyl and —H, preferably wherein R⁸¹, R^{81A}, R⁸² and R⁸² are each independently, at each occurrence, H; Y³ is CHR⁸⁶; R⁸⁶ is —COOH or NR⁸⁸R⁸⁹; and R⁸⁸ and R⁸⁹ are independently, at each occurrence, selected from —H and —C₁-C₃ alkyl; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; R⁸¹, R^{81A}, R⁸² and R⁸² are each independently, at each occurrence, selected from —C₁-C₂ alkyl and —H, preferably wherein R⁸¹, R^{81A}, R⁸² and R⁸² are each independently, at each occurrence, H; Y³ is CHR⁸⁶; R⁸⁶ is NR⁸⁸R⁸⁹; and R⁸⁸ and R⁸⁹ are independently, at each occurrence, selected from —H and —C₁-C₃ alkyl; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; R⁸¹, R^{81A}, R⁸² and R⁸² are each independently, at each occurrence, selected from —C₁-C₂ alkyl and —H, preferably wherein R⁸¹, R^{81A}, R⁸² and R⁸² are each independently, at each occurrence, H; Y³ is CHR⁸⁶; R⁸⁶ is —COOH or NR⁸⁸R⁸⁹; and R⁸⁸ and R⁸⁹ are independently, at each occurrence, selected from —H and —C₁-C₂ alkyl; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; R⁸¹, R^{81A}, R⁸² and R⁸² are each independently, at each occurrence, selected from —C₁-C₂ alkyl and —H, preferably wherein R⁸¹, R^{81A}, R⁸² and R⁸² are each independently, at each occurrence, H; Y³ is CHR⁸⁶; R⁸⁶ is NR⁸⁸R⁸⁹; and R⁸⁸ and R⁸⁹ are independently, at each occurrence, selected from —H and —C₁-C₂ alkyl; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; R⁸¹, R^{81A}, R⁸² and R⁸² are each independently, at each occurrence, selected from —C₁-C₂ alkyl and —H, preferably wherein R⁸¹, R^{81A}, R⁸² and R⁸² are each independently, at each occurrence, H; Y³ is CHR⁸⁶; R⁸⁶ is —COOH or NR⁸⁸R⁸⁹; and R⁸⁸ and R⁸⁹ are independently, at each occurrence, selected from —H and —CH₃; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; R⁸¹, R^{81A}, R⁸² and R⁸² are each independently, at each occurrence, selected from —C₁-C₂ alkyl and —H, preferably wherein R⁸¹, R^{81A}, R⁸² and R⁸² are each independently, at each occurrence, H; Y³ is CHR⁸⁶; R⁸⁶ is NR⁸⁸R⁸⁹; and R⁸⁸ and R⁸⁹ are independently, at each occurrence, selected from —H and —CH₃; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a pyrrolidine.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a pyrrolidine.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or more atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle comprises one or two atoms selected from O and N; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a 5- to 6-membered heterocycle, wherein said heterocycle is selected from piperidine, morpholine, piperazine, and pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is —COOH or NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y¹ is independently, at each occurrence, CR⁸¹R⁸²; Y² is independently, at each occurrence, CR^{81A}R^{82A}; Y³ is CHR⁸⁶; R⁸⁶ is NR⁸⁸R⁸⁹; R⁸⁸ is independently —H or —C₁-C₂ alkyl; and R⁸⁹, together with the nitrogen atom to which it is attached, combines with one of R⁸¹, R^{81A}, R⁸² or R^{82A} and the carbon atoms to which they are attached to form a pyrrolidine; and m and n are each independently 1 or 2, preferably wherein m is 2 and n is 1 or 2.

In some embodiments, Y³ is NR⁸⁷.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; and wherein said 4- to 6-membered heterocycle is optionally substituted with one or more halogen, —C₁-C₆ alkyl, —C₁-C₆ haloalkyl, or —OH; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl is each optionally substituted with one or more halogen, —C₁-C₆ alkyl, —C₁-C₆ haloalkyl, or —OH.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more —C₁-C₆ alkyl, or —OH; and wherein said 4- to 6-membered heterocycle is optionally substituted with one or more —C₁-C₆ alkyl, or —OH; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl is each optionally substituted with one or more —C₁-C₆ alkyl, -or —OH.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; wherein said cycloalkyl is optionally substituted with one or more —C₁-C₆ alkyl, or —OH; and wherein said 4- to 6-membered heterocycle is optionally substituted with one or more —C₁-C₆ alkyl, or —OH; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; and wherein said cycloalkyl or 4- to 6-membered heterocycle is each optionally substituted with one or more —C₁-C₆ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl each comprises one or more atoms selected from O and N.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₄ alkyl, —C₃-C₆ cycloalkyl, and 4- to 6-membered heterocycle; wherein each alkyl is optionally substituted with one or more —C₁-C₄ alkoxy, —OH, —NH₂, —NHC₁-C₄ alkyl, or N(C₁-C₄ alkyl)₂; and wherein said cycloalkyl or 4- to 6-membered heterocycle is each optionally substituted with one or more —C₁-C₄ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl each comprises one or two atoms selected from O and N.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle; wherein each alkyl is optionally substituted with one or more —C₁-C₂ alkoxy, —OH, —NH₂, —NHC₁-C₂ alkyl, or N(C₁-C₂ alkyl)₂; and wherein said cycloalkyl or 4- to 6-membered heterocycle is each optionally substituted with one or more —C₁-C₂ alkyl; or R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle comprises one or more atoms selected from O and N; wherein each alkyl is optionally substituted with one or more —C₁-C₂ alkoxy, —OH, —NH₂, —NHC₁-C₂ alkyl, or N(C₁-C₂ alkyl)₂; and wherein said 4- to 6-membered heterocycle is optionally substituted with one or more —C₁-C₂ alkyl; or R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle comprises one or two atoms selected from O and N; wherein each alkyl is optionally substituted with one or more —C₁-C₂ alkoxy, —OH, —NH₂, —NHC₁-C₂ alkyl, or N(C₁-C₂ alkyl)₂; and wherein said 4- to 6-membered heterocycle is optionally substituted with one or more —C₁-C₂ alkyl; or R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle is selected from piperidine, piperazine, morpholine, pyrrolidine, tetrahydrofuran, azetidine, and oxetane; wherein each alkyl is optionally substituted with one or more —C₁-C₂ alkoxy, —OH, —NH₂, —NHC₁-C₂ alkyl, or N(C₁-C₂ alkyl)₂; and wherein said 4- to 6-membered heterocycle is optionally substituted with one or more —C₁-C₂ alkyl; or R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said 4- to 6-membered heterocycle is optionally substituted with one or more halogen, —C₁-C₆ alkyl, —C₁-C₆ haloalkyl, or —OH; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₆ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl is each optionally substituted with one or more halogen, —C₁-C₆ alkyl, —C₁-C₆ haloalkyl, or —OH.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; wherein said cycloalkyl is optionally substituted with one or more —C₁-C₆ alkyl, or —OH; wherein said 4- to 6-membered heterocycle is optionally substituted with one or more —C₁-C₆ alkyl, or —OH; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₆ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl is each optionally substituted with one or more —C₁-C₆ alkyl, or —OH.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; wherein said cycloalkyl is optionally substituted with one or more —C₁-C₆ alkyl, or —OH; and wherein said 4- to 6-membered heterocycle is optionally substituted with one or more —C₁-C₆ alkyl, or —OH; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₆ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; wherein said cycloalkyl or 4- to 6-membered heterocycle is each optionally substituted with one or more —C₁-C₆ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₆ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₄ alkyl, —C₃-C₆ cycloalkyl, and 4- to 6-membered heterocycle; wherein each alkyl is optionally substituted with one or more —C₁-C₄ alkoxy, —OH, —NH₂, —NHC₁-C₄ alkyl, or N(C₁-C₄ alkyl)₂; wherein said cycloalkyl or 4- to 6-membered heterocycle is each optionally substituted with one or more —C₁-C₄ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₄ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl each comprises one or more atoms selected from O and N.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle; wherein each alkyl is optionally substituted with one or more —C₁-C₂ alkoxy, —OH, —NH₂, —NHC₁-C₂ alkyl, or N(C₁-C₂ alkyl)₂; wherein said cycloalkyl or 4- to 6-membered heterocycle is each optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl; or R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said 5- to 6-membered heterocycle or 5- to 6-membered heteroaryl each comprises one or two atoms selected from O and N.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said 4- to 6-membered heterocycle comprises one or more atoms selected from O and N; wherein each alkyl is optionally substituted with one or more —C₁-C₂ alkoxy, —OH, —NH₂, —NHC₁-C₂ alkyl, or N(C₁-C₂ alkyl)₂; wherein said 4- to 6-membered heterocycle is optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl; or R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said 4- to 6-membered heterocycle comprises one or two atoms selected from O and N; wherein each alkyl is optionally substituted with one or more —C₁-C₂ alkoxy, —OH, —NH₂, —NHC₁-C₂ alkyl, or N(C₁-C₂ alkyl)₂; wherein said 4- to 6-membered heterocycle is optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl; or R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said 4- to 6-membered heterocycle is selected from piperidine, piperazine, morpholine, pyrrolidine, tetrahydrofuran, azetidine, and oxetane; wherein each alkyl is optionally substituted with one or more —C₁-C₂ alkoxy, —OH, —NH₂, —NHC₁-C₂ alkyl, or N(C₁-C₂ alkyl)₂; wherein said 4- to 6-membered heterocycle is optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl; or R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments, Y³ is NR⁸⁷ and R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said heterocycle or heteroaryl is each optionally substituted with one or more halogen, —C₁-C₆ alkyl, —C₁-C₆ haloalkyl, or —OH.

In some embodiments, Y³ is NR⁸⁷ and R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said heterocycle or heteroaryl is each optionally substituted with one or more —C₁-C₆ alkyl or —OH, preferably —C₁-C₂ alkyl or —OH.

In some embodiments, Y³ is NR⁸⁷ and R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl.

In some embodiments, Y³ is NR⁸⁷ and R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said heterocycle or heteroaryl comprises one or more atoms selected from O and N.

In some embodiments, Y³ is NR⁸⁷ and R⁸⁷, together with the nitrogen atom to which it is attached, combines with one of R⁸¹, R^{81A}, R⁸² or R^{82A} and the carbon atoms to which they are attached to form a 5- to 6-membered heterocycle or a 5- to 6-membered heteroaryl, wherein said heterocycle or heteroaryl comprises one or two atoms selected from O and N.

In some embodiments, Y³ is NR⁸⁷ and R⁸⁷, together with the nitrogen atom to which it is attached, combines with one of R⁸¹, R^{81A}, R⁸² or R^{82A} and the carbon atoms to which they are attached to form a piperidine, morpholine, piperazine, pyrrolidine, or imidazole.

In some embodiments wherein Y³ is NR⁸⁷, R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; and wherein said heterocycle is optionally substituted with one or more halogen, —C₁-C₆ alkyl, —C₁-C₆ haloalkyl, or —OH.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; wherein said cycloalkyl is optionally substituted with one or more —C₁-C₆ alkyl, or —OH; and wherein said heterocycle is optionally substituted with one or more halogen, —C₁-C₆ alkyl, or —OH.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; and wherein said cycloalkyl or heterocycle is each optionally substituted with one or more —C₁-C₆ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₄ alkyl, —C₃-C₆ cycloalkyl, and 4- to 6-membered heterocycle; wherein each alkyl is optionally substituted with one or more —C₁-C₄ alkoxy, —OH, —NH₂, —NHC₁-C₄ alkyl, or N(C₁-C₄ alkyl)₂; and wherein said cycloalkyl or heterocycle is each optionally substituted with one or more —C₁-C₄ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle; wherein each alkyl is optionally substituted with one or more —C₁-C₂ alkoxy, —OH, —NH₂, —NHC₁-C₂ alkyl, or N(C₁-C₂ alkyl)₂; and wherein said cycloalkyl or heterocycle is each optionally substituted with one or more —C₁-C₂ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle comprises one or more atoms selected from O and N; wherein each alkyl is optionally substituted with one or more —C₁-C₂ alkoxy, —OH, —NH₂, —NHC₁-C₂ alkyl, or N(C₁-C₂ alkyl)₂; and wherein said heterocycle is optionally substituted with one or more —C₁-C₂ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle comprises one or two atoms selected from O and N; wherein each alkyl is optionally substituted with one or more —C₁-C₂ alkoxy, —OH, —NH₂, —NHC₁-C₂ alkyl, or N(C₁-C₂ alkyl)₂; and wherein said heterocycle is optionally substituted with one or more —C₁-C₂ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle is selected from piperidine, piperazine, morpholine, tetrahydropyran, pyrrolidine, tetrahydrofuran, azetidine, and oxetane; wherein each alkyl is optionally substituted with one or more —C₁-C₂ alkoxy, —OH, —NH₂, —NHC₁-C₂ alkyl, or N(C₁-C₂ alkyl)₂; and wherein said heterocycle is optionally substituted with one or more —C₁-C₂ alkyl.

In some embodiments, Y³ is NR⁸⁷ and R⁸⁷ is independently selected from —H, —C₁-C₄ alkyl, and 4- to 6-membered heterocycle, wherein said heterocycle is selected from piperidine, piperazine, morpholine, tetrahydropyran, pyrrolidine, tetrahydrofuran, azetidine, and oxetane; wherein each alkyl is optionally substituted with one or more —C₁-C₂ alkoxy, —OH, —NH₂, —NHC₁-C₂ alkyl, or N(C₁-C₂ alkyl)₂; and wherein said heterocycle is optionally substituted with one or more —C₁-C₂ alkyl.

In some embodiments, Y³ is NR⁸⁷ and R⁸⁷ is independently selected from —H, —C₁-C₂ alkyl, and 4- to 6-membered heterocycle, wherein said heterocycle is selected from piperidine, piperazine, morpholine, tetrahydropyran, pyrrolidine, tetrahydrofuran, azetidine, and oxetane; and wherein said heterocycle is optionally substituted with one or more —C₁-C₂ alkyl.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₆ alkyl.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; wherein said cycloalkyl is optionally substituted with one or more —C₁-C₆ alkyl, or —OH; wherein said heterocycle is optionally substituted with one or more —C₁-C₆ alkyl, or —OH; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₆ alkyl.

In some embodiments, Y³ is NR⁸⁷ and R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more —C₁-C₆ alkoxy, —OH, —NH₂, —NHC₁-C₆ alkyl, or N(C₁-C₆ alkyl)₂; wherein said cycloalkyl or heterocycle is each optionally substituted with one or more —C₁-C₆ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₆ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₄ alkyl, —C₃-C₆ cycloalkyl, and 4- to 6-membered heterocycle; wherein each alkyl is optionally substituted with one or more —C₁-C₄ alkoxy, —OH, —NH₂, —NHC₁-C₄ alkyl, or N(C₁-C₄ alkyl)₂; wherein said cycloalkyl or heterocycle is each optionally substituted with one or more —C₁-C₄ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₄ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle; wherein each alkyl is optionally substituted with one or more —C₁-C₂ alkoxy, —OH, —NH₂, —NHC₁-C₂ alkyl, or N(C₁-C₂ alkyl)₂; wherein said cycloalkyl or heterocycle is each optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle comprises one or more atoms selected from O and N; wherein each alkyl is optionally substituted with one or more —C₁-C₂ alkoxy, —OH, —NH₂, —NHC₁-C₂ alkyl, or N(C₁-C₂ alkyl)₂; wherein said heterocycle is optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle comprises one or two atoms selected from O and N; wherein each alkyl is optionally substituted with one or more —C₁-C₂ alkoxy, —OH, —NH₂, —NHC₁-C₂ alkyl, or N(C₁-C₂ alkyl)₂; wherein said heterocycle is optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl.

In some embodiments, Y³ is NR⁸⁷ and R⁸⁷ is independently selected from —H, —C₁-C₄ alkyl, —C(O)C₁-C₄ alkyl, —S(O)₂NH₂, —S(O)₂C₁-C₂ alkyl, —C₃-C₆ cycloalkyl, and 4- to 6-membered heterocycle, wherein said heterocycle is selected from piperidine, piperazine, morpholine, pyrrolidine, tetrahydrofuran, azetidine, and oxetane; wherein each alkyl is optionally substituted with one or more —C₁-C₂ alkoxy, —OH, —NH₂, —NHC₁-C₂ alkyl, or N(C₁-C₂ alkyl)₂; wherein said heterocycle is optionally substituted with one or more —C₁-C₂ alkyl; Y¹ is independently, at each occurrence, selected from CR⁸¹R⁸², C(O), and O, preferably Y¹ is independently, at each occurrence, selected from CR⁸¹R⁸² and C(O); Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl.

In some embodiments, Y³ is NR⁸⁷ and R⁸⁷ is independently selected from —H, —C₁-C₄ alkyl, and 4- to 6-membered heterocycle, wherein said heterocycle is selected from piperidine, piperazine, morpholine, tetrahydropyran, pyrrolidine, tetrahydrofuran, azetidine, and oxetane; wherein each alkyl is optionally substituted with one or more —C₁-C₂ alkoxy, —OH, —NH₂, —NHC₁-C₂ alkyl, or N(C₁-C₂ alkyl)₂; wherein said heterocycle is optionally substituted with one or more —C₁-C₂ alkyl; Y¹ is independently, at each occurrence, selected from CR⁸¹R⁸², C(O), and O, preferably Y¹ is CR⁸¹R⁸²; Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl; m is 2 and n is 2.

In some embodiments, Y³ is NR⁸⁷ and R⁸⁷ is independently selected from —H, —C₁-C₂ alkyl, and 4- to 6-membered heterocycle, wherein said heterocycle is selected from piperidine, piperazine, morpholine, tetrahydropyran, pyrrolidine, tetrahydrofuran, azetidine, and oxetane; wherein said heterocycle is optionally substituted with one or more —C₁-C₂ alkyl; Y¹ is independently, at each occurrence, selected from CR⁸¹R⁸², C(O), and O, preferably Y¹ is CR⁸¹R⁸²; Y² is independently, at each occurrence, selected from CR^{81A}R^{82A} and O, preferably Y² is independently at each occurrence CR^{81A}R^{82A}; and R⁸¹, R⁸², R^{81A} and R^{82A} are independently, at each occurrence, selected from —H and —C₁-C₂ alkyl; m is 2 and n is 2.

In some embodiments, R⁸ comprises the Formula (B):

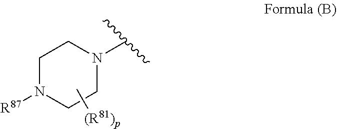

wherein:
R⁸¹ is independently, at each occurrence, selected from —H and —C₁-C₂ alkyl;
R⁸⁷ is independently selected from —H, —C₁-C₂ alkyl, and 4- to 6-membered heterocycle, preferably wherein said heterocycle comprises one heteroatom selected from O and N; and
p is 0, 1 or 2.

In some embodiments, R⁸ comprises the Formula (B), wherein:
R⁸¹ is independently, at each occurrence, selected from —H and methyl;
R⁸⁷ is independently selected from —H, methyl, and 4- to 6-membered heterocycle; and
p is 0, 1 or 2, preferably p is 0 or 2.

In some embodiments, R⁸ comprises the Formula (B), wherein:
R⁸¹ is independently, at each occurrence, selected from —H and methyl;
R⁸⁷ is independently selected from —H, methyl, oxetane, preferably 3-oxetane, and tetrahydropyran, preferably 4-tetrahydropyran; and
p is 0, 1 or 2, preferably p is 0 or 2.

In some embodiments, R⁸ comprises the Formula (B), wherein: R⁸¹ is independently, at each occurrence, selected from —H and methyl;
R⁸⁷ is independently selected from —H, methyl, oxetane, preferably 3-oxetane, and tetrahydropyran, preferably 4-tetrahydropyran; and
p is 0, 1 or 2, preferably p is 0 or 2;
preferably wherein when 2 R⁸¹ are present, they are attached at different carbon atoms.

In some embodiments, R⁸ comprises the Formula (B-1):

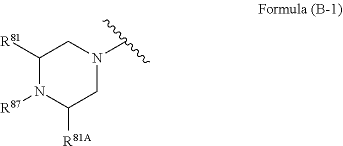

wherein:
R⁸¹ is independently, at each occurrence, selected from —H and —C₁-C₂ alkyl;
R^{81A} is independently, at each occurrence, selected from —H and —C₁-C₂ alkyl;
R⁸⁷ is independently selected from —H, —C₁-C₂ alkyl, and 4- to 6-membered heterocycle, preferably wherein said heterocycle comprises one heteroatom selected from O and N.

In some embodiments, R⁸ comprises the Formula (B-1), wherein:
R⁸¹ is independently, at each occurrence, selected from —H and methyl;
R^{81A} is independently, at each occurrence, selected from —H and methyl;
R¹⁷ is independently selected from —H, methyl, and 4- to 6-membered heterocycle, preferably wherein said heterocycle comprises one heteroatom selected from O and N.

In some embodiments, R⁸ comprises the Formula (B-1), wherein:
R⁸¹ is independently, at each occurrence, selected from —H and methyl;
R^{81A} is independently, at each occurrence, selected from —H and methyl;
R⁸⁷ is independently selected from —H, methyl, oxetane, preferably 3-oxetane, and tetrahydropyran, preferably 4-tetrahydropyran.

In some embodiments, RY comprises the Formula (B-1), wherein:
R⁸¹ and R^{81A} are each together selected from —H and methyl;
R¹⁷ is independently selected from —H, methyl, oxetane, preferably 3-oxetane, and tetrahydropyran, preferably 4-tetrahydropyran.

In some embodiments, RY comprises the Formula (C):

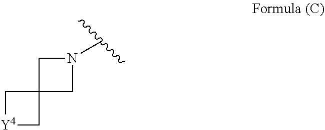

wherein:
Y⁴ is selected from —CR⁹¹R⁹²—, —NR⁹³—, and O;
R⁹¹ and R⁹² are each independently selected from —H, —C₁-C₆ alkyl, —OH, and —COOH; and
R⁹³ is selected from —H and —C₁-C₆ alkyl.

In some embodiments, RY comprises the formula (C), wherein Y⁴ is selected from —CR⁹¹R⁹²—, —NR⁹³—, and O;
R⁹¹ and R⁹² are each independently selected from —H, —C₁-C₆ alkyl, and —OH; and
R⁹³ is selected from —H and —C₁-C₆ alkyl.

In some embodiments, R⁸ comprises the Formula (C), wherein Y⁴ is selected from —CR⁹¹R⁹²— and —NR⁹³—.

In some embodiments, R⁸ comprises Formula (C), wherein Y⁴ is selected from —CR⁹¹R⁹²— and —NR⁹³—;
R⁹¹ and R⁹² are each independently selected from —H, —C₁-C₄ alkyl, —OH and —COOH; and
R⁹³ is selected from —H and —C₁-C₄ alkyl.

In some embodiments, R⁸ comprises Formula (C), wherein Y⁴ is selected from —CR⁹¹R⁹²— and —NR⁹³—;
R⁹¹ and R⁹² are each independently selected from —H, —C₁-C₄ alkyl, and —OH; and
R⁹³ is selected from —H and —C₁-C₄ alkyl.

In some embodiments, R⁸ comprises I Formula (C), wherein Y⁴ is selected from —CR⁹¹R⁹²— and —NR⁹³—;
R⁹¹ and R⁹² are each independently selected from —H, —C₁-C₂ alkyl, —OH and —COOH; and
R⁹³ is selected from —H and —C₁-C₂ alkyl.

In some embodiments, R⁸ comprises I Formula (C), wherein Y⁴ is selected from —CR⁹¹R⁹²— and —NR⁹³—;
R⁹¹ and R⁹² are each independently selected from —H, —C₁-C₂ alkyl, and —OH; and
R⁹³ is selected from —H and —C₁-C₂ alkyl.

In some embodiments, R⁸ comprises the Formula (C), wherein Y⁴ is selected from —CR⁹¹R⁹²— and —NR⁹³—;
R⁹¹ and R⁹² are each independently selected from —H, —CH₃, —OH and —COOH; and
R⁹³ is selected from —H and —CH₃.

In some embodiments, R⁸ comprises the Formula (C), wherein Y⁴ is selected from —CR⁹¹R⁹²— and —NR⁹³—;
R⁹¹ and R⁹² are each independently selected from —H, —CH₃, and —OH; and
R⁹³ is selected from —H and —CH₃.

In some embodiments, R⁸ comprises the Formula (C), wherein Y⁴ is —CR⁹¹R⁹²—; and
R⁹¹ and R⁹² are each independently selected from —H, —CH₃, —OH and —COOH.

In some embodiments, R⁸ comprises the Formula (C), wherein Y⁴ is —CR⁹¹R⁹²—; and
R⁹¹ and R⁹² are each independently selected from —H, —CH₃, and —OH.

In some embodiments, R⁸ comprises the Formula (C), wherein Y⁴ is —NR⁹³—; and
R⁹³ is selected from —H and —CH₃.

In some embodiments, the compound of Formula (I) is selected from:
3-(2,4-dimethylbenzenesulfonyl)-7-hydroxy-8-(4-methylpiperazin-1-yl)-4H,5H-[1,2,3]triazolo[1,5-a]quinazolin-5-one (Compound 1);
3-(2,4-dimethylbenzenesulfonyl)-7-hydroxy-8-{6-methyl-2,6-diazaspiro[3.3]heptan-2-yl}-4H,5H-[1,2,3]triazolo[1,5-a]quinazolin-5-one (Compound 2);
3-(2,4-dimethylbenzenesulfonyl)-8-{8-methyl-2,8-diazaspiro[4.5]decan-2-yl}-1H,5H-[1,2,3]triazolo[1,5-a]quinazolin-5-one (Compound 3);
3-(2,4-dimethylbenzenesulfonyl)-8-{2-methyl-2,9-diazaspiro[5.5]undecan-9-yl}-1H,5H-[1,2,3]triazolo[1,5-a]quinazolin-5-one (Compound 4);
3-(2,4-dimethylbenzenesulfonyl)-8-{9-methyl-2,9-diazaspiro[5.5]undecan-2-yl}-1H,5H-[1,2,3]triazolo[1,5-a]quinazolin-5-one (Compound 5);
3-(2,4-dimethylbenzenesulfonyl)-8-{9-methyl-3,9-diazaspiro[5.5]undecan-3-yl}-1H,5H-[1,2,3]triazolo[1,5-a]quinazolin-5-one (Compound 6);
3-(2,4-dimethylbenzenesulfonyl)-8-{6-oxa-2,9-diazaspiro[4.5]decan-2-yl}-4H,5H-[1,2,3]triazolo[1,5-a]quinazolin-5-one (Compound 7);
3-(2,4-dimethylbenzenesulfonyl)-8-{9-methyl-6-oxa-2,9-diazaspiro[4.5]decan-2-yl}-4H,5H-[1,2,3]triazolo[1,5-a]quinazolin-5-one (Compound 8);
3-(2,4-dimethylbenzenesulfonyl)-8-{5-oxa-2,8-diazaspiro[3.5]nonan-8-yl}-4H,5H-[1,2,3]triazolo[1,5-a]quinazolin-5-one (Compound 9);
3-(2,4-dimethylbenzenesulfonyl)-8-{2-methyl-5-oxa-2,8-diazaspiro[3.5]nonan-8-yl}-4H,5H-[1,2,3]triazolo[1,5-a]quinazolin-5-one (Compound 10);
3-(2,4-dimethylbenzenesulfonyl)-8-{6-methyl-1,6-diazaspiro[3.3]heptan-1-yl}-4H,5H-[1,2,3]triazolo[1,5-a]quinazolin-5-one (Compound 11);
3-(2,4-dimethylbenzenesulfonyl)-8-{1-methyl-1,6-diazaspiro[3.3]heptan-6-yl}-4H,5H-[1,2,3]triazolo[1,5-a]quinazolin-5-one (Compound 12);
3-(2,4-dimethylbenzenesulfonyl)-8-{2-oxa-6-azaspiro[3.3]heptan-6-yl}-4H,5H-[1,2,3]triazolo[1,5-a]quinazolin-5-one (Compound 13);
3-(2,4-dimethylbenzenesulfonyl)-8-{6-methyl-2,6-diazaspiro[3.3]heptan-2-yl}-4H,5H-[1,2,3]triazolo[1,5-a]quinazolin-5-one (Compound 14);
3-(2,4-dimethylbenzenesulfonyl)-8-{1-oxa-4,9-diazaspiro[5.5]undecan-9-yl}-1H,5H-[1,2,3]triazolo[1,5-a]quinazolin-5-one (Compound 15);
3-(2,4-dimethylbenzenesulfonyl)-8-{4-methyl-1-oxa-4,9-diazaspiro[5.5]undecan-9-yl}-1H,5H-[1,2,3]triazolo[1,5-a]quinazolin-5-one (Compound 16);
3-(2,4-dimethylbenzenesulfonyl)-8-{1-oxa-4,9-diazaspiro[5.5]undecan-4-yl}-1H,5H-[1,2,3]triazolo[1,5-a]quinazolin-5-one (Compound 17);
3-(2,4-dimethylbenzenesulfonyl)-8-{9-methyl-1-oxa-4,9-diazaspiro[5.5]undecan-4-yl}-1H,5H-[1,2,3]triazolo[1,5-a]quinazolin-5-one (Compound 18);
3-(2,4-dimethylbenzenesulfonyl)-8-[(7R)-7-hydroxy-3,9-diazaspiro[5.5]undecan-3-yl]-1H,5H-[1,2,3]triazolo[1,5-a]quinazolin-5-one (Compound 19);
3-(2,4-dimethylbenzenesulfonyl)-8-{6-hydroxy-2-azaspiro[3.3]heptan-2-yl}-1H,5H-[1,2,3]triazolo[1,5-a]quinazolin-5-one (Compound 20);
3-(2,4-dimethylbenzenesulfonyl)-8-{6-hydroxy-6-methyl-2-azaspiro[3.3]heptan-2-yl}-1H,5H-[1,2,3]triazolo[1,5-a]quinazolin-5-one (Compound 21);
3-(2,4-dimethylbenzenesulfonyl)-8-(4-methylpiperazin-1-yl)-4H,5H-[1,2,3]triazolo[1,5-a]quinazolin-5-one (Compound 22);
3-(2,4-dimethylbenzenesulfonyl)-8-(piperazin-1-yl)-4H,5H-[1,2,3]triazolo[1,5-a]quinazolin-5-one (Compound 23);
3-(2,4-dimethylbenzenesulfonyl)-8-[(3R,5S)-3,5-dimethylpiperazin-1-yl]-4H,5H-[1,2,3]triazolo[1,5-a]quinazolin-5-one (Compound 24);
rac-3-(2,4-dimethylbenzenesulfonyl)-8-[(3R,5S)-3,4,5-trimethylpiperazin-1-yl]-4H,5H-[1,2,3]triazolo[1,5-a]quinazolin-5-one (Compound 25);
3-(2,4-dimethylbenzenesulfonyl)-8-(3,5-dimethylpiperazin-1-yl)-4H,5H-[1,2,3]triazolo[1,5-a]quinazolin-5-one (Compound 26);
3-(2,4-dimethylbenzenesulfonyl)-8-(3,4,5-trimethylpiperazin-1-yl)-4H,5H-[1,2,3]triazolo[1,5-a]quinazolin-5-one (Compound 27);
rac-3-(2,4-dimethylbenzenesulfonyl)-8-[(2R,5S)-2,5-dimethylpiperazin-1-yl]-4H,5H-[1,2,3]triazolo[1,5-a]quinazolin-5-one (Compound 28);
3-(2,4-dimethylbenzenesulfonyl)-8-(3,3,4-trimethylpiperazin-1-yl)-4H,5H-[1,2,3]triazolo[1,5-a]quinazolin-5-one (Compound 29);
3-(2,4-dimethylbenzenesulfonyl)-8-{octahydropyrazino[2,1-c][1,4]oxazin-8-yl}-4H,5H-[1,2,3]triazolo[1,5-a]quinazolin-5-one (Compound 30);
3-(2,4-dimethylbenzenesulfonyl)-8-[4-(methylamino)piperidin-1-yl]-4H,5H-[1,2,3]triazolo[1,5-a]quinazolin-5-one (Compound 31);
8-[4-(dimethylamino)piperidin-1-yl]-3-(2,4-dimethylbenzenesulfonyl)-4H,5H-[1,2,3]triazolo[1,5-a]quinazolin-5-one (Compound 32);
8-(4-aminopiperidin-1-yl)-3-(2,4-dimethylbenzenesulfonyl)-4H,5H-[1,2,3]triazolo[1,5-a]quinazolin-5-one (Compound 33);
3-(2,4-dimethylbenzenesulfonyl)-8-[3-(methylamino)pyrrolidin-1-yl]-4H,5H-[1,2,3]triazolo[1,5-a]quinazolin-5-one (Compound 34);
8-[(3R)-3-aminopyrrolidin-1-yl]-3-(2,4-dimethylbenzenesulfonyl)-4H,5H-[1,2,3]triazolo[1,5-a]quinazolin-5-one (Compound 35);
3-(2,4-dimethylbenzenesulfonyl)-8-{octahydropyrrolo[1,2-a]pyrazin-2-yl}-4H,5H-[1,2,3]triazolo[1,5-a]quinazolin-5-one (Compound 36);
8-[(8aS)-octahydropyrrolo[1,2-a]pyrazin-2-yl]-3-(2,4-dimethylbenzenesulfonyl)-4H,5H-[1,2,3]triazolo[1,5-a]quinazolin-5-one (Compound 37);
8-[(8aR)-octahydropyrrolo[1,2-a]pyrazin-2-yl]-3-(2,4-dimethylbenzenesulfonyl)-4H,5H-[1,2,3]triazolo[1,5-a]quinazolin-5-one (Compound 38);
3-(2,4-dimethylbenzenesulfonyl)-8-{1-methyl-octahydro-1H-pyrrolo[3,2-c]pyridin-5-yl}-4H,5H-[1,2,3]triazolo[1,5-a]quinazolin-5-one (Compound 39);
8-[(3aR,6aS)-octahydropyrrolo[3,4-c]pyrrol-2-yl]-3-(2,4-dimethylbenzenesulfonyl)-4H,5H-[1,2,3]triazolo[1,5-a]quinazolin-5-one (Compound 40);
8-(4-tert-butylpiperazin-1-yl)-3-(2,4-dimethylbenzenesulfonyl)-4H,5H-[1,2,3]triazolo[1,5-a]quinazolin-5-one (Compound 41);
3-(2,4-dimethylbenzenesulfonyl)-8-[4-(2-methoxyethyl)piperazin-1-yl]-4H,5H-[1,2,3]triazolo[1,5-a]quinazolin-5-one (Compound 42);
3-(2,4-dimethylbenzenesulfonyl)-8-[4-(2-hydroxyethyl)piperazin-1-yl]-4H,5H-[1,2,3]triazolo[1,5-a]quinazolin-5-one (Compound 43);
3-(2,4-dimethylbenzenesulfonyl)-8-[4-(1-hydroxypropan-2-yl)piperazin-1-yl]-4H,5H-[1,2,3]triazolo[1,5-a]quinazolin-5-one (Compound 44);
3-(2,4-dimethylbenzenesulfonyl)-8-{4-[2-(2-hydroxyethoxy)ethyl]piperazin-1-yl}-4H,5H-[1,2,3]triazolo[1,5-a]quinazolin-5-one (Compound 45);
3-(2,4-dimethylbenzenesulfonyl)-8-[4-(2-hydroxyethyl)-1,4-diazepan-1-yl]-4H,5H-[1,2,3]triazolo[1,5-a]quinazolin-5-one (Compound 46);
8-(1,4-diazepan-1-yl)-3-(2,4-dimethylbenzenesulfonyl)-4H,5H-[1,2,3]triazolo[1,5-a]quinazolin-5-one (Compound 47);
3-(2,4-dimethylbenzenesulfonyl)-8-(1,4-oxazepan-4-yl)-4H,5H-[1,2,3]triazolo[1,5-a]quinazolin-5-one (Compound 48);
3-(2,4-dimethylbenzenesulfonyl)-8-(4-methyl-1,4-diazepan-1-yl)-4H,5H-[1,2,3]triazolo[1,5-a]quinazolin-5-one (Compound 49);
8-{[(1R,3R,5S)-8-azabicyclo[3.2.1]octan-3-yl]amino}-3-(2,4-dimethylbenzenesulfonyl)-1H,5H-[1,2,3]triazolo[1,5-a]quinazolin-5-one (Compound 50);
3-(2,4-dimethylbenzenesulfonyl)-8-[4-(2,2,2-trifluoroethyl)piperazin-1-yl]-4H,5H-[1,2,3]triazolo[1,5-a]quinazolin-5-one (Compound 51);
3-(2,4-dimethylbenzenesulfonyl)-8-[4-(2-methylpropanoyl)piperazin-1-yl]-4H,5H-[1,2,3]triazolo[1,5-a]quinazolin-5-one (Compound 52);
4-[3-(2,4-dimethylbenzenesulfonyl)-5-oxo-4H,5H-[1,2,3]triazolo[1,5-a]quinazolin-8-yl]-1-methylpiperazin-2-one (Compound 53);
3-(2,4-dimethylbenzenesulfonyl)-8-{5H,6H,7H,8H-imidazo[1,2-a]pyrazin-7-yl}-4H,5H-[1,2,3]triazolo[1,5-a]quinazolin-5-one (Compound 54);
3-(2,4-dimethylbenzenesulfonyl)-8-[4-(oxetan-3-yl)piperazin-1-yl]-4H,5H-[1,2,3]triazolo[1,5-a]quinazolin-5-one (Compound 55);
3-(2,4-dimethylbenzenesulfonyl)-8-[4-(oxan-4-yl)piperazin-1-yl]-1H,5H-[1,2,3]triazolo[1,5-a]quinazolin-5-one (Compound 56);
8-[4-(2-aminoethyl)piperazin-1-yl]-3-(2,4-dimethylbenzenesulfonyl)-4H,5H-[1,2,3]triazolo[1,5-a]quinazolin-5-one (Compound 57);
8-{4-[3-(diethylamino)propanoyl]piperazin-1-yl}-3-(2,4-dimethylbenzenesulfonyl)-4H,5H-[1,2,3]triazolo[1,5-a]quinazolin-5-one (Compound 58);
3-(2,4-dimethylbenzenesulfonyl)-8-[4-(1-methylpiperidin-4-yl)piperazin-1-yl]-1H,5H-[1,2,3]triazolo[1,5-a]quinazolin-5-one (Compound 59);
8-(4-cyclopropylpiperazin-1-yl)-3-(2,4-dimethylbenzenesulfonyl)-4H,5H-[1,2,3]triazolo[1,5-a]quinazolin-5-one (Compound 60);
3-(2,4-dimethylbenzenesulfonyl)-8-(4-methanesulfonylpiperazin-1-yl)-4H,5H-[1,2,3]triazolo[1,5-a]quinazolin-5-one (Compound 61);
4-[3-(2,4-dimethylbenzenesulfonyl)-5-oxo-4H,5H-[1,2,3]triazolo[1,5-a]quinazolin-8-yl]piperazine-1-sulfonamide (Compound 62);
2-[3-(2,4-Dimethyl-benzenesulfonyl)-5-oxo-4,5-dihydro-[1,2,3]triazolo[1,5-a]quinazolin-8-yl]-2-aza-spiro[3.3]heptane-6-carboxylic acid (Compound 63); and
1-[3-(2,4-Dimethyl-benzenesulfonyl)-5-oxo-4,5-dihydro-[1,2,3]triazolo[1,5-a]quinazolin-8-yl]-piperidine-4-carboxylic acid (Compound 64);
   or a pharmaceutically acceptable salt thereof.

In some embodiments, the compound of Formula (I) is selected from:
3-(2,4-dimethylbenzenesulfonyl)-7-hydroxy-8-(4-methylpiperazin-1-yl)-4H,5H-[1,2,3]triazolo[1,5-a]quinazolin-5-one (Compound 1);
3-(2,4-dimethylbenzenesulfonyl)-7-hydroxy-8-{6-methyl-2,6-diazaspiro[3.3]heptan-2-yl}-4H,5H-[1,2,3]triazolo[1,5-a]quinazolin-5-one (Compound 2);
3-(2,4-dimethylbenzenesulfonyl)-8-{8-methyl-2,8-diazaspiro[4.5]decan-2-yl}-1H,5H-[1,2,3]triazolo[1,5-a]quinazolin-5-one (Compound 3);
3-(2,4-dimethylbenzenesulfonyl)-8-{6-oxa-2,9-diazaspiro[4.5]decan-2-yl}-4H,5H-[1,2,3]triazolo[1,5-a]quinazolin-5-one (Compound 7);
3-(2,4-dimethylbenzenesulfonyl)-8-{9-methyl-6-oxa-2,9-diazaspiro[4.5]decan-2-yl}-4H,5H-[1,2,3]triazolo[1,5-a]quinazolin-5-one (Compound 8);
3-(2,4-dimethylbenzenesulfonyl)-8-{1-methyl-,6-diazaspiro[3.3]heptan-6-yl}-4H,5H-[1,2,3]triazolo[1,5-a]quinazolin-5-one (Compound 12);
3-(2,4-dimethylbenzenesulfonyl)-8-{6-methyl-2,6-diazaspiro[3.3]heptan-2-yl}-4H,5H-[1,2,3]triazolo[1,5-a]quinazolin-5-one (Compound 14);
3-(2,4-dimethylbenzenesulfonyl)-8-{4-methyl-1-oxa-4,9-diazaspiro[5.5]undecan-9-yl}-1H,5H-[1,2,3]triazolo[1,5-a]quinazolin-5-one (Compound 16);
3-(2,4-dimethylbenzenesulfonyl)-8-{6-hydroxy-6-methyl-2-azaspiro[3.3]heptan-2-yl}-1H,5H-[1,2,3]triazolo[1,5-a]quinazolin-5-one (Compound 21);
3-(2,4-dimethylbenzenesulfonyl)-8-(4-methylpiperazin-1-yl)-4H,5H-[1,2,3]triazolo[1,5-a]quinazolin-5-one (Compound 22);
3-(2,4-dimethylbenzenesulfonyl)-8-(piperazin-1-yl)-4H,5H-[1,2,3]triazolo[1,5-a]quinazolin-5-one (Compound 23);
3-(2,4-dimethylbenzenesulfonyl)-8-[(3R,5S)-3,5-dimethylpiperazin-1-yl]-4H,5H-[1,2,3]triazolo[1,5-a]quinazolin-5-one (Compound 24);
rac-3-(2,4-dimethylbenzenesulfonyl)-8-[(3R,5S)-3,4,5-trimethylpiperazin-1-yl]-4H,5H-[1,2,3]triazolo[1,5-a]quinazolin-5-one (Compound 25);
rac-3-(2,4-dimethylbenzenesulfonyl)-8-[(2R,5S)-2,5-dimethylpiperazin-1-yl]-4H,5H-[1,2,3]triazolo[1,5-a]quinazolin-5-one (Compound 28);
3-(2,4-dimethylbenzenesulfonyl)-8-[4-(methylamino)piperidin-1-yl]-4H,5H-[1,2,3]triazolo[1,5-a]quinazolin-5-one (Compound 31);
3-(2,4-dimethylbenzenesulfonyl)-8-{octahydropyrrolo[1,2-a]pyrazin-2-yl}-4H,5H-[1,2,3]triazolo[1,5-a]quinazolin-5-one (Compound 36);
3-(2,4-dimethylbenzenesulfonyl)-8-{1-methyl-octahydro-1H-pyrrolo[3,2-c]pyridin-5-yl}-4H,5H-[1,2,3]triazolo[1,5-a]quinazolin-5-one (Compound 39);
3-(2,4-dimethylbenzenesulfonyl)-8-{4-[2-(2-hydroxyethoxy)ethyl]piperazin-1-yl}-4H,5H-[1,2,3]triazolo[1,5-a]quinazolin-5-one (Compound 45);
3-(2,4-dimethylbenzenesulfonyl)-8-[4-(2-hydroxyethyl)-1,4-diazepan-1-yl]-4H,5H-[1,2,3]triazolo[1,5-a]quinazolin-5-one (Compound 46);
8-(1,4-diazepan-1-yl)-3-(2,4-dimethylbenzenesulfonyl)-4H,5H-[1,2,3]triazolo[1,5-a]quinazolin-5-one (Compound 47);
3-(2,4-dimethylbenzenesulfonyl)-8-(1,4-oxazepan-4-yl)-4H,5H-[1,2,3]triazolo[1,5-a]quinazolin-5-one (Compound 48);
3-(2,4-dimethylbenzenesulfonyl)-8-[4-(2-methylpropanoyl)piperazin-1-yl]-4H,5H-[1,2,3]triazolo[1,5-a]quinazolin-5-one (Compound 52);
4-[3-(2,4-dimethylbenzenesulfonyl)-5-oxo-4H,5H-[1,2,3]triazolo[1,5-a]quinazolin-8-yl]-1-methylpiperazin-2-one (Compound 53);
3-(2,4-dimethylbenzenesulfonyl)-8-[4-(oxetan-3-yl)piperazin-1-yl]-4H,5H-[1,2,3]triazolo[1,5-a]quinazolin-5-one (Compound 55);
3-(2,4-dimethylbenzenesulfonyl)-8-[4-(oxan-4-yl)piperazin-1-yl]-1H,5H-[1,2,3]triazolo[1,5-a]quinazolin-5-one (Compound 56);
8-(4-cyclopropylpiperazin-1-yl)-3-(2,4-dimethylbenzenesulfonyl)-4H,5H-[1,2,3]triazolo[1,5-a]quinazolin-5-one (Compound 60);
3-(2,4-dimethylbenzenesulfonyl)-8-(4-methanesulfonylpiperazin-1-yl)-4H,5H-[1,2,3]triazolo[1,5-a]quinazolin-5-one (Compound 61);
4-[3-(2,4-dimethylbenzenesulfonyl)-5-oxo-4H,5H-[1,2,3]triazolo[1,5-a]quinazolin-8-yl]piperazine-1-sulfonamide (Compound 62);
2-[3-(2,4-Dimethyl-benzenesulfonyl)-5-oxo-4,5-dihydro-[1,2,3]triazolo[1,5-a]quinazolin-8-yl]-2-aza-spiro[3.3]heptane-6-carboxylic acid (Compound 63); and
1-[3-(2,4-Dimethyl-benzenesulfonyl)-5-oxo-4,5-dihydro-[1,2,3]triazolo[1,5-a]quinazolin-8-yl]-piperidine-4-carboxylic acid (Compound 64); or a pharmaceutically acceptable salt thereof.

In some embodiments, the compound of Formula (I) is 3-(2,4-dimethylbenzenesulfonyl)-7-hydroxy-8-(4-methylpiperazin-1-yl)-4H,5H-[1,2,3]triazolo[1,5-a]quinazolin-5-one (Compound 1); or a pharmaceutically acceptable salt thereof.

In some embodiments, the compound of Formula (I) is 3-(2,4-dimethylbenzenesulfonyl)-7-hydroxy-8-{6-methyl-2,6-diazaspiro[3.3]heptan-2-yl}-4H,5H-[1,2,3]triazolo[1,5-a]quinazolin-5-one (Compound 2); or a pharmaceutically acceptable salt thereof.

In some embodiments, the compound of Formula (I) is 3-(2,4-dimethylbenzenesulfonyl)-8-{8-methyl-2,8-diazaspiro[4.5]decan-2-yl}-1H,5H-[1,2,3]triazolo[1,5-a]quinazolin-5-one (Compound 3); or a pharmaceutically acceptable salt thereof.

In some embodiments, the compound of Formula (I) is 3-(2,4-dimethylbenzenesulfonyl)-8-{2-methyl-2,9-diazaspiro[5.5]undecan-9-yl}-1H,5H-[1,2,3]triazolo[1,5-a]quinazolin-5-one (Compound 4); or a pharmaceutically acceptable salt thereof.

In some embodiments, the compound of Formula (I) is 3-(2,4-dimethylbenzenesulfonyl)-8-{9-methyl-2,9-diazaspiro[5.5]undecan-2-yl}-1H,5H-[1,2,3]triazolo[1,5-a]quinazolin-5-one (Compound 5); or a pharmaceutically acceptable salt thereof.

In some embodiments, the compound of Formula (I) is 3-(2,4-dimethylbenzenesulfonyl)-8-{9-methyl-3,9-diazaspiro[5.5]undecan-3-yl}-1H,5H-[1,2,3]triazolo[1,5-a]quinazolin-5-one (Compound 6); or a pharmaceutically acceptable salt thereof.

In some embodiments, the compound of Formula (I) is 3-(2,4-dimethylbenzenesulfonyl)-8-{6-oxa-2,9-diazaspiro[4.5]decan-2-yl}-4H,5H-[1,2,3]triazolo[1,5-a]quinazolin-5-one (Compound 7); or a pharmaceutically acceptable salt thereof.

In some embodiments, the compound of Formula (I) is 3-(2,4-dimethylbenzenesulfonyl)-8-{9-methyl-6-oxa-2,9-diazaspiro[4.5]decan-2-yl}-4H,5H-[1,2,3]triazolo[1,5-a]quinazolin-5-one (Compound 8); or a pharmaceutically acceptable salt thereof.

In some embodiments, the compound of Formula (I) is 3-(2,4-dimethylbenzenesulfonyl)-8-{5-oxa-2,8-diazaspiro[3.5]nonan-8-yl}-4H,5H-[1,2,3]triazolo[1,5-a]quinazolin-5-one (Compound 9); or a pharmaceutically acceptable salt thereof.

In some embodiments, the compound of Formula (I) is 3-(2,4-dimethylbenzenesulfonyl)-8-{2-methyl-5-oxa-2,8-diazaspiro[3.5]nonan-8-yl}-4H,5H-[1,2,3]triazolo[1,5-a]quinazolin-5-one (Compound 10); or a pharmaceutically acceptable salt thereof.

In some embodiments, the compound of Formula (I) is 3-(2,4-dimethylbenzenesulfonyl)-8-{6-methyl-1,6-diazaspiro[3.3]heptan-1-yl}-4H,5H-[1,2,3]triazolo[1,5-a]quinazolin-5-one (Compound 11); or a pharmaceutically acceptable salt thereof.

In some embodiments, the compound of Formula (I) is 3-(2,4-dimethylbenzenesulfonyl)-8-{1-methyl-1,6-diazaspiro[3.3]heptan-6-yl}-4H,5H-[1,2,3]triazolo[1,5-a]quinazolin-5-one (Compound 12); or a pharmaceutically acceptable salt thereof.

In some embodiments, the compound of Formula (I) is 3-(2,4-dimethylbenzenesulfonyl)-8-{2-oxa-6-azaspiro[3.3]heptan-6-yl}-4H,5H-[1,2,3]triazolo[1,5-a]quinazolin-5-one (Compound 13); or a pharmaceutically acceptable salt thereof.

In some embodiments, the compound of Formula (I) is 3-(2,4-dimethylbenzenesulfonyl)-8-{6-methyl-2,6-diazaspiro[3.3]heptan-2-yl}-4H,5H-[1,2,3]triazolo[1,5-a]quinazolin-5-one (Compound 14); or a pharmaceutically acceptable salt thereof.

In some embodiments, the compound of Formula (I) is 3-(2,4-dimethylbenzenesulfonyl)-8-{1-oxa-4,9-diazaspiro[5.5]undecan-9-yl}-1H,5H-[1,2,3]triazolo[1,5-a]quinazolin-5-one (Compound 15); or a pharmaceutically acceptable salt thereof.

In some embodiments, the compound of Formula (I) is 3-(2,4-dimethylbenzenesulfonyl)-8-{4-methyl-1-oxa-4,9-diazaspiro[5.5]undecan-9-yl}-1H,5H-[1,2,3]triazolo[1,5-a]quinazolin-5-one (Compound 16); or a pharmaceutically acceptable salt thereof.

In some embodiments, the compound of Formula (I) is 3-(2,4-dimethylbenzenesulfonyl)-8-{1-oxa-4,9-diazaspiro[5.5]undecan-4-yl}-1H,5H-[1,2,3]triazolo[1,5-a]quinazolin-5-one (Compound 17); or a pharmaceutically acceptable salt thereof.

In some embodiments, the compound of Formula (I) is 3-(2,4-dimethylbenzenesulfonyl)-8-{9-methyl-1-oxa-4,9-diazaspiro[5.5]undecan-4-yl}-1H,5H-[1,2,3]triazolo[1,5-a]quinazolin-5-one (Compound 18); or a pharmaceutically acceptable salt thereof.

In some embodiments, the compound of Formula (I) is 3-(2,4-dimethylbenzenesulfonyl)-8-[(7R)-7-hydroxy-3,9-diazaspiro[5.5]undecan-3-yl]-1H,5H-[1,2,3]triazolo[1,5-a]quinazolin-5-one (Compound 19); or a pharmaceutically acceptable salt thereof.

In some embodiments, the compound of Formula (I) is 3-(2,4-dimethylbenzenesulfonyl)-8-{6-hydroxy-2-azaspiro[3.3]heptan-2-yl}-1H,5H-[1,2,3]triazolo[1,5-a]quinazolin-5-one (Compound 20); or a pharmaceutically acceptable salt thereof.

In some embodiments, the compound of Formula (I) is 3-(2,4-dimethylbenzenesulfonyl)-8-{6-hydroxy-6-methyl-2-azaspiro[3.3]heptan-2-yl}-1H,5H-[1,2,3]triazolo[1,5-a]quinazolin-5-one (Compound 21); or a pharmaceutically acceptable salt thereof.

In some embodiments, the compound of Formula (I) is 3-(2,4-dimethylbenzenesulfonyl)-8-(4-methylpiperazin-1-yl)-4H,5H-[1,2,3]triazolo[1,5-a]quinazolin-5-one (Compound 22); or a pharmaceutically acceptable salt thereof.

In some embodiments, the compound of Formula (I) is 3-(2,4-dimethylbenzenesulfonyl)-8-(piperazin-1-yl)-4H,5H-[1,2,3]triazolo[1,5-a]quinazolin-5-one (Compound 23); or a pharmaceutically acceptable salt thereof.

In some embodiments, the compound of Formula (I) is 3-(2,4-dimethylbenzenesulfonyl)-8-[(3R,5S)-3,5-dimethylpiperazin-1-yl]-4H,5H-[1,2,3]triazolo[1,5-a]quinazolin-5-one (Compound 24); or a pharmaceutically acceptable salt thereof.

In some embodiments, the compound of Formula (I) is rac-3-(2,4-dimethylbenzenesulfonyl)-8-[(3R,5S)-3,4,5-trimethylpiperazin-1-yl]-4H,5H-[1,2,3]triazolo[1,5-a]quinazolin-5-one (Compound 25); or a pharmaceutically acceptable salt thereof.

In some embodiments, the compound of Formula (I) is 3-(2,4-dimethylbenzenesulfonyl)-8-(3,5-dimethylpiperazin-1-yl)-4H,5H-[1,2,3]triazolo[1,5-a]quinazolin-5-one (Compound 26); or a pharmaceutically acceptable salt thereof.

In some embodiments, the compound of Formula (I) is 3-(2,4-dimethylbenzenesulfonyl)-8-(3,4,5-trimethylpiperazin-1-yl)-4H,5H-[1,2,3]triazolo[1,5-a]quinazolin-5-one (Compound 27); or a pharmaceutically acceptable salt thereof.

In some embodiments, the compound of Formula (I) is rac-3-(2,4-dimethylbenzenesulfonyl)-8-[(2R,5S)-2,5-dimethylpiperazin-1-yl]-4H,5H-[1,2,3]triazolo[1,5-a]quinazolin-5-one (Compound 28); or a pharmaceutically acceptable salt thereof.

In some embodiments, the compound of Formula (I) is 3-(2,4-dimethylbenzenesulfonyl)-8-(3,3,4-trimethylpiperazin-1-yl)-4H,5H-[1,2,3]triazolo[1,5-a]quinazolin-5-one (Compound 29); or a pharmaceutically acceptable salt thereof.

In some embodiments, the compound of Formula (I) is 3-(2,4-dimethylbenzenesulfonyl)-8-{octahydropyrazino[2,1-c][1,4]oxazin-8-yl}-4H,5H-[1,2,3]triazolo[1,5-a]quinazolin-5-one (Compound 30); or a pharmaceutically acceptable salt thereof.

In some embodiments, the compound of Formula (I) is 3-(2,4-dimethylbenzenesulfonyl)-8-[4-(methylamino)piperidin-1-yl]-4H,5H-[1,2,3]triazolo[1,5-a]quinazolin-5-one (Compound 31); or a pharmaceutically acceptable salt thereof.

In some embodiments, the compound of Formula (I) is 8-[4-(dimethylamino)piperidin-1-yl]-3-(2,4-dimethylbenzenesulfonyl)-4H,5H-[1,2,3]triazolo[1,5-a]quinazolin-5-one (Compound 32); or a pharmaceutically acceptable salt thereof.

In some embodiments, the compound of Formula (I) is 8-(4-aminopiperidin-1-yl)-3-(2,4-dimethylbenzenesulfonyl)-4H,5H-[1,2,3]triazolo[1,5-a]quinazolin-5-one (Compound 33); or a pharmaceutically acceptable salt thereof.

In some embodiments, the compound of Formula (I) is 3-(2,4-dimethylbenzenesulfonyl)-8-[3-(methylamino)pyrrolidin-1-yl]-4H,5H-[1,2,3]triazolo[1,5-a]quinazolin-5-one (Compound 34); or a pharmaceutically acceptable salt thereof.

In some embodiments, the compound of Formula (I) is 8-[(3R)-3-aminopyrrolidin-1-yl]-3-(2,4-dimethylbenzenesulfonyl)-4H,5H-[1,2,3]triazolo[1,5-a]quinazolin-5-one (Compound 35); or a pharmaceutically acceptable salt thereof.

In some embodiments, the compound of Formula (I) is 3-(2,4-dimethylbenzenesulfonyl)-8-{octahydropyrrolo[1,2-a]pyrazin-2-yl}-4H,5H-[1,2,3]triazolo[1,5-a]quinazolin-5-one (Compound 36); or a pharmaceutically acceptable salt thereof.

In some embodiments, the compound of Formula (I) is 8-[(8aS)-octahydropyrrolo[1,2-a]pyrazin-2-yl]-3-(2,4-dimethylbenzenesulfonyl)-4H,5H-[1,2,3]triazolo[1,5-a]quinazolin-5-one (Compound 37); or a pharmaceutically acceptable salt thereof.

In some embodiments, the compound of Formula (I) is 8-[(8aR)-octahydropyrrolo[1,2-a]pyrazin-2-yl]-3-(2,4-dimethylbenzenesulfonyl)-4H,5H-[1,2,3]triazolo[1,5-a]quinazolin-5-one (Compound 38); or a pharmaceutically acceptable salt thereof.

In some embodiments, the compound of Formula (I) is 3-(2,4-dimethylbenzenesulfonyl)-8-{1-methyl-octahydro-1H-pyrrolo[3,2-c]pyridin-5-yl}-4H,5H-[1,2,3]triazolo[1,5-a]quinazolin-5-one (Compound 39); or a pharmaceutically acceptable salt thereof.

In some embodiments, the compound of Formula (I) is 8-[(3aR,6aS)-octahydropyrrolo[3,4-c]pyrrol-2-yl]-3-(2,4-dimethylbenzenesulfonyl)-4H,5H-[1,2,3]triazolo[1,5-a]quinazolin-5-one (Compound 40); or a pharmaceutically acceptable salt thereof.

In some embodiments, the compound of Formula (I) is 8-(4-tert-butylpiperazin-1-yl)-3-(2,4-dimethylbenzenesulfonyl)-4H,5H-[1,2,3]triazolo[1,5-a]quinazolin-5-one (Compound 41); or a pharmaceutically acceptable salt thereof.

In some embodiments, the compound of Formula (I) is 3-(2,4-dimethylbenzenesulfonyl)-8-[4-(2-methoxyethyl)piperazin-1-yl]-4H,5H-[1,2,3]triazolo[1,5-a]quinazolin-5-one (Compound 42); or a pharmaceutically acceptable salt thereof.

In some embodiments, the compound of Formula (I) is 3-(2,4-dimethylbenzenesulfonyl)-8-[4-(2-hydroxyethyl)piperazin-1-yl]-4H,5H-[1,2,3]triazolo[1,5-a]quinazolin-5-one (Compound 43); or a pharmaceutically acceptable salt thereof.

In some embodiments, the compound of Formula (I) is 3-(2,4-dimethylbenzenesulfonyl)-8-[4-(1-hydroxypropan-2-yl)piperazin-1-yl]-4H,5H-[1,2,3]triazolo[1,5-a]quinazolin-5-one (Compound 44); or a pharmaceutically acceptable salt thereof.

In some embodiments, the compound of Formula (I) is 3-(2,4-dimethylbenzenesulfonyl)-8-{4-[2-(2-hydroxyethoxy)ethyl]piperazin-1-yl}-4H,5H-[1,2,3]triazolo[1,5-a]quinazolin-5-one (Compound 45); or a pharmaceutically acceptable salt thereof.

In some embodiments, the compound of Formula (I) is 3-(2,4-dimethylbenzenesulfonyl)-8-[4-(2-hydroxyethyl)-1,4-diazepan-1-yl]-4H,5H-[1,2,3]triazolo[1,5-a]quinazolin-5-one (Compound 46); or a pharmaceutically acceptable salt thereof.

In some embodiments, the compound of Formula (I) is 8-(1,4-diazepan-1-yl)-3-(2,4-dimethylbenzenesulfonyl)-4H,5H-[1,2,3]triazolo[1,5-a]quinazolin-5-one (Compound 47); or a pharmaceutically acceptable salt thereof.

In some embodiments, the compound of Formula (I) is 3-(2,4-dimethylbenzenesulfonyl)-8-(1,4-oxazepan-4-yl)-4H,5H-[1,2,3]triazolo[1,5-a]quinazolin-5-one (Compound 48); or a pharmaceutically acceptable salt thereof.

In some embodiments, the compound of Formula (I) is 3-(2,4-dimethylbenzenesulfonyl)-8-(4-methyl-1,4-diazepan-1-yl)-4H,5H-[1,2,3]triazolo[1,5-a]quinazolin-5-one (Compound 49); or a pharmaceutically acceptable salt thereof.

In some embodiments, the compound of Formula (I) is 8-{[(1R,3R,5S)-8-azabicyclo[3.2.1]octan-3-yl]amino}-3-(2,4-dimethylbenzenesulfonyl)-1H,5H-[1,2,3]triazolo[1,5-a]quinazolin-5-one (Compound 50); or a pharmaceutically acceptable salt thereof.

In some embodiments, the compound of Formula (I) is 3-(2,4-dimethylbenzenesulfonyl)-8-[4-(2,2,2-trifluoroethyl)piperazin-1-yl]-4H,5H-[1,2,3]triazolo[1,5-a]quinazolin-5-one (Compound 51); or a pharmaceutically acceptable salt thereof.

In some embodiments, the compound of Formula (I) is 3-(2,4-dimethylbenzenesulfonyl)-8-[4-(2-methylpropanoyl)piperazin-1-yl]-4H,5H-[1,2,3]triazolo[1,5-a]quinazolin-5-one (Compound 52); or a pharmaceutically acceptable salt thereof.

In some embodiments, the compound of Formula (I) is 4-[3-(2,4-dimethylbenzenesulfonyl)-5-oxo-4H,5H-[1,2,3]triazolo[1,5-a]quinazolin-8-yl]-1-methylpiperazin-2-one (Compound 53); or a pharmaceutically acceptable salt thereof.

In some embodiments, the compound of Formula (I) is 3-(2,4-dimethylbenzenesulfonyl)-8-{5H,6H,7H,8H-imidazo[1,2-a]pyrazin-7-yl}-4H,5H-[1,2,3]triazolo[1,5-a]quinazolin-5-one (Compound 54); or a pharmaceutically acceptable salt thereof.

In some embodiments, the compound of Formula (I) is 3-(2,4-dimethylbenzenesulfonyl)-8-[4-(oxetan-3-yl)piperazin-1-yl]-4H,5H-[1,2,3]triazolo[1,5-a]quinazolin-5-one (Compound 55); or a pharmaceutically acceptable salt thereof.

In some embodiments, the compound of Formula (I) is 3-(2,4-dimethylbenzenesulfonyl)-8-[4-(oxan-4-yl)piperazin-1-yl]-1H,5H-[1,2,3]triazolo[1,5-a]quinazolin-5-one (Compound 56); or a pharmaceutically acceptable salt thereof.

In some embodiments, the compound of Formula (I) is 8-[4-(2-aminoethyl)piperazin-1-yl]-3-(2,4-dimethylbenzenesulfonyl)-4H,5H-[1,2,3]triazolo[1,5-a]quinazolin-5-one (Compound 57); or a pharmaceutically acceptable salt thereof In some embodiments, the compound of Formula (I) is 8-{4-[3-(diethylamino)propanoyl]piperazin-1-yl}-3-(2,4-dimethylbenzenesulfonyl)-4H,5H-[1,2,3]triazolo[1,5-a]quinazolin-5-one (Compound 58); or a pharmaceutically acceptable salt thereof.

In some embodiments, the compound of Formula (I) is 3-(2,4-dimethylbenzenesulfonyl)-8-[4-(1-methylpiperidin-4-yl)piperazin-1-yl]-1H,5H-[1,2,3]triazolo[1,5-a]quinazolin-5-one (Compound 59); or a pharmaceutically acceptable salt thereof.

In some embodiments, the compound of Formula (I) is 8-(4-cyclopropylpiperazin-1-yl)-3-(2,4-dimethylbenzenesulfonyl)-4H,5H-[1,2,3]triazolo[1,5-a]quinazolin-5-one (Compound 60); or a pharmaceutically acceptable salt thereof.

In some embodiments, the compound of Formula (I) is 3-(2,4-dimethylbenzenesulfonyl)-8-(4-methanesulfonylpiperazin-1-yl)-4H,5H-[1,2,3]triazolo[1,5-a]quinazolin-5-one (Compound 61); or a pharmaceutically acceptable salt thereof.

In some embodiments, the compound of Formula (I) is 4-[3-(2,4-dimethylbenzenesulfonyl)-5-oxo-4H,5H-[1,2,3]triazolo[1,5-a]quinazolin-8-yl]piperazine-1-sulfonamide (Compound 62); or a pharmaceutically acceptable salt thereof.

In some embodiments, the compound of Formula (I) is 2-[3-(2,4-Dimethyl-benzenesulfonyl)-5-oxo-4,5-dihydro-[1,2,3]triazolo[1,5-a]quinazolin-8-yl]-2-aza-spiro[3.3]heptane-6-carboxylic acid (Compound 63); or a pharmaceutically acceptable salt thereof.

In some embodiments, the compound of Formula (I) is 1-[3-(2,4-Dimethyl-benzenesulfonyl)-5-oxo-4,5-dihydro-[1,2,3]triazolo[1,5-a]quinazolin-8-yl]-piperidine-4-carboxylic acid (Compound 64); or a pharmaceutically acceptable salt thereof.

### Anti-Virulence Activity of the Inventive Compounds

The inventive compounds are 3-(phenylsulfonyl)-[1,2,3]triazolo[1,5a]quinazolin-5(4h)-one derivatives comprising an N-bound and specifically defined heterocycle at the 8-position of the [1,2,3]triazolo[1,5a]quinazolin-5(4h)-one core. As shown in Example 3 and Table 2, the inventive compounds were tested in an AgrA reporter assay to evaluate their ability to inhibit the expression of genes under control of the P₃ promoter, which is regulated by AgrA. Specifically, the reporter gene lacZ (which encodes the protein product β-galactosidase) was transfected into S. aureus with a plasmid under control of the P₃ promoter. The inventive compounds were serially diluted in lysogeny broth (LB) with and without human serum (HS) or human serum albumin (HSA), and incubated with S. aureus. Following incubation, the S. aureus cells were lysed and treated with 4-methylumbelliferyl-galactopyranoside (MUG). The β-galactosidase produced by expression of lacZ cleaved the MUG into galactopyranose and 4-methylumbelliferone, which was measured by fluorescence. The fluorescence readout thus serves as a proxy for the ability of the inventive compounds to inhibit genes under the control of the P₃ promoter (e.g., in bacteria, preferably in S. aureus).

The inventive compounds were potent inhibitors of the expression of lacZ and retained activity in the presence of 40 mg/mL HSA with IC₅₀ values as low as 3.7 μg/mL. Thus, the inventive compounds retained activity to inhibit expression of AgrA-regulated genes even when incubated with physiologically relevant concentrations of HSA. In contrast, and as shown in Example 3, even though compounds 45, 46 and 58 of WO 2020/109350 inhibited the expression of lacZ at a concentration of less than 1 μg/mL in the AgrA reporter assay in the absence of HSA or HS, when incubated with S. aureus in the presence of HSA (or HS), the compounds either were inactive even at the highest concentration of compound tested (i.e., 128 μg/mL; Compound 45 of WO 2020/109350), or suffered a profound loss of activity (>200-fold) in the presence of HS (compounds 46 and 58 of WO 2020/109350).

Without wishing to be bound, the concentration of 40 mg/mL HSA was chosen because this is the same concentration of HSA found in human serum.

Without wishing to be bound, the ability of the inventive compounds to retain their activity even in the presence of HSA enables the inventive compounds to be administered systemically (e.g., to a subject such as a mammal, preferably a human; and preferably by mouth and/or parenterally) while still maintaining efficacy. Thus, the inventive compounds were surprisingly able to maintain their activity even when incubated in biologically relevant concentrations of HSA.

### Use of the Inventive Compounds

In a further aspect, the present invention provides a compound according to formula (I) and pharmaceutically acceptable salts, stereoisomers, enantiomers, tautomers of the compounds of formula (I) as disclosed herein for use in a method of reducing the virulence of bacteria, preferably of bacteria expressing AgrA or an ortholog of AgrA, preferably AgrA, and further preferably of bacteria of the genus selected from Staphylococcus, Streptococcus or Clostridium, more preferably of Staphylococcus, and again further preferably of Staphylococcus aureus. In a further very preferred embodiment, said bacteria expresses AgrA or an ortholog of AgrA. In a further very preferred embodiment, said bacteria expresses AgrA. In a further very preferred embodiment, said bacteria is of the genus selected from Staphylococcus, Streptococcus or Clostridium. In a further very preferred embodiment, said bacteria is of the genus Staphylococcus. In a further very preferred embodiment, said bacteria is Staphylococcus aureus. The herein described and disclosed embodiments, preferred embodiments and very preferred embodiments for the compounds of formula (I) should apply to this inventive method irrespective of whether is specifically referred to again or its repetition is avoided for the sake of conciseness.

In a further aspect, the present invention provides a compound according to formula (I) and pharmaceutically acceptable salts, stereoisomers, enantiomers, tautomers of the compounds of formula (I) as disclosed herein for use in a method of preventing or treating a disease, preferably an infection or an inflammatory disease, further preferably a bacterial infection or an inflammatory skin disease, caused or exacerbated by bacteria, wherein preferably said bacteria is selected from the genus of Staphylococcus, Streptococcus or Clostridium, more preferably of Staphylococcus, and again further preferably wherein said bacteria is Staphylococcus aureus. In a further very preferred embodiment, said disease is an infection or an inflammatory disease. In a further very preferred embodiment, said disease is an infection. In a further very preferred embodiment, said disease is a bacterial infection or an inflammatory skin disease, caused or exacerbated by bacteria. In a further very preferred embodiment, said disease a bacterial infection, wherein said bacteria is selected from the genus of Staphylococcus, Streptococcus or Clostridium. In a further very preferred embodiment, said disease is exacerbated by bacteria of the genus selected from Staphylococcus, preferably from Staphylococcus aureus. In a further very preferred embodiment, said disease is an infection caused or exacerbated by bacteria selected from the genus of Staphylococcus. In a further very preferred embodiment, said disease is an infection caused or exacerbated by Staphylococcus aureus. In a further very preferred embodiment, said disease is an inflammatory skin disease, preferably atopic dermatitis or Netherton syndrome. In a further very preferred embodiment, said disease is an inflammatory skin disease, preferably atopic dermatitis exacerbated by Staphylococcus aureus. The herein described and disclosed embodiments, preferred embodiments and very preferred embodiments for the compounds of formula I should apply to this inventive method irrespective of whether is specifically referred to again or its repetition is avoided for the sake of conciseness.

In a further aspect, the present invention provides a compound according to formula (I) for use in a method of inhibition of the quorum sensing, preferably AgrA quorum sensing, in bacteria, preferably in bacteria of the genus selected from Staphylococcus, Streptococcus or Clostridium, more preferably in Staphylococcus, and again further preferably in Staphylococcus aureus. The herein described and disclosed embodiments, preferred embodiments and very preferred embodiments for the compounds of formula I should apply to this inventive method irrespective of whether is specifically referred to again or its repetition is avoided for the sake of conciseness.

In a further preferred embodiment, said bacteria is selected from Streptococcus pyogenes, Clostridium difficile or Staphylococcus aureus, preferably said bacteria is Staphylococcus aureus. In a very preferred embodiment, said bacteria is Staphylococcus aureus.

Thus, the inventive compositions and compounds represent anti-virulence therapeutics which may be used as standalone therapy to strengthen the infected host's self-defense and -healing capacity through reduction of tissue damage, reduction in inflammation, reduction in disease dissemination, full immune response and reduction in reoccurrence rate. In cases where the disarmed pathogen may not be sufficiently cleared by the host, a combination therapy with traditional antibiotics (for infections caused by S. aureus) or with anti-inflammatory agents (for diseases exacerbated by S. aureus) might be considered, allowing eventually to optimize the timeliness and/or the dose of therapy (Dickey S W, et al. (2017) Nat Rev Drug Discov 16(7):457-471).

In a preferred embodiment of the inventive method, said compound of formula (I) inhibits the synthesis of one or more virulence factors by the bacteria, wherein said one or more virulence factor is selected from the group consisting of one or more of toxins (e.g., α, β, γ, γ-variant, and δ-hemolysins, PSMs (e.g., PSMa), Panton-Valentine leukocidin (PVL), leukotoxin E and D (LukED), leukotoxin G and H (LukGH), enterotoxins (e.g., enterotoxin B), exfoliative toxin), proteases (e.g., serine proteases, metalloproteases and cysteine proteases), nuclease, lipase, coagulase, hyaluronidase, clumping factor, pyrogenic toxin superantigen (e.g., TSST-1), and combinations thereof. Thus, in a further preferred embodiment of the inventive method, said one toxin is selected from α, β, γ, γ-variant, and δ-hemolysins. In a further preferred embodiment of the inventive method, said PSM is PSMa. In a further preferred embodiment of the inventive method, said enterotoxin is enterotoxin B or exfoliative toxin. In a further preferred embodiment of the inventive method, said protease is selected from serine proteases, metalloproteases and cysteine proteases. In a further preferred embodiment of the inventive method, said pyrogenic toxin superantigen is TSST-1. In a further preferred embodiment of the inventive method, said compound of formula (I) inhibits the synthesis of one or more virulence factors by the bacteria, wherein said one or more virulence factor is selected from the group consisting of α, β, γ, γ-variant, and δ-hemolysin, PSMa, Panton-Valentine leukocidin (PVL), leukotoxin E and D (LukED), leukotoxin G and H (LukGH), enterotoxin B, exfoliative toxin, a serine protease, a metalloprotease, a cysteine protease, a nuclease, a lipase, a coagulase, a hyaluronidase, a clumping factor, TSST-1, and any combination of one or more of any specific virulence factor or generic group of virulence factors thereof. In a further very preferred embodiment of the inventive method, said compound of formula (I) inhibits the expression of PSMa, RNAIII and/or any of its downstream targets. In a further very preferred embodiment of the inventive method, said compound of formula (I) inhibits the expression of PSMa. In a further very preferred embodiment of the inventive method, said compound of formula (I) inhibits the expression of RNAIII. In a further very preferred embodiment of the inventive method, said compound of formula (I) inhibits the expression of a downstream target of RNAIII. In a further very preferred embodiment, said method further comprises administering an antibiotic or an anti-inflammatory agent to said subject, preferably to said human. In a further very preferred embodiment, said method further comprises administering an antibiotic to said subject, preferably to said human. In a further very preferred embodiment, said method further comprises administering an anti-inflammatory agent, preferably to said human.

In a further aspect, the present invention provides for a method of preventing or treating a disease of a subject in need thereof, preferably an infection or an inflammatory disease, further preferably a bacterial infection or an inflammatory skin disease, caused or exacerbated by bacteria, wherein preferably said bacteria is selected from the genus of Staphylococcus, Streptococcus or Clostridium, more preferably of Staphylococcus, and again further preferably wherein said bacteria is Staphylococcus aureus, said method comprises administering to the subject in need of such prevention or treatment an effective amount of a compound of formula (I), a pharmaceutical composition or a combination product, preferably a compound of formula (I). In a further very preferred embodiment, said disease is an infection or an inflammatory disease. In a further very preferred embodiment, said disease is an infection. In a further very preferred embodiment, said disease is a bacterial infection. In a further very preferred embodiment, said disease is an inflammatory skin disease caused or exacerbated by bacteria. Preferably said infection, preferably said bacterial infection is caused by bacteria of the genus selected from Staphylococcus, Streptococcus or Clostridium, more preferably by Staphylococcus, and again further preferably by Staphylococcus aureus. In a further very preferred embodiment, said aid infection, preferably said bacterial infection comprises an antibiotic resistant Staphylococcus infection, preferably said antibiotic resistant Staphylococcus infection comprises a Methicillin-resistant Staphylococcus aureus infection.

Thus, an inventive compound of formula (I) or an inventive composition, preferably an inventive pharmaceutical composition, comprising an inventive compound of formula (I), as described herein, can be administered to a subject to inhibit the activity of AgrA thereby preventing the production of virulence factors that aid in bacterial infection or development of a disease condition or disorder associated with the bacterial infection. Examples of diseases and disorders associated with a bacterial infection responsive to treatment with the inventive compounds and/or compositions can include, without limitation, skin and soft tissue infections, lung infections, Netherton syndrome or chronic inflammatory skin diseases such as atopic dermatitis.

In a further aspect, the present invention provides a method for inhibiting the quorum sensing, preferably AgrA quorum sensing, in bacteria, preferably in bacteria of the genus selected from Staphylococcus, Streptococcus or Clostridium, more preferably in Staphylococcus, and again further preferably in Staphylococcus aureus, wherein said comprises administering to a subject an effective amount of a compound of formula (I), a pharmaceutical composition or a combination product, preferably a compound of formula (I), in accordance.

In a further preferred embodiment of the present invention, said compound of formula (I) is provided in a topical composition with a pharmaceutically acceptable carrier and is topically administered to a subject, wherein preferably subject has a disease or disorder associated with a bacterial infection, wherein said bacterial infection is an infection caused or exacerbated by bacteria, preferably by Staphylococcus aureus. In a further preferred embodiment said infection caused or exacerbated by bacteria is SSTI, Netherton syndrome or atopic dermatitis. In a further preferred embodiment said bacterial infection is SSTI or atopic dermatitis.

Thus, an inventive compound of formula (I) or an inventive composition or an inventive combination product, preferably an inventive compound of formula (I) inventive pharmaceutical composition, comprising an inventive compound of formula (I), as described herein, can be used to prevent or treat infection of a subject by any bacteria species that utilizes the AgrA response regulator in quorum sensing and the production of virulence factors. The inventive compounds and compositions are typically and preferably administered to subjects having or at risk of having an infection, preferably a bacterial infection such as a Staphylococcus and/or Streptococcus infection. For example, a subject that can benefit from treatment with an inventive compound or composition, as described herein, can be a hospital patient at risk of developing nosocomial infection or a subject known to be infected with or having been exposed to antibiotic resistant bacteria such as, for example, Methicillin-resistant S. aureus, Vancomycin-intermediary-sensible S. aureus, and Vancomycin-resistant S. aureus. Methods of detecting the presence of a Staphylococcus bacterial infection are well known, for example, by culturing from a sample from the subject, e.g. a blood culture, can be used.

In a further aspect, the present invention provides a method for the treatment of a subject suffering from an infection or an inflammatory disease, preferably a bacterial infection or an inflammatory skin disease, caused by bacteria of the genus selected from Staphylococcus, Streptococcus or Clostridium, more preferably by Staphylococcus, and again further preferably by Staphylococcus aureus, said method comprising administering to the subject a compound of formula (I) in accordance with the present invention and at least one antibiotic active against bacteria, preferably against bacteria of the genus selected from Staphylococcus, Streptococcus or Clostridium, more preferably against Staphylococcus, and again further preferably against Staphylococcus aureus.

In a further aspect, the present invention provides a method for preventing or treating a bacterial infection in a subject in need thereof comprising the step of administering an effective amount of a compound of formula (I) in accordance with the present invention to said subject in need of such prevention or treatment. Preferably said bacterial infection is caused by bacteria of the genus selected from Staphylococcus, Streptococcus or Clostridium, more preferably by Staphylococcus, and again further preferably by Staphylococcus aureus.

In a further aspect, the present invention provides a method of preventing or treating a disease caused or exacerbated by Gram-positive quorum sensing bacteria, comprising administering to a subject an effective amount of a compound of formula (I) in accordance with the present invention to a subject in need of such prevention or treatment.

In one aspect, the present disclosure provides a pharmaceutical composition comprising at least one compound according to Formula (I), or a pharmaceutically acceptable salt, tautomer, solvate or hydrate thereof, and a pharmaceutically acceptable excipient.

In one aspect, the present disclosure provides a compound according to Formula (I) or a pharmaceutically acceptable salt, tautomer, solvate or hydrate thereof, or a pharmaceutical composition comprising a compound according to Formula (I), for use as a medicament.

In one aspect, the invention provides an inventive compound as described herein or a pharmaceutical composition comprising an inventive compound as described herein for use in a method of reducing the virulence of a bacteria, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In one aspect, the invention provides an inventive compound as described herein or a pharmaceutical composition comprising an inventive compound as described herein for use in a method of inhibiting the quorum sensing in bacteria, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In one aspect, the invention provides an inventive compound as described herein or a pharmaceutical composition comprising an inventive compound as described herein for use in a method of preventing or treating diseases caused or exacerbated by bacteria, preferably a bacteria of a genus selected from Staphylococcus, Streptococcus or Clostridium, more preferably of Staphylococcus, and again further preferably is Staphylococcus aureus, preferably wherein the disease is a skin disease or a lung disease, more preferably wherein the skin disease is atopic dermatitis, Netherton syndrome or psoriasis.

In some embodiments, the invention provides the use of an inventive compound as described herein or the use of a pharmaceutical composition comprising an inventive compound as described herein in a method of reducing the virulence of a bacteria, preferably in a subject in need thereof, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In some embodiments, the invention provides the use of an inventive compound as described herein or the use of a pharmaceutical composition comprising an inventive compound as described herein in a method of inhibiting the quorum sensing in bacteria, preferably in a subject in need thereof, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In some embodiments, the invention provides the use of an inventive compound as described herein or the use of a pharmaceutical composition comprising an inventive compound as described herein in a method of preventing or treating diseases caused or exacerbated by bacteria, preferably in a subject in need thereof, preferably wherein the bacteria is of a genus selected from Staphylococcus, Streptococcus or Clostridium, more preferably of Staphylococcus, and again further preferably is Staphylococcus aureus, preferably wherein the disease is a skin disease or a lung disease, more preferably wherein the skin disease is atopic dermatitis, Netherton syndrome or psoriasis.

In some embodiments, the invention provides the use of an inventive compound as described herein or the use of a pharmaceutical composition comprising an inventive compound as described herein in the manufacture of a medicament for reducing the virulence of a bacteria, preferably in a subject in need thereof, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In some embodiments, the invention provides the use of an inventive compound as described herein or the use of a pharmaceutical composition comprising an inventive compound as described herein in the manufacture of a medicament for inhibiting the quorum sensing in bacteria, preferably in a subject in need thereof, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In some embodiments, the invention provides the use of an inventive compound as described herein or the use of a pharmaceutical composition comprising an inventive compound as described herein in the manufacture of a medicament for preventing or treating diseases caused or exacerbated by bacteria, preferably in a subject in need thereof, preferably wherein the bacteria is of a genus selected from Staphylococcus, Streptococcus or Clostridium, more preferably of Staphylococcus, and again further preferably is Staphylococcus aureus, preferably wherein the disease is a skin disease or a lung disease, more preferably wherein the skin disease is atopic dermatitis, Netherton syndrome, or psoriasis.

In some embodiments, the invention provides a method of treating a bacterial infection in a subject in need thereof, the method comprising administering to the subject an inventive compound as described herein or a pharmaceutical composition comprising an inventive composition as described herein, preferably wherein the virulence of the bacteria is reduced; preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In some embodiments, the invention provides a method of treating a bacterial infection in a subject in need thereof, the method comprising administering to the subject an inventive compound as described herein or a pharmaceutical composition comprising an inventive composition as described herein, preferably wherein the quorum sensing of the bacteria is inhibited, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA.

In some embodiments, the invention provides a method of treating a disease caused or exacerbated by a bacterial infection in a subject in need thereof, the method comprising administering to the subject an inventive compound as described herein or a pharmaceutical composition comprising an inventive composition as described herein, preferably wherein the bacteria is of a genus selected from Staphylococcus, Streptococcus or Clostridium, more preferably of Staphylococcus, and again further preferably is Staphylococcus aureus, preferably wherein the disease is a skin disease or a lung disease, more preferably wherein the skin disease is atopic dermatitis, Netherton syndrome or psoriasis.

In some embodiments, the invention provides a method of reducing the virulence of a bacteria, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA, comprising exposing the bacteria to an inventive compound as described herein or a pharmaceutical composition comprising an inventive compound as described herein.

In some embodiments, the invention provides a method of inhibiting the quorum sensing in bacteria, preferably wherein the bacteria is of a genus selected from Staphylococcus, Streptococcus or Clostridium, more preferably of Staphylococcus, and again further preferably is Staphylococcus aureus, preferably wherein the bacteria expresses AgrA or an ortholog of AgrA, more preferably wherein the bacteria expresses AgrA, comprising exposing the bacteria to an inventive compound as described herein or a pharmaceutical composition comprising an inventive compound as described herein.

In some embodiments, the invention provides a method of preventing or treating diseases caused or exacerbated by bacteria, preferably wherein the bacteria is of a genus selected from Staphylococcus, Streptococcus or Clostridium, more preferably of Staphylococcus, and again further preferably is Staphylococcus aureus, preferably wherein the disease is a skin disease or a lung disease, more preferably wherein the skin disease is atopic dermatitis, Netherton syndrome or psoriasis, comprising exposing the bacteria to an inventive compound as described herein or a pharmaceutical composition comprising an inventive compound as described herein.

The inventive compounds of formula (I), pharmaceutical compositions comprising the same or combination products can be administered to any subject that can experience the beneficial effects of the inventive compounds, compositions or products, as described herein. Preferably said subject is a human. The inventive compounds, compositions comprising the same or products as described herein can be administered by any means that achieve their intended purpose. For example, administration can be by parenteral, topical, local, subcutaneous, oral, intravenous, intraarticular, intrathecal, intramuscular, intraperitoneal, intradermal, transdermal, buccal, oromucosal, ocular, or via inhalation. Preferably the administration is oral administration or parenteral administration. In preferred embodiments, administration to a subject is systemic (e.g., by oral administration or parenteral administration). In other embodiments, administration to a subject is local such as in a topical solution, topical ointment, or topical cream.

### EXAMPLES

The invention will now be illustrated by way of the following non-limiting examples. While particular embodiments of the invention are described below a skilled person will appreciate that various changes and modifications can be made. References to preparations carried out in a similar manner to, or by the general method of, other preparations, may encompass variations in routine parameters such as time, temperature, workup conditions, minor changes in reagents amounts, and the like.

### Abbreviations

The following list provides definitions of certain abbreviations and symbols as used herein. It will be appreciated that the list is not exhaustive, but the meaning of those abbreviations and symbols not herein below defined will be readily apparent to those skilled in the art. In describing the invention, chemical elements are identified in accordance with the Periodic Table of the Elements.

| | |
|---|---|
| Ar | argon |
| DCM | dichloromethane |
| DMF | dimethylformamide |
| eq. | equivalent |
| EtOAc | ethyl acetate |
| EtOH | ethanol |
| Et₂O | diethyl ether |
| h | hour(s) |
| HPLC | high performance liquid chromatography |
| HS | Human serum |
| HSA | Human serum albumin |
| LC | liquid chromatography |
| M | molar |
| mCPBA | meta-chloroperbenzoic acid |
| MS | mass spectroscopy |
| min | minutes |
| NMR | nuclear magnetic resonance spectroscopy |
| rt | room temperature |
| Rt | Retention time |
| sat. | saturated |
| UPLC | Ultra-high performance liquid chromatography |

Analysis of the compounds: unless specified otherwise, the purity and identity of Intermediate or example compounds were assessed by state-of-the-art H(U)LPC-MS. Methods are described below:

### Methods A: HPLC-MS

HPLC-MS analysis was performed on LC-MS Waters Alliance Micromass ZQ 2000 system equipped with a Waters 2747 sample manager, a Waters 2695 separations module, a Waters 2996 photodiode array detector (200-800 nm) and a Waters Micromass ZQ2000 detector (scan100-800). XBridge C18 column (3.5 μm particle size, dimensions 50×4.6 mm) was used for HPLC analysis. The injection volume was 20 μL. A mixture of water and acetonitrile was used as mobile phase in gradient-elution. The pH of the mobile phase was adjusted with HCOOH and NH₄OH to form a buffer solution at pH 3.8. The analysis time was 5 min (Method A1) or 10 min (Method A2) using a gradient starting from 100% aqueous buffer pH=3.8±0.3 (NH₄OH 0.01%, HCOOH 0.02%) and reaching 100% acetonitrile buffer (NH₄OH 0.01%, HCOOH 0.02%):
(i) within 3.25 min at a flow rate of 2 mL/min (5 min run); or
(ii) within 6.5 min at a flow rate of 2 mL/min (10 min run).

### Methods B: UPLC-MS

UPLC-MS analysis was performed on Waters Acquity UPLC system coupled with SQD mass spectrometer (scan 100-1500) and a photodiode array detector (210-350 nm). BEH C18 column (1.71 μm particle size, dimensions 50×2.1 mm) was used for UPLC analysis. The injection volume was 2 μL. A mixture of water and acetonitrile was used as mobile phase in gradient-elution. The pH of the mobile phase was adjusted with HCOOH and NH₄OH to form a buffer solution at pH 10.

The analysis time was 8 min (Method B1) or 12 min (Method B2) at a flow rate of 0.9 mL/min using a gradient starting from 97% aqueous buffer (10 mM aqueous solution of ammonium formate for solution at pH=10 (high) or 0.1% v/v aqueous solution of formic acid for solution at pH=3 (low)):
for 6.8 min and reaching 97% acetonitrile within 1.1 min (8 min run);
for 1.5 min and reaching 97% acetonitrile within 10 min (12 min run—high pH); or
for 9.5 min and reaching 97% acetonitrile within 0.5 min (12 min run—low pH)—in this case, the flow rate was 0.5 mL/min.

### Intermediate I-1: 8-bromo-3-(2,4-dimethylphenyl)sulfonyl-4H-triazolo[1,5-al]quinazolin-5-one

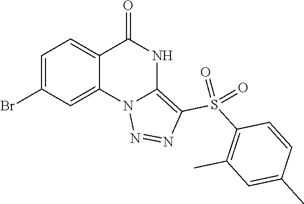 [SMILES: Cc1ccc(S(=O)(=O)c2nnn3c2[nH]c(=O)c2ccc(Br)cc23)c(C)c1]

To an ice-cooled suspension of 2,4-dimethylbenzenethiol (9.8 ml, 72.46 mmol) and potassium carbonate (20 g, 144.7 mmol) in dry DMF (200 ml) was added 2-bromoacetonitrile (76.09 mmol). The reaction mixture was stirred for 1.5 h (on ice). Water (350 ml) was then added, and the product was extracted with Et₂O (3×100 ml). The organic layers were combined, washed with brine (3×80 ml), dried over MgSO₄, filtered and evaporated to afford the intermediate 2-(2,4-dimethylphenyl)-sulfanylacetonitrile as a colourless oil (99.8% yield, >99% purity by HPLC). 2-(2,4-dimethylphenyl)-sulfanylacetonitrile was solubilized in DCM (250 ml) and cooled in an ice bath. mCPBA (<77%, 33 g, 147.2 mmol) was then added portionwise. The reaction mixture was stirred for 40 min on ice and was left at rt overnight. The reaction mixture was then diluted with DCM (250 ml), washed with 5% aq. Na₂S₂O₃ (2×100 ml) and conc. aq. NaHCO₃ (8×200 ml). The combined aqueous were extracted with DCM (2×100 ml). Organic phases were combined and dried over MgSO₄, filtered and evaporated to yield 2-(2,4-dimethylphenyl)-sulfonylacetonitrile as a white powder (92.1% yield, 98% purity by HPLC).

To a cooled (4° C.) solution of methyl 2-amino-4-bromo-benzoate (7 g, 30.4 mmol) in dry acetonitrile, under Ar atmosphere, tert-butyl nitrite (1.5 eq.) was added dropwise. After 20 min stirring, azidotrimethylsilane (1.2 eq.) was added dropwise (exothermic reaction—reaction kept below 15° C.). After addition was completed, the reaction mixture was stirred for 40 min in an ice bath and then allowed to reach rt and stirred for another 2 h. Concentrated aq. NaHCO₃ solution (500 ml) was added to the reaction mixture leading to a suspension which was extracted with EtOAc (3×200 ml). The combined organic layers were washed with brine (200 ml) dried over MgSO₄, filtered and evaporated to afford methyl 2-azido-4-bromo-benzoate as an orange oil (97% yield, 99% purity by HPLC).

To an ice-cooled solution of 2-(2,4-dimethylphenyl)-sulfonylacetonitrile (11.67 g, 54.65 mmol) in dry EtOH (560 ml) was added dropwise a freshly prepared solution of sodium ethoxide in EtOH (15%, 117 mmol). After 30 min, a solution of methyl 2-azido-4-bromo-benzoate (54.13 mmol) in dry EtOH (225 ml) was added dropwise. The resulting mixture was stirred on ice for 1 h and then 1.5 h at rt and then concentrated under vacuum. The pH was adjusted to 3.1 with aq. hydrochloric acid (2M). The precipitate was filtered off. The solid was washed with water (3×400 ml) and dried under vacuum. The crude product was triturated several times with cold EtOAc and Et₂O and dried overnight to yield the title Intermediate I-1 as a beige solid (86.6% yield, 99.1% purity by HPLC).

### Intermediate I-2: 3-(2,4-dimethylphenyl)sulfonyl-8-fluoro-4H-triazolo[1,5-a]quinazolin-5-one

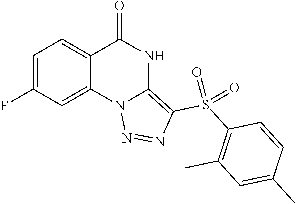 [SMILES: Cc1ccc(S(=O)(=O)c2nnn3c2[nH]c(=O)c2ccc(F)cc23)c(C)c1]

### Intermediate I-2 was Synthesized According to the Methods as Described for I-1 Starting from 2,4-dimethylbenzenethiol and methyl 2-amino-4-fluoro-benzoate

### General Reaction Conditions for Buchwald Coupling

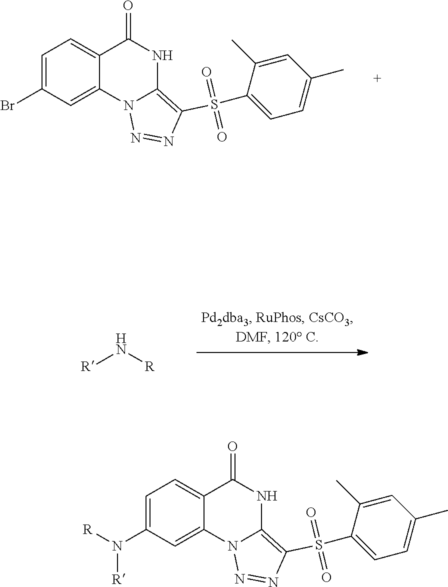

A round-bottom flask was charged with I-1 (1 eq), the corresponding amine (1-3 eq), Pd₂dba₃ (5 mol %), Cs₂CO₃ (1.5 eq) and RuPhos (10 mol %), and DMF (0.035 M) was added. The mixture was degassed by Ar flush then equipped with a reflux condenser and stirred in a preheated heating block at 120° C. until completion (TLC or LC-MS monitoring). After this time the mixture was cool down to rt, the mixture was diluted with MeOH and filtered on celite. The crude product was purified by flash chromatography with the appropriate eluent system, then triturated in diethyl ether to afford the desired compound as solid.

### General Reaction Conditions for Nucleophilic Aromatic Substitution (SNAr) Reaction

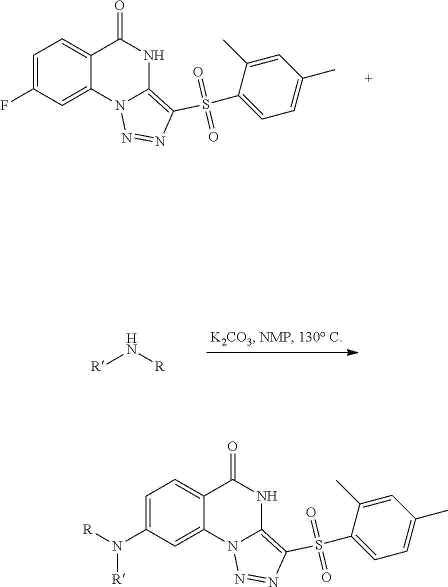

A round-bottom flask was charged with I-1 (1 eq), amine (1-5 eq), K₂CO₃ and NMP (0.1 M). The mixture was stirred at 130° C. until completion (TLC or LC-MS monitoring). Subsequently NMP was removed under vacuum and the crude product was diluted with EtOAc, washed with NH₄Cl (aq. sat.), the organic layer was dried over Na₂SO₄ then concentrated under reduced pressure. When necessary, the product was further purified by flash chromatography with the appropriate eluent system to afford the corresponding compound as solid.

### General Reaction Conditions for Boc Protecting Group Removal

A 50 ml flask was charged with the Boc-protected derivative (1 eq) in dry dioxane (0.35 M), then HCl 4 N in dioxane (15 eq) was added dropwise and the mixture stirred at 80° C. The heating was stopped when the reaction was judged completed by LC-MS then the precipitate was filtered and triturated in Et₂O to afford the desired product.

Alternatively, TFA was used: a 50 ml flask was charged with the Boc-protected derivative (1 eq) in dry DCM (1.7 M), then TFA (15 eq) was added dropwise, and the mixture stirred at 40° C. The heating was stopped when the reaction was judged completed by LC-MS, then the mixture was concentrated under reduced pressure and triturated in Et₂O to afford the desired product.

### General Reaction Conditions for Reductive Amination

Preparation of Compound 25 beginning with Compound 24: To the suspension of Compound 24 (1 eq) in DCM (0.12 M), formaldehyde (aq. 37 wt % solution, 4 eq.) was added dropwise and the resulting suspension was stirred at room temperature overnight (the suspension turned into a clear yellowish solution). NaBH(OAc)₃ (2.5 eq) was then added portion-wise and the mixture stirred until completion (monitoring by TLC or LCMS). Water was added and the mixture was extracted twice with DCM. The combined organic layers were washed with brine then dried over MgSO₄ and concentrated to dryness. If necessary, the product was further purified by flash chromatography with the appropriate eluent system.

### General Reaction Conditions for Amide Formation

A reaction vial was charged with 3-(2,4-dimethylphenyl)sulfonyl-8-piperazin-1-yl-4H-triazolo[1,5-a]quinazolin-5-one hydrochloride, Compound 23 (20 mg, 0.042 mmol, 1 eq.), then acetone (1.5 mL), TEA (12 μL, 0.084 mmol, 2 eq) and 2-methylpropanoyl chloride (26.5 μL, 0.25 mmol, 6 eq.) were added and the mixture stirred at 50° C. overnight. After this time, the solvent was removed under reduced pressure. The crude product was purified by flash chromatography with 100% DCM until DCM-MeOH (9-1) to afford 17 mg of a white solid (Compound 52), 79% Yield.

### General Reaction Conditions for Ester Hydrolysis

In a round-bottom flask, the methyl ester of 2-[3-(2,4-Dimethyl-benzenesulfonyl)-5-oxo-4,5-dihydro-[1,2,3]triazolo[1,5-a]quinazolin-8-yl]-2-aza-spiro[3.3]heptane-6-carboxylic acid, (Compound 63) or the methyl ester of 1-[3-(2,4-Dimethyl-benzenesulfonyl)-5-oxo-4,5-dihydro-[1,2,3]triazolo[1,5-a]quinazolin-8-yl]-piperidine-4-carboxylic acid, (Compound 64) (0.296 mmol) was solubilized with a solution of THF/H₂O (50/50, 10 ml) then LiOH monohydrate was added (74 mg, 1.78 mmol, 6 eq). The mixture was stirred for 2 h at RT. After this time the mixture was diluted with EtOAc and NaOH (1N), the water phase was washed with EtOAc twice to remove organic impurities than it was acidified with HCl 1N at pH 2 and extracted twice with EtOAc. The combined organic layers were washed with NaCl brine then dried over MgSO₄ and concentrated to dryness to afford the respective carboxylic acids as white solid, in about 70% yield.

### Example 1: Preparation of 3-(2,4-dimethylbenzenesulfonyl)-7-hydroxy-8-(4-methylpiperazin-1-yl)-4H,5H-[1.2.3]triazolo[1,5-a]quinazolin-5-one (Compound 1)

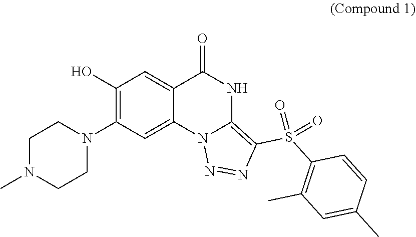

To an ice chilled solution of methyl 4-fluoro-5hydroxy-2-nitro-benzoate (500 mg, 2.3 mmol) and triethylamine (490 μL, 3.516 mmol) in dry DCM (4.6 mL) under Ar atmosphere, was added dropwise a solution of tert-butyldimethylsilyl chloride (530 mg, 3.516 mmol), in dry DCM (3.5 mL). White precipitation occurred during addition. The reaction mixture was stirred on ice for 30 min and then overnight at rt. The mixture was then diluted with DCM (50 ml) and washed with conc. aq. solution of NH₄Cl (3×20 mL). The pooled aqueous phases were back-extracted with DCM (3×20 mL) and the pooled organic phases were concentrated under vacuum. The crude product was purified by flash chromatography (nHex to DCM:MeOH (10:0.5) and after evaporation methyl-5-[tert-butyl(dimethyl)silyl]oxy-4fluoro-2-nitro-benzoate was obtained and used without further purification (62% yield, 84% purity by HPLC).

To a solution of methyl-5-[tert-butyl(dimethyl)silyl]oxy-4fluoro-2-nitro-benzoate (512.5 mg) in MeOH (27 ml), Pd/C (10%, 278 mg, 0.261 mmol) was added and the reaction mixture was stirred under H₂ atmosphere overnight. The mixture was subsequently filtered and the filtrate was evaporated to afford methyl 2-amino-5-[tert-butyl(dimethyl)silyl]oxy-4fluoro-benzoate (463 mg, 99% purity by HPLC).

To an ice chilled solution of methyl 2-amino-5-[tert-butyl(dimethyl)silyl]oxy-4fluoro-benzoate (150 mg, 0.496 mmol) in dry acetonitrile (0.73 ml) under Ar atmosphere, was added dropwise tert-butyl nitrite (90 μL, 0.757 mmol) and 10 min later azidotrimethylsilane (80 μL, 0.603 mmol) (exothermic reaction). The resulting mixture was stirred on ice for another 20 min and then at rt for 2 h. Monitoring by HPLC indicated complete conversion and aq. conc. NaHCO₃ (10 mL) was added. The resulting suspension was extracted with EtOAc (3×15 mL). The combined organic phases were washed with brine (15 mL), dried over Na₂SO₄, filtered and evaporated. Further analysis showed that the silyl protection was removed during the azide formation. Therefore, the crude methyl 2-azido-4-fluoro-5-hydroxy-benzoate (111 mg, 0.499 mmol) was suspended in dry DMF (1.7 mL) and potassium carbonate (138 mg, 0.998 mmol) followed by 1-(chloromethyl)-4-methoxy-benzene (PMB-Cl. 102 μL. 0.752 mmol) were added. The resulting mixture was stirred overnight at rt. Water (40 mL) was then added and the suspension was extracted with EtOAc (3×25 mL). The combined organic layers were washed with brine, dried over Na₂SO₄, filtered and evaporated under vacuum. The crude product was purified by flash chromatography (cy-Hex/EtOAc). After evaporation of the desired fractions, methyl 2-azido-4-fluoro-5-[(4-methoxyphenyl)methoxy]benzoate was isolated as a colourless oil (80% yield, 99% purity).

To a cooled (ice bath) solution of 2-(2,4-dimethyl)sulfonylacetonitrile (84 mg, 0.393 mmol, see I-1 for experimental details) in dry EtOH (0.6 mL) was added dropwise a solution of sodium ethoxide (15%) in EtOH. 15 min after addition, a solution of the previous compound (128.9 mg, 0.385 mmol) in dry EtOH was added dropwise. The resulting mixture was stirred on ice for 1 h and at rt for another 2.5 h. The reaction mixture was then concentrated, and the residue was taken in water (15 mL), then pH was adjusted to 2 with 2M HCl. The precipitate was collected by filtration and washed with water (4×10 mL) and finally dried in a desiccator over KOH. The crude product was purified by reverse-phase chromatography to afford 3-(2,4-dimethylphenyl)sulfonyl-8-fluoro-7-[(4-methoxyphenyl)methoxy]-4H-triazolo[1,5-a]quinazolin-5-one (115 mg, 97% purity) as a brownish solid.

N-methyl-piperazine was introduced following the general procedure for SNAr, affording the PMB-protected title compound as a white solid (117 mg, 96% purity).

The PMB protecting group was removed by stirring the previous intermediate (102 mg) in TFA (1 mL) followed by concentration under nitrogen stream. The residue was diluted in MeOH, neutralized with ammonia and after evaporation, the solid was triturated with several portions of water (20 mL), MeOH (10 mL) and Et₂O (5 mL). After drying, the title compound was obtained as a white solid (117 mg, 98.5% purity, method B1-low).

### Example 2: Preparation of 3-(2,4-dimethylbenzenesulfonyl)-7-hydroxy-8-{6-methyl-2,6-diazaspiro[3.3]heptan-2-yl}-4H,5H-[1,2,3]triazolo[1,5-a]quinazolin-5-one (Compound 2)

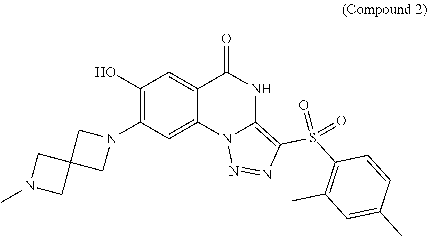

Compound 2 was obtained following the same procedures as described for Compound 1. The title product was isolated as a beige solid (94% purity by HPLC, method B1-low).

Compounds 3 to 59 of the present invention were prepared from the corresponding Intermediate as indicated in the Table 1 below, following the appropriate general protocol(s) described above.

TABLE 1
Exemplary Compounds of the Invention

| | | | Physical data |
|---|---|---|---|
| | | | (Analysis Method)/ |
| | | Intermediate | Rt/MS |
| Cpd | R⁸ substituent | (Protocol(s)) | [M + H+] |
| 3 | 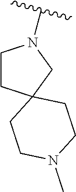 [SMILES: *N1CCC2(CCN(C)CC2)C1] | I-1 (Buchwald) | (A2)/3.73 min/507 |
| 4 | 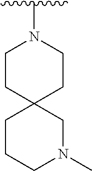 [SMILES: *N1CCC2(CCCN(C)C2)CC1] | I-1 (Buchwald) | (A2)/3.82 min/521 |
| 5 | 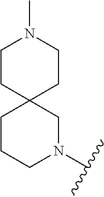 [SMILES: *N1CCCC2(CCN(C)CC2)C1] | I-1 (Buchwald) | (A2)/3.90 min/507 |
| 6 | 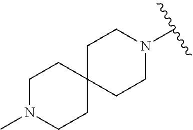 [SMILES: *N1CCC2(CC1)CCN(C)CC2] | I-1 (Buchwald) | (A2)/3.80 min/521 |
| 7 | 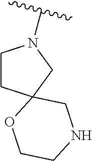 [SMILES: *N1CCC2(CNCCO2)C1] | I-1 (Buchwald, Boc deprotection) | (A2)/3.62 min/495 |
| 8 | 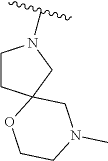 [SMILES: *N1CCC2(C1)CN(C)CCO2] | Ex 7 (reductive amination) | (A2)/3.70 min/509 |
| 9 | 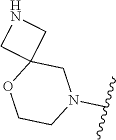 [SMILES: *N1CCOC2(CNC2)C1] | I-1 (Buchwald, Boc deprotection) | (A2)/3.58 min/481 |
| 10 | 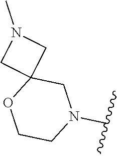 [SMILES: *N1CCOC2(C1)CN(C)C2] | Ex 9 (reductive amination) | (A2)/3.60 min/495 |
| 11 | 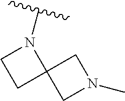 [SMILES: *N1CCC12CN(C)C2] | I-1 (Buchwald) | (A2)/3.57 min/465 |
| 12 | 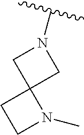 [SMILES: *N1CC2(CCN2C)C1] | I-1 (Buchwald) | (A2)/3.57 min/465 |
| 13 | 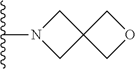 [SMILES: *N1CC2(COC2)C1] | I-1 (Buchwald) | (A2)/4.38 min/452 |
| 14 | 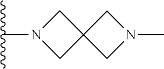 [SMILES: *N1CC2(C1)CN(C)C2] | I-1 (Buchwald) | (A1)/2.23 min/465 |
| 15 | 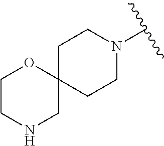 [SMILES: *N1CCC2(CC1)CNCCO2] | I-1 (Buchwald, Boc deprotection) | (A2)/3.75 min/509 |
| 16 | 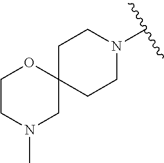 [SMILES: *N1CCC2(CC1)CN(C)CCO2] | I-1 (Buchwald) | (A2)/3.78 min/523 |
| 17 | 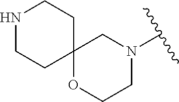 [SMILES: *N1CCOC2(CCNCC2)C1] | I-1 (Buchwald, Boc deprotection) | (A2)/3.68 min/509 |
| 18 | 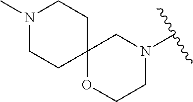 [SMILES: *N1CCOC2(CCN(C)CC2)C1] | I-1 (Buchwald) | (A2)/3.73 min/523 |
| 19 | 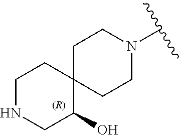 | I-1 (Buchwald, Boc deprotection) | (A2)/3.67 min/523 |
| 20 | 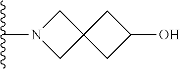 [SMILES: *N1CC2(CC(O)C2)C1] | I-1 (S_{N}Ar) | (B1-low)/4.66 min/466 |
| 21 | 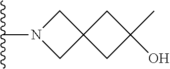 [SMILES: *N1CC2(C1)CC(C)(O)C2] | I-1 (S_{N}Ar) | (B1-low)/4.80 min/480 |
| 22 | 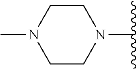 [SMILES: *N1CCN(C)CC1] | I-1 (Buchwald) Or Ex 23 (reductive amination) | (A2/3.48 min/453 |
| 23 | 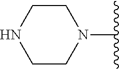 [SMILES: *N1CCNCC1] | I-1 (Buchwald, Boc deprotection) | (A2)/3.48 min/453 |
| 24 | 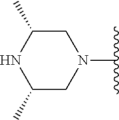 [SMILES: *N1C[C@@H](C)N[C@@H](C)C1] | I-2 (S_{N}Ar, Boc deprotection) | (A1)/2.25 min/467 |
| 25 | 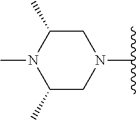 [SMILES: *N1C[C@@H](C)N(C)[C@@H](C)C1] | I-2 (S_{N}Ar) | (B1-low)/3.16 min/481 |
| 26 | 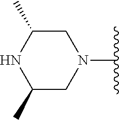 [SMILES: *N1C[C@@H](C)N[C@H](C)C1] | I-2 (S_{N}Ar, Boc deprotection) | (B1-low)/3.18 min/467 |
| | Mixture of trans | | |
| 27 | 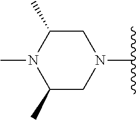 [SMILES: *N1C[C@@H](C)N(C)[C@H](C)C1] | Ex 26 (reductive amination) | (B1-low)/3.19 min/481 |
| | Mixture of trans | | |
| 28 | 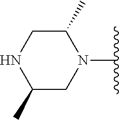 [SMILES: *N1C[C@@H](C)NC[C@@H]1C] | I-1 (Buchwald) | (A2)/3.53 min/467 |
| | Mixture of trans | | |
| 29 | 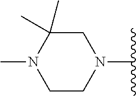 [SMILES: *N1CCN(C)C(C)(C)C1] | I-1 (Buchwald) | (A1)/2.23 min/481 |
| 30 | 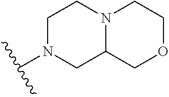 [SMILES: *N1CCN2CCOCC2C1] | I-1 (Buchwald) | (A2)/4.12 min/495 |
| 31 | 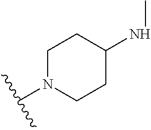 [SMILES: *N1CCC(NC)CC1] | I-1 (Buchwald, Boc deprotection) | (A2)/3.58 min/467 |
| 32 | 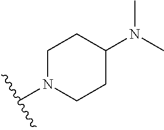 [SMILES: *N1CCC(N(C)C)CC1] | I-1 (Buchwald) | (A2)/3.62 min/481 |
| 33 | 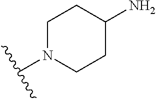 [SMILES: *N1CCC(N)CC1] | I-1 (Buchwald, Boc deprotection) | (B1-low)/3.19 min/453 |
| 34 | 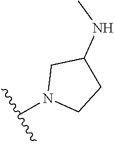 [SMILES: *N1CCC(NC)C1] | I-1 (S_{N}Ar, Boc deprotection) | (B1-low)/3.17 min/453 |
| 35 | 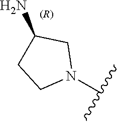 | I-1 (Buchwald, Boc deprotection) | (A2)/3.45 min/439 |
| 36 | 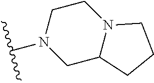 [SMILES: *N1CCN2CCCC2C1] | I-2 (S_{N}Ar) | (B2-low)/4.58 min/479 |
| 37 | 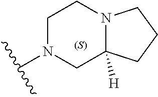 | I-1 (Buchwald) | (A1)/2.22 min/479 |
| 38 | 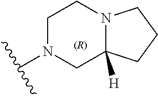 | I-1 (Buchwald) | (A1)/2.22 min/479 |
| 39 | 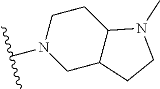 [SMILES: *N1CCC2C(CCN2C)C1] | I-2 (S_{N}Ar) | (B2-low)/4.55 min/493 |
| 40 | 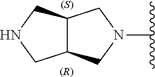 | I-1 (Buchwald, Boc deprotection) | (A2)/3.55 min/465 |
| 41 | 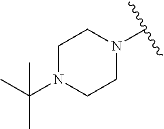 [SMILES: *N1CCN(C(C)(C)C)CC1] | I-1 (Buchwald) | (A1)/2.32 min/495 |
| 42 | 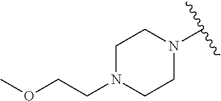 [SMILES: *N1CCN(CCOC)CC1] | I-1 (Buchwald) | (B2-low)/4.69 min/497 |
| 43 | 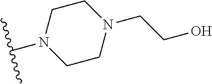 [SMILES: *N1CCN(CCO)CC1] | I-1 (Buchwald) | (A2)/3.45 min/483 |
| 44 | 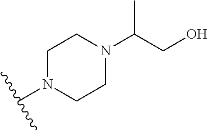 [SMILES: *N1CCN(C(C)CO)CC1] | I-1 (Buchwald) | (A2)/3.48 min/497 |
| 45 | 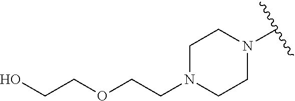 [SMILES: *N1CCN(CCOCCO)CC1] | I-1 (Buchwald) | (B1-low)/2.98 min/527 |
| 46 | 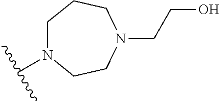 [SMILES: *N1CCCN(CCO)CC1] | I-1 (Buchwald) | (A2)/3.45 min/497 |
| 47 | 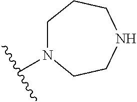 [SMILES: *N1CCCNCC1] | I-1 (Buchwald, Boc deprotection) | (A2)/3.47 min/453 |
| 48 | 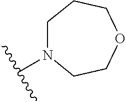 [SMILES: *N1CCCOCC1] | I-1 (Buchwald) | (A2)/4.53 min/454 |
| 49 | 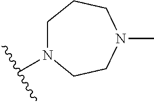 [SMILES: *N1CCCN(C)CC1] | I-1 (Buchwald) | (A2)/3.52 min/467 |
| 50 | 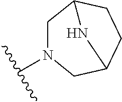 [SMILES: *N1CC2CCC(C1)N2] | I-1 (Buchwald, Boc deprotection) | (B1-low)/3.06 min/465 |
| 51 | 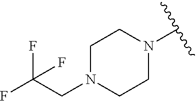 [SMILES: *N1CCN(CC(F)(F)F)CC1] | I-1 (Buchwald) | (B2-low)/7.05 min/521 |
| 52 | 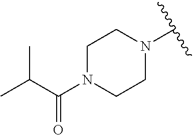 [SMILES: *N1CCN(C(=O)C(C)C)CC1] | Ex 23 (Amide coupling) | (A2)/4.53 min/509 |
| 53 | 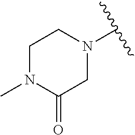 [SMILES: *N1CCN(C)C(=O)C1] | I-1 (Buchwald) | (B1-low)/3.89 min/467 |
| 54 | 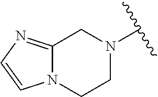 [SMILES: *N1CCn2ccnc2C1] | I-1 (Buchwald) | (B2-low)/4.62 min/476 |
| 55 | 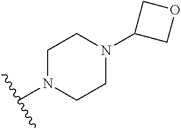 [SMILES: *N1CCN(C2COC2)CC1] | I-1 (Buchwald) | (B1-low)/3.10 min/495 |
| 56 | 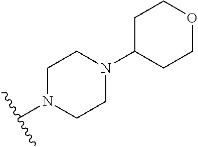 [SMILES: *N1CCN(C2CCOCC2)CC1] | I-2 (S_{N}Ar) | (B1-low)/3.03 min/523 |
| 57 | 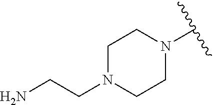 [SMILES: *N1CCN(CCN)CC1] | I-1 (Buchwald, Boc deprotection) | (B1-low)/4.58 min/482 |
| 58 | 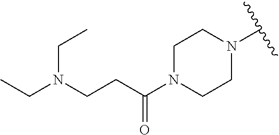 [SMILES: *N1CCN(C(=O)CCN(CC)CC)CC1] | I-2 (SnAr (with Boc- piperazine), Boc deprotection, amide coupling) | (B1-low)/3.35 min/565 |
| 59 | 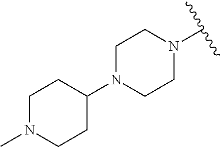 [SMILES: *N1CCN(C2CCN(C)CC2)CC1] | I-1 (Buchwald) | (A2)/3.67 min/536 |
| 60 | 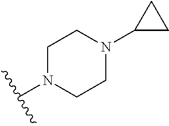 [SMILES: *N1CCN(C2CC2)CC1] | I-1 (Buchwald) | (B2-low)/4.75 min/479 |
| 61 | 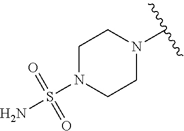 [SMILES: *N1CCN(S(N)(=O)=O)CC1] | I-2 (SnAr) | (A1)/2.20 min/518 |
| 62 | 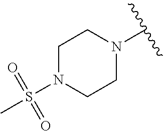 [SMILES: *N1CCN(S(C)(=O)=O)CC1] | I-2 (SNAr) | (A1)/2.70 min/517 |
| 63 | 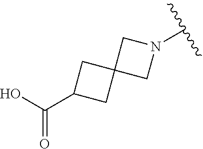 [SMILES: *N1CC2(CC(C(=O)O)C2)C1] | I-1 (Buchwald with the corresponding methyl ester), then ester hydrolysis | (B1-low)/4.06 min/494 |
| 64 | 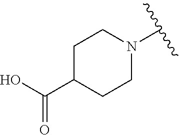 [SMILES: *N1CCC(C(=O)O)CC1] | I-1 (Buchwald with the corresponding methyl ester), then ester hydrolysis | (B1-low)/3.93 min/482 |

### Example 3: AgrA Reporter Assay

The S. aureus USA300 strain UAMS-1625 was transformed with a plasmid expressing the reporter gene lacZ under the control of the P₃ promoter, which is regulated by AgrA. The resulting reporter strain was used to measure AgrA inhibitory activity as follows: From an overnight culture plate, cells were resuspended in 0.9% o (w/v) saline solution and bacterial inoculum was prepared in LB medium with 5×10⁵ CFU/ml. Compounds were two-fold serially diluted in LB and 50 μL of these 10× concentrated samples were added into the 96-deep well plate. 500 μL of bacterial suspension was finally added to the compounds or DMSO control. Plates were covered and incubated with shaking at 37° C. After 18 hours incubation, 100 μL was transferred to a new clear 96-well plate and the OD600 was measured using a TECAN Infinite F200 microplate reader to control for growth inhibition. Another 100 μL was transferred to a new black 96-well plate containing 30 μL of lysis buffer (20 mM Tris/HCl, 3 mM MgCl₂, 0.5% Tween 20 (v/v), 0.5% NP-40 (v/v)) and the plate was incubated with shaking at 37° C. for 60 min. 20 μL of the substrate MUG (4-Methylumbelliferyl j-D-galactopyranoside) at 0.25 mg/ml was added and the fluorescence (excitation: 360 nm, emission: 450 nm) was measured with a TECAN Infinite F200 microplate reader after 3 hours incubation at 37° C. with shaking. The IC₅₀ was determined using GraphPad PRISM after normalization to DMSO control.

IC₅₀ values in the presence of human serum albumin (HSA, 40 mg/mL) were determined by supplementing the LB medium used for inoculum preparation.

The activity of the inventive compounds and Compound 45 of WO 2020/109350 in the absence of human serum albumin and in the presence of 40 mg/mL human serum albumin is given in Table 2, below. Compounds 46 and 58 of WO 2020/109350 were not determined (nd) in 40 mg/mL HSA and their activity ratios were calculated based on their activity in presence of 50% of human serum.

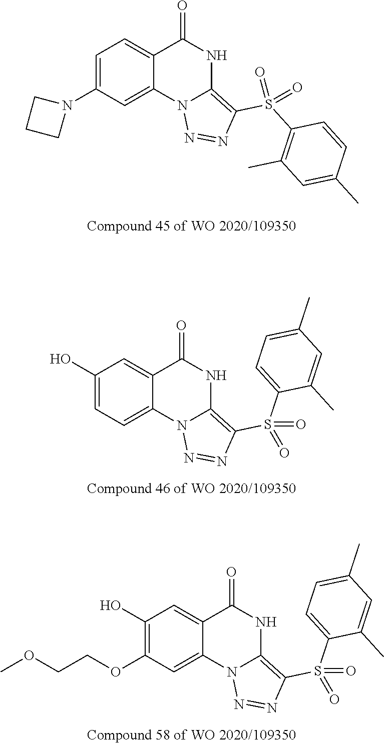

TABLE 2
Reporter Assay Data

| | IC₅₀ | IC₅₀ at | |
|---|---|---|---|
| | without | 40 mg/mL | |
| | HSA | HSA | |
| Compound | (μg/mL) | (μg/mL) | Ratio |
| 45 (WO 2020/109350) | +++ | + | + |
| 46 (WO 2020/109350) | +++ | +* | +* |
| 58 (WO 2020/109350) | +++ | +* | +* |
| 1 | +++ | +++ | +++ |
| 2 | +++ | +++ | +++ |
| 3 | +++ | +++ | +++ |
| 4 | ++ | ++ | +++ |
| 5 | +++ | ++ | +++ |
| 6 | ++ | ++ | +++ |
| 7 | +++ | +++ | +++ |
| 8 | +++ | ++ | +++ |
| 9 | ++ | ++ | +++ |
| 10 | ++ | ++ | +++ |
| 11 | ++ | ++ | +++ |
| 12 | +++ | ++ | +++ |
| 13 | +++ | ++ | ++ |
| 14 | ++ | ++ | +++ |
| 15 | +++ | ++ | +++ |
| 16 | +++ | ++ | ++ |
| 17 | ++ | ++ | +++ |
| 18 | ++ | ++ | +++ |
| 19 | ++ | ++ | +++ |
| 20 | +++ | ++ | ++ |
| 21 | +++ | ++ | ++ |
| 22 | +++ | ++ | +++ |
| 23 | +++ | ++ | +++ |
| 24 | ++ | ++ | +++ |
| 25 | +++ | +++ | +++ |
| 26 | ++ | ++ | +++ |
| 27 | +++ | ++ | +++ |
| 28 | +++ | +++ | +++ |
| 29 | ++ | ++ | ++ |
| 30 | ++ | ++ | +++ |
| 31 | +++ | ++ | +++ |
| 32 | ++ | ++ | +++ |
| 33 | +++ | +++ | +++ |
| 34 | +++ | ++ | ++ |
| 35 | +++ | ++ | +++ |
| 36 | +++ | ++ | +++ |
| 37 | ++ | ++ | +++ |
| 38 | ++ | ++ | +++ |
| 39 | +++ | ++ | +++ |
| 40 | ++ | ++ | +++ |
| 41 | ++ | ++ | +++ |
| 42 | ++ | ++ | +++ |
| 43 | +++ | ++ | ++ |
| 44 | +++ | ++ | ++ |
| 45 | +++ | ++ | +++ |
| 46 | +++ | ++ | +++ |
| 47 | +++ | +++ | +++ |
| 48 | +++ | ++ | ++ |
| 49 | ++ | ++ | +++ |
| 50 | ++ | +++ | +++ |
| 51 | +++ | ++ | ++ |
| 52 | +++ | ++ | +++ |
| 53 | +++ | ++ | ++ |
| 54 | ++ | ++ | ++ |
| 55 | +++ | +++ | ++ |
| 56 | +++ | ++ | +++ |
| 57 | ++ | ++ | +++ |
| 58 | ++ | ++ | +++ |
| 59 | ++ | ++ | +++ |
| 60 | +++ | ++ | ++ |
| 61 | +++ | ++ | ++ |
| 62 | +++ | ++ | ++ |
| 63 | +++ | +++ | +++ |
| 64 | +++ | +++ | +++ |

*Ratio for compounds 46 and 58 of WO 2020/109350 was calculated based on activity in presence of 50% of human serum.
IC₅₀ (without HSA): +++: IC₅₀ ≤ 1 μg/mL, ++: 1 μg/mL < IC₅₀ ≤ 5 μg/mL
IC₅₀ (with HSA): +++: IC₅₀ ≤ 10 μg/mL, ++: 10 μg/mL < IC₅₀ ≤ 50 μg/mL, +: IC₅₀ > 50 μg/mL
Ratio IC₅₀ (in presence of HSA)/IC₅₀ (in absence of HSA): +++ ratio ≤ 25, ++ ratio ≤ 125, +: ratio > 125

1. A compound of Formula (I), or a pharmaceutically acceptable salt, solvate, hydrate, or tautomer thereof: 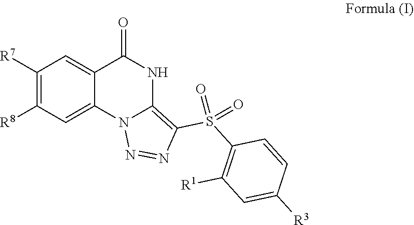
wherein:
R¹ is independently selected from —H, halogen, —C₁-C₆ alkyl, and —C₁-C₆ alkoxy, wherein said alkyl and alkoxy is each optionally substituted with one or more R¹¹;
R³ is independently selected from —H, —C₁-C₆ alkyl, and —C₁-C₆ alkoxy, wherein said alkyl and alkoxy is each optionally substituted by one or more R¹¹;
R⁷ is independently selected from —H and —OH;
R⁸ is a group of the Formula (A): 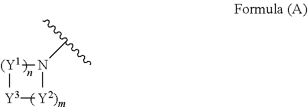
wherein n is independently an integer from 0 to 3;
m is independently an integer from 0 to 3;
Y¹ is independently, at each occurrence, selected from CR⁸¹R⁸², C(O), and O;
Y² is independently, at each occurrence, selected from CR^{81A}R^{82A}, and O;
Y³ is independently selected from CR⁸⁴R⁸⁵; CHR⁸⁶; NR⁸⁷, and O;
wherein the sum of m and n is at least 2 when Y³ is CR⁸⁴R⁸⁵ and wherein the sum of m and n is at least 3 when Y³ is CHR⁸⁶, NR⁸⁷, or O;
R⁸¹, R⁸², R^{81A} and R^{82A} are independently, at each occurrence, selected from —H and —C₁-C₆ alkyl, or one of R⁸¹ and R^{81A}, together with the carbon atoms to which they are attached, can combine to form a bridging ethylene;
R⁸⁴ and R⁸⁵, together with the carbon atom to which they are attached, combine to form a —C₄-C₇ cycloalkyl or a 4- to 7-membered heterocycle, wherein said cycloalkyl and heterocycle is each optionally substituted with one or more halogen, —C₁-C₆ alkyl, —C₁-C₆ haloalkyl, or —OH;
R⁸⁶ is independently selected from —H, —COOH, and —NR⁸⁸R⁸⁹; or R⁸⁶, together with the carbon atom to which it is attached, can combine with one of R⁸¹, R^{81A}, R⁸² or R^{82A} and the carbon atoms to which they are attached to form a 5- to 6-membered heterocycle, wherein said heterocycle is optionally substituted with one or more halogen, —C₁-C₆ alkyl, —C₁-C₆ haloalkyl, —OH, or —COOH;
R⁸⁸ and R⁸⁹ are independently, at each occurrence, selected from —H and —C₁-C₆ alkyl, or one of R⁸⁸ or R⁸⁹, together with the nitrogen atom to which they are attached, can combine with one of R⁸¹, R^{81A}, R⁸² or R^{82A} and the carbon atoms to which they are attached to form a 5- to 6-membered heterocycle;
R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; wherein said heterocycle is optionally substituted with one or more halogen, —C₁-C₆ alkyl, —C₁-C₆ haloalkyl, or —OH;
or R⁸⁷, together with the nitrogen atom to which it is attached, can combine with one of R⁸¹, R^{81A}, R⁸² or R^{82A} and the carbon atoms to which they are attached to form a 5- to 6-membered heterocycle or 5- to 6-membered heteroaryl, wherein said heterocycle or heteroaryl is each optionally substituted with one or more halogen, —C₁-C₆ alkyl, —C₁-C₆ haloalkyl, or —OH; and
R¹¹ is independently, at each occurrence, halogen or —OH.

2. The compound of claim 1, wherein R¹ is —CH₃.

3. The compound of any of the preceding claims, wherein R³ is —CH₃.

4. The compound of any of the preceding claims, wherein R⁷ is —H.

5. The compound of any of the preceding claims, wherein R⁷ is —OH.

6. The compound of any of the preceding claims, wherein Y³ is CR⁸⁴R⁸⁵.

7. The compound of any of claims 1 to 5, wherein Y³ is CHR⁸⁶.

8. The compound of any of claims 1 to 5, wherein Y³ is NR⁸⁷.

9. The compound of claim 8, wherein R⁸⁷ is independently selected from —H, —C₁-C₆ alkyl, —C(O)C₁-C₆ alkyl, —S(O)₂NH₂, —S(O)₂C₁-C₆ alkyl, —C₃-C₆ cycloalkyl, and 4- to 6-membered heterocycle; wherein each alkyl is optionally substituted with one or more halogen, —C₁-C₆ alkoxy, —OH, —O(CH₂)₂—OH, —NH₂, —NHC₁-C₆ alkyl, or N(C₁-C₆ alkyl)₂; wherein said cycloalkyl is optionally substituted with one or more halogen, —C₁-C₆ alkyl, —C₁-C₆ haloalkyl, or —OH; and wherein said heterocycle is optionally substituted with one or more halogen, —C₁-C₆ alkyl, —C₁-C₆haloalkyl, or —OH.

10. The compound of claim 1, wherein R⁸ comprises the Formula (B): 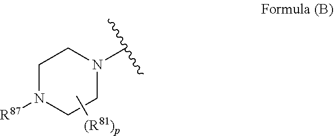
wherein:
R⁸¹ is independently, at each occurrence, selected from —H and —C₁-C₂ alkyl;
R⁸⁷ is independently selected from —H, —C₁-C₂ alkyl, and 4- to 6-membered heterocycle, preferably wherein said heterocycle comprises one heteroatom selected from O and N; and
p is 0, 1 or 2.

11. The compound of claim 1, wherein R⁸ comprises the Formula (C): 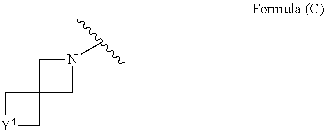
wherein:
Y⁴ is selected from —CR⁹¹R⁹²—, —NR⁹³—, and O;
R⁹¹ and R⁹² are each independently selected from —H, —C₁-C₆ alkyl, and —OH; and
R⁹³ is selected from —H and —C₁-C₆ alkyl.

12. The compound of claim 1, wherein said compound is selected from:
3-(2,4-dimethylbenzenesulfonyl)-7-hydroxy-8-(4-methylpiperazin-1-yl)-4H,5H-[1,2,3]triazolo[1,5-a]quinazolin-5-one (Compound 1);
3-(2,4-dimethylbenzenesulfonyl)-7-hydroxy-8-{6-methyl-2,6-diazaspiro[3.3]heptan-2-yl}-4H,5H-[1,2,3]triazolo[1,5-a]quinazolin-5-one (Compound 2);
3-(2,4-dimethylbenzenesulfonyl)-8-{8-methyl-2,8-diazaspiro[4.5]decan-2-yl}-1H,5H-[1,2,3]triazolo[1,5-a]quinazolin-5-one (Compound 3);
3-(2,4-dimethylbenzenesulfonyl)-8-{2-methyl-2,9-diazaspiro[5.5]undecan-9-yl}-1H,5H-[1,2,3]triazolo[1,5-a]quinazolin-5-one (Compound 4);
3-(2,4-dimethylbenzenesulfonyl)-8-{9-methyl-2,9-diazaspiro[5.5]undecan-2-yl}-1H,5H-[1,2,3]triazolo[1,5-a]quinazolin-5-one (Compound 5);
3-(2,4-dimethylbenzenesulfonyl)-8-{9-methyl-3,9-diazaspiro[5.5]undecan-3-yl}-1H,5H-[1,2,3]triazolo[1,5-a]quinazolin-5-one (Compound 6);
3-(2,4-dimethylbenzenesulfonyl)-8-{6-oxa-2,9-diazaspiro[4.5]decan-2-yl}-4H,5H-[1,2,3]triazolo[1,5-a]quinazolin-5-one (Compound 7);
3-(2,4-dimethylbenzenesulfonyl)-8-{9-methyl-6-oxa-2,9-diazaspiro[4.5]decan-2-yl}-4H,5H-[1,2,3]triazolo[1,5-a]quinazolin-5-one (Compound 8);
3-(2,4-dimethylbenzenesulfonyl)-8-{5-oxa-2,8-diazaspiro[3.5]nonan-8-yl}-4H,5H-[1,2,3]triazolo[1,5-a]quinazolin-5-one (Compound 9);
3-(2,4-dimethylbenzenesulfonyl)-8-{2-methyl-5-oxa-2,8-diazaspiro[3.5]nonan-8-yl}-4H,5H-[1,2,3]triazolo[1,5-a]quinazolin-5-one (Compound 10);
3-(2,4-dimethylbenzenesulfonyl)-8-{6-methyl-1,6-diazaspiro[3.3]heptan-1-yl}-4H,5H-[1,2,3]triazolo[1,5-a]quinazolin-5-one (Compound 11);
3-(2,4-dimethylbenzenesulfonyl)-8-{1-methyl-1,6-diazaspiro[3.3]heptan-6-yl}-4H,5H-[1,2,3]triazolo[1,5-a]quinazolin-5-one (Compound 12);
3-(2,4-dimethylbenzenesulfonyl)-8-{2-oxa-6-azaspiro[3.3]heptan-6-yl}-4H,5H-[1,2,3]triazolo[1,5-a]quinazolin-5-one (Compound 13);
3-(2,4-dimethylbenzenesulfonyl)-8-{6-methyl-2,6-diazaspiro[3.3]heptan-2-yl}-4H,5H-[1,2,3]triazolo[1,5-a]quinazolin-5-one (Compound 14);
3-(2,4-dimethylbenzenesulfonyl)-8-{1-oxa-4,9-diazaspiro[5.5]undecan-9-yl}-1H,5H-[1,2,3]triazolo[1,5-a]quinazolin-5-one (Compound 15);
3-(2,4-dimethylbenzenesulfonyl)-8-{4-methyl-1-oxa-4,9-diazaspiro[5.5]undecan-9-yl}-1H,5H-[1,2,3]triazolo[1,5-a]quinazolin-5-one (Compound 16);
3-(2,4-dimethylbenzenesulfonyl)-8-{1-oxa-4,9-diazaspiro[5.5]undecan-4-yl}-1H,5H-[1,2,3]triazolo[1,5-a]quinazolin-5-one (Compound 17);
3-(2,4-dimethylbenzenesulfonyl)-8-{9-methyl-1-oxa-4,9-diazaspiro[5.5]undecan-4-yl}-1H,5H-[1,2,3]triazolo[1,5-a]quinazolin-5-one (Compound 18);
3-(2,4-dimethylbenzenesulfonyl)-8-[(7R)-7-hydroxy-3,9-diazaspiro[5.5]undecan-3-yl]-1H,5H-[1,2,3]triazolo[1,5-a]quinazolin-5-one (Compound 19);
3-(2,4-dimethylbenzenesulfonyl)-8-{6-hydroxy-2-azaspiro[3.3]heptan-2-yl}-1H,5H-[1,2,3]triazolo[1,5-a]quinazolin-5-one (Compound 20);
3-(2,4-dimethylbenzenesulfonyl)-8-{6-hydroxy-6-methyl-2-azaspiro[3.3]heptan-2-yl}-1H,5H-[1,2,3]triazolo[1,5-a]quinazolin-5-one (Compound 21);
3-(2,4-dimethylbenzenesulfonyl)-8-(4-methylpiperazin-1-yl)-4H,5H-[1,2,3]triazolo[1,5-a]quinazolin-5-one (Compound 22);
3-(2,4-dimethylbenzenesulfonyl)-8-(piperazin-1-yl)-4H,5H-[1,2,3]triazolo[1,5-a]quinazolin-5-one (Compound 23);
3-(2,4-dimethylbenzenesulfonyl)-8-[(3R,5S)-3,5-dimethylpiperazin-1-yl]-4H,5H-[1,2,3]triazolo[1,5-a]quinazolin-5-one (Compound 24);
rac-3-(2,4-dimethylbenzenesulfonyl)-8-[(3R,5S)-3,4,5-trimethylpiperazin-1-yl]-4H,5H-[1,2,3]triazolo[1,5-a]quinazolin-5-one (Compound 25);
3-(2,4-dimethylbenzenesulfonyl)-8-(3,5-dimethylpiperazin-1-yl)-4H,5H-[1,2,3]triazolo[1,5-a]quinazolin-5-one (Compound 26);
3-(2,4-dimethylbenzenesulfonyl)-8-(3,4,5-trimethylpiperazin-1-yl)-4H,5H-[1,2,3]triazolo[1,5-a]quinazolin-5-one (Compound 27);
rac-3-(2,4-dimethylbenzenesulfonyl)-8-[(2R,5S)-2,5-dimethylpiperazin-1-yl]-4H,5H-[1,2,3]triazolo[1,5-a]quinazolin-5-one (Compound 28);
3-(2,4-dimethylbenzenesulfonyl)-8-(3,3,4-trimethylpiperazin-1-yl)-4H,5H-[1,2,3]triazolo[1,5-a]quinazolin-5-one (Compound 29);
3-(2,4-dimethylbenzenesulfonyl)-8-{octahydropyrazino[2,1-c][1,4]oxazin-8-yl}-4H,5H-[1,2,3]triazolo[1,5-a]quinazolin-5-one (Compound 30);
3-(2,4-dimethylbenzenesulfonyl)-8-[4-(methylamino)piperidin-1-yl]-4H,5H-[1,2,3]triazolo[1,5-a]quinazolin-5-one (Compound 31);
8-[4-(dimethylamino)piperidin-1-yl]-3-(2,4-dimethylbenzenesulfonyl)-4H,5H-[1,2,3]triazolo[1,5-a]quinazolin-5-one (Compound 32);
8-(4-aminopiperidin-1-yl)-3-(2,4-dimethylbenzenesulfonyl)-4H,5H-[1,2,3]triazolo[1,5-a]quinazolin-5-one (Compound 33);
3-(2,4-dimethylbenzenesulfonyl)-8-[3-(methylamino)pyrrolidin-1-yl]-4H,5H-[1,2,3]triazolo[1,5-a]quinazolin-5-one (Compound 34);
8-[(3R)-3-aminopyrrolidin-1-yl]-3-(2,4-dimethylbenzenesulfonyl)-4H,5H-[1,2,3]triazolo[1,5-a]quinazolin-5-one (Compound 35);
3-(2,4-dimethylbenzenesulfonyl)-8-{octahydropyrrolo[1,2-a]pyrazin-2-yl}-4H,5H-[1,2,3]triazolo[1,5-a]quinazolin-5-one (Compound 36);
8-[(8aS)-octahydropyrrolo[1,2-a]pyrazin-2-yl]-3-(2,4-dimethylbenzenesulfonyl)-4H,5H-[1,2,3]triazolo[1,5-a]quinazolin-5-one (Compound 37);
8-[(8aR)-octahydropyrrolo[1,2-a]pyrazin-2-yl]-3-(2,4-dimethylbenzenesulfonyl)-4H,5H-[1,2,3]triazolo[1,5-a]quinazolin-5-one (Compound 38);
3-(2,4-dimethylbenzenesulfonyl)-8-{1-methyl-octahydro-1H-pyrrolo[3,2-c]pyridin-5-yl}-4H,5H-[1,2,3]triazolo[1,5-a]quinazolin-5-one (Compound 39);
8-[(3aR,6aS)-octahydropyrrolo[3,4-c]pyrrol-2-yl]-3-(2,4-dimethylbenzenesulfonyl)-4H,5H-[1,2,3]triazolo[1,5-a]quinazolin-5-one (Compound 40);
8-(4-tert-butylpiperazin-1-yl)-3-(2,4-dimethylbenzenesulfonyl)-4H,5H-[1,2,3]triazolo[1,5-a]quinazolin-5-one (Compound 41);
3-(2,4-dimethylbenzenesulfonyl)-8-[4-(2-methoxyethyl)piperazin-1-yl]-4H,5H-[1,2,3]triazolo[1,5-a]quinazolin-5-one (Compound 42);
3-(2,4-dimethylbenzenesulfonyl)-8-[4-(2-hydroxyethyl)piperazin-1-yl]-4H,5H-[1,2,3]triazolo[1,5-a]quinazolin-5-one (Compound 43);
3-(2,4-dimethylbenzenesulfonyl)-8-[4-(1-hydroxypropan-2-yl)piperazin-1-yl]-4H,5H-[1,2,3]triazolo[1,5-a]quinazolin-5-one (Compound 44);
3-(2,4-dimethylbenzenesulfonyl)-8-{4-[2-(2-hydroxyethoxy)ethyl]piperazin-1-yl}-4H,5H-[1,2,3]triazolo[1,5-a]quinazolin-5-one (Compound 45);
3-(2,4-dimethylbenzenesulfonyl)-8-[4-(2-hydroxyethyl)-1,4-diazepan-1-yl]-4H,5H-[1,2,3]triazolo[1,5-a]quinazolin-5-one (Compound 46);
8-(1,4-diazepan-1-yl)-3-(2,4-dimethylbenzenesulfonyl)-4H,5H-[1,2,3]triazolo[1,5-a]quinazolin-5-one (Compound 47);
3-(2,4-dimethylbenzenesulfonyl)-8-(1,4-oxazepan-4-yl)-4H,5H-[1,2,3]triazolo[1,5-a]quinazolin-5-one (Compound 48);
3-(2,4-dimethylbenzenesulfonyl)-8-(4-methyl-1,4-diazepan-1-yl)-4H,5H-[1,2,3]triazolo[1,5-a]quinazolin-5-one (Compound 49);
8-{[(1R,3R,5S)-8-azabicyclo[3.2.1]octan-3-yl]amino}-3-(2,4-dimethylbenzenesulfonyl)-1H,5H-[1,2,3]triazolo[1,5-a]quinazolin-5-one (Compound 50);
3-(2,4-dimethylbenzenesulfonyl)-8-[4-(2,2,2-trifluoroethyl)piperazin-1-yl]-4H,5H-[1,2,3]triazolo[1,5-a]quinazolin-5-one (Compound 51);
3-(2,4-dimethylbenzenesulfonyl)-8-[4-(2-methylpropanoyl)piperazin-1-yl]-4H,5H-[1,2,3]triazolo[1,5-a]quinazolin-5-one (Compound 52);
4-[3-(2,4-dimethylbenzenesulfonyl)-5-oxo-4H,5H-[1,2,3]triazolo[1,5-a]quinazolin-8-yl]-1-methylpiperazin-2-one (Compound 53);
3-(2,4-dimethylbenzenesulfonyl)-8-{5H,6H,7H,8H-imidazo[1,2-a]pyrazin-7-yl}-4H,5H-[1,2,3]triazolo[1,5-a]quinazolin-5-one (Compound 54);
3-(2,4-dimethylbenzenesulfonyl)-8-[4-(oxetan-3-yl)piperazin-1-yl]-4H,5H-[1,2,3]triazolo[1,5-a]quinazolin-5-one (Compound 55);
3-(2,4-dimethylbenzenesulfonyl)-8-[4-(oxan-4-yl)piperazin-1-yl]-1H,5H-[1,2,3]triazolo[1,5-a]quinazolin-5-one (Compound 56);
8-[4-(2-aminoethyl)piperazin-1-yl]-3-(2,4-dimethylbenzenesulfonyl)-4H,5H-[1,2,3]triazolo[1,5-a]quinazolin-5-one (Compound 57);
8-{4-[3-(diethylamino)propanoyl]piperazin-1-yl}-3-(2,4-dimethylbenzenesulfonyl)-4H,5H-[1,2,3]triazolo[1,5-a]quinazolin-5-one (Compound 58);
3-(2,4-dimethylbenzenesulfonyl)-8-[4-(1-methylpiperidin-4-yl)piperazin-1-yl]-1H,5H-[1,2,3]triazolo[1,5-a]quinazolin-5-one (Compound 59);
8-(4-cyclopropylpiperazin-1-yl)-3-(2,4-dimethylbenzenesulfonyl)-4H,5H-[1,2,3]triazolo[1,5-a]quinazolin-5-one (Compound 60);
3-(2,4-dimethylbenzenesulfonyl)-8-(4-methanesulfonylpiperazin-1-yl)-4H,5H-[1,2,3]triazolo[1,5-a]quinazolin-5-one (Compound 61);
4-[3-(2,4-dimethylbenzenesulfonyl)-5-oxo-4H,5H-[1,2,3]triazolo[1,5-a]quinazolin-8-yl]piperazine-1-sulfonamide (Compound 62); and
2-[3-(2,4-Dimethyl-benzenesulfonyl)-5-oxo-4,5-dihydro-[1,2,3]triazolo[1,5-a]quinazolin-8-yl]-2-aza-spiro[3.3]heptane-6-carboxylic acid (Compound 63); and
1-[3-(2,4-Dimethyl-benzenesulfonyl)-5-oxo-4,5-dihydro-[1,2,3]triazolo[1,5-a]quinazolin-8-yl]-piperidine-4-carboxylic acid (Compound 64); or a pharmaceutically acceptable salt thereof.

13. The compound of claim 1, wherein said compound is selected from:
3-(2,4-dimethylbenzenesulfonyl)-7-hydroxy-8-(4-methylpiperazin-1-yl)-4H,5H-[1,2,3]triazolo[1,5-a]quinazolin-5-one (Compound 1);
3-(2,4-dimethylbenzenesulfonyl)-7-hydroxy-8-{6-methyl-2,6-diazaspiro[3.3]heptan-2-yl}-4H,5H-[1,2,3]triazolo[1,5-a]quinazolin-5-one (Compound 2);
3-(2,4-dimethylbenzenesulfonyl)-8-{8-methyl-2,8-diazaspiro[4.5]decan-2-yl}-1H,5H-[1,2,3]triazolo[1,5-a]quinazolin-5-one (Compound 3);
3-(2,4-dimethylbenzenesulfonyl)-8-{6-oxa-2,9-diazaspiro[4.5]decan-2-yl}-4H,5H-[1,2,3]triazolo[1,5-a]quinazolin-5-one (Compound 7);
3-(2,4-dimethylbenzenesulfonyl)-8-{9-methyl-6-oxa-2,9-diazaspiro[4.5]decan-2-yl}-4H,5H-[1,2,3]triazolo[1,5-a]quinazolin-5-one (Compound 8);
3-(2,4-dimethylbenzenesulfonyl)-8-{1-methyl-,6-diazaspiro[3.3]heptan-6-yl}-4H,5H-[1,2,3]triazolo[1,5-a]quinazolin-5-one (Compound 12);
3-(2,4-dimethylbenzenesulfonyl)-8-{6-methyl-2,6-diazaspiro[3.3]heptan-2-yl}-4H,5H-[1,2,3]triazolo[1,5-a]quinazolin-5-one (Compound 14);
3-(2,4-dimethylbenzenesulfonyl)-8-{4-methyl-1-oxa-4,9-diazaspiro[5.5]undecan-9-yl}-1H,5H-[1,2,3]triazolo[1,5-a]quinazolin-5-one (Compound 16);
3-(2,4-dimethylbenzenesulfonyl)-8-{6-hydroxy-6-methyl-2-azaspiro[3.3]heptan-2-yl}-1H,5H-[1,2,3]triazolo[1,5-a]quinazolin-5-one (Compound 21);
3-(2,4-dimethylbenzenesulfonyl)-8-(4-methylpiperazin-1-yl)-4H,5H-[1,2,3]triazolo[1,5-a]quinazolin-5-one (Compound 22);
3-(2,4-dimethylbenzenesulfonyl)-8-(piperazin-1-yl)-4H,5H-[1,2,3]triazolo[1,5-a]quinazolin-5-one (Compound 23);
3-(2,4-dimethylbenzenesulfonyl)-8-[(3R,5S)-3,5-dimethylpiperazin-1-yl]-4H,5H-[1,2,3]triazolo[1,5-a]quinazolin-5-one (Compound 24);
rac-3-(2,4-dimethylbenzenesulfonyl)-8-[(3R,5S)-3,4,5-trimethylpiperazin-1-yl]-4H,5H-[1,2,3]triazolo[1,5-a]quinazolin-5-one (Compound 25);
rac-3-(2,4-dimethylbenzenesulfonyl)-8-[(2R,5S)-2,5-dimethylpiperazin-1-yl]-4H,5H-[1,2,3]triazolo[1,5-a]quinazolin-5-one (Compound 28);
3-(2,4-dimethylbenzenesulfonyl)-8-[4-(methylamino)piperidin-1-yl]-4H,5H-[1,2,3]triazolo[1,5-a]quinazolin-5-one (Compound 31);
3-(2,4-dimethylbenzenesulfonyl)-8-{octahydropyrrolo[1,2-a]pyrazin-2-yl}-4H,5H-[1,2,3]triazolo[1,5-a]quinazolin-5-one (Compound 36);
3-(2,4-dimethylbenzenesulfonyl)-8-{1-methyl-octahydro-1H-pyrrolo[3,2-c]pyridin-5-yl}-4H,5H-[1,2,3]triazolo[1,5-a]quinazolin-5-one (Compound 39);
3-(2,4-dimethylbenzenesulfonyl)-8-{4-[2-(2-hydroxyethoxy)ethyl]piperazin-1-yl}-4H,5H-[1,2,3]triazolo[1,5-a]quinazolin-5-one (Compound 45);
3-(2,4-dimethylbenzenesulfonyl)-8-[4-(2-hydroxyethyl)-1,4-diazepan-1-yl]-4H,5H-[1,2,3]triazolo[1,5-a]quinazolin-5-one (Compound 46);
8-(1,4-diazepan-1-yl)-3-(2,4-dimethylbenzenesulfonyl)-4H,5H-[1,2,3]triazolo[1,5-a]quinazolin-5-one (Compound 47);
3-(2,4-dimethylbenzenesulfonyl)-8-(1,4-oxazepan-4-yl)-4H,5H-[1,2,3]triazolo[1,5-a]quinazolin-5-one (Compound 48);
3-(2,4-dimethylbenzenesulfonyl)-8-[4-(2-methylpropanoyl)piperazin-1-yl]-4H,5H-[1,2,3]triazolo[1,5-a]quinazolin-5-one (Compound 52);
4-[3-(2,4-dimethylbenzenesulfonyl)-5-oxo-4H,5H-[1,2,3]triazolo[1,5-a]quinazolin-8-yl]-1-methylpiperazin-2-one (Compound 53);
3-(2,4-dimethylbenzenesulfonyl)-8-[4-(oxetan-3-yl)piperazin-1-yl]-4H,5H-[1,2,3]triazolo[1,5-a]quinazolin-5-one (Compound 55);
3-(2,4-dimethylbenzenesulfonyl)-8-[4-(oxan-4-yl)piperazin-1-yl]-1H,5H-[1,2,3]triazolo[1,5-a]quinazolin-5-one (Compound 56);
8-(4-cyclopropylpiperazin-1-yl)-3-(2,4-dimethylbenzenesulfonyl)-4H,5H-[1,2,3]triazolo[1,5-a]quinazolin-5-one (Compound 60);
3-(2,4-dimethylbenzenesulfonyl)-8-(4-methanesulfonylpiperazin-1-yl)-4H,5H-[1,2,3]triazolo[1,5-a]quinazolin-5-one (Compound 61);
4-[3-(2,4-dimethylbenzenesulfonyl)-5-oxo-4H,5H-[1,2,3]triazolo[1,5-a]quinazolin-8-yl]piperazine-1-sulfonamide (Compound 62);
2-[3-(2,4-Dimethyl-benzenesulfonyl)-5-oxo-4,5-dihydro-[1,2,3]triazolo[1,5-a]quinazolin-8-yl]-2-aza-spiro[3.3]heptane-6-carboxylic acid (Compound 63); and
1-[3-(2,4-Dimethyl-benzenesulfonyl)-5-oxo-4,5-dihydro-[1,2,3]triazolo[1,5-a]quinazolin-8-yl]-piperidine-4-carboxylic acid (Compound 64); or a pharmaceutically acceptable salt thereof.

14. A pharmaceutical composition comprising at least one compound according to any of the preceding claims, or a pharmaceutically acceptable salt, tautomer, solvate or hydrate thereof, and a pharmaceutically acceptable excipient.

15. A compound according to any of claims 1 to 13 or a pharmaceutically acceptable salt, tautomer, solvate or hydrate thereof, or a pharmaceutical composition according to claim 14, for use as a medicament.
